(19) United States
(12) Patent Application Publication
(10) Pub. No.: US 2023/0157600 A1
(43) Pub. Date: May 25, 2023

(54) INFORMATION PROCESSOR AND INFORMATION PROCESSING PROGRAM

(71) Applicant: SONY GROUP CORPORATION, Tokyo (JP)

(72) Inventors: Naoya SAZUKA, Tokyo (JP); Koki KATSUMATA, Tokyo (JP)

(73) Assignee: SONY GROUP CORPORATION, Tokyo (JP)

(21) Appl. No.: 17/916,978

(22) PCT Filed: Apr. 14, 2021
(86) PCT No.: PCT/JP2021/015440

§ 371 Date: Oct. 4, 2022

(30) Foreign Application Priority Data
Apr. 14, 2020 (JP) 2020-072585
Dec. 7, 2020 (JP) 2020-203058

(51) Int. Cl. A61B 5/16; G16H 50/20

(52) U.S. Cl. CPC A61B 5/162; G16H 50/20

(57) ABSTRACT

An information processor according to an aspect of the present disclosure includes a deriving unit that derives a cognitive capacity of a user on a basis of dispersion of reaction times of the user for a plurality of requests.

### TECHNICAL FIELD

The present disclosure relates to an information processor and an information processing program.

### BACKGROUND ART

It is difficult to know an objective cognitive capacity (cognitive resource) in a simple way when a person is addressing a certain task. Therefore, a judgment of a person regarding a state has been heretofore subjectively determined for both the person and others, or determined by a method that does not evaluate propriety of a physiological state. One reason for this is that, although a person varies his or her action in response to an environmental load, the relationship between the environmental load and a brain function is not well understood.

### CITATION LIST

### Patent Literature

PTL 1: Japanese Unexamined Patent Application Publication No. 2019-000457

### SUMMARY OF THE INVENTION

Incidentally, for example, in an invention described in PTL 1, in a case where answers are successively necessary, tasks are determined by evaluating a gap between actual reaction time and correct reaction time only when there is correct reaction time for an answer. Therefore, it is difficult, in the invention described in PTL 1, to determine a task, in a case where there is no correct reaction time. It is therefore desirable to provide an information processor and an information processing program that make it possible to determine a task, regardless of whether or not there is reaction time or whether or not there is correct reaction time.

An information processor according to a first aspect of the present disclosure includes a deriving unit that derives a cognitive capacity (cognitive resource) of a user on a basis of dispersion of reaction times of the user for a plurality of requests.

In the information processor according to the first aspect of the present disclosure, the cognitive capacity of the user is derived on the basis of the dispersion of the reaction times of the user corresponding to the plurality of requests. This makes it possible to determine a task for the user using the derived cognitive capacity. Here, the present discloser has experimentally obtained knowledge that the dispersion of the reaction times varies depending on tasks. It is therefore possible to determine a task for the user on the basis of the cognitive capacity derived from the dispersion of the reaction times.

An information processor according to a second aspect of the present disclosure includes a deriving unit that derives a cognitive capacity of a user on a basis of a biological signal of the user for a request.

In the information processor according to the second aspect of the present disclosure, the cognitive capacity of the user is derived on the basis of the biological signal of the user for the request. This makes it possible to determine a task for the user using the derived cognitive capacity. Here, the present discloser has experimentally obtained knowledge that the biological signal of the user varies depending on tasks. It is therefore possible to determine a task for the user on the basis of the cognitive capacity derived from the biological signal of the user.

An information processor according to a third aspect of the present disclosure includes a characteristic value generation unit, an evaluation value generation unit, and a deriving unit. The characteristic value generation unit generates a characteristic value of a waveform to be observed for each of pieces of observation data, on a basis of a plurality of pieces of partial observation data in an observation period shorter than a predetermined observation period of each of the pieces of observation data obtained by biological observation of a user in the predetermined period, with the plurality of pieces of partial observation data being included in each of the pieces of observation data. The evaluation value generation unit generates an evaluation value for a difference between the pieces of observation data regarding the waveform to be observed on a basis of the characteristic value generated by the characteristic value generation unit for each of the pieces of observation data. The deriving unit derives a cognitive capacity of the user on a basis of the evaluation value generated by the evaluation value generation unit.

In the information processor according to the third aspect of the present disclosure, the characteristic value for each of the pieces of observation data is derived from each of the pieces of observation data obtained by the biological observation of the user in a predetermined period, and the evaluation value for a difference between the pieces of observation data regarding the waveform to be observed is generated on the basis of the derived characteristic value for each of the pieces of observation data. Then, the cognitive capacity of the user is derived on the basis of the generated evaluation value. Here, the present discloser has experimentally obtained knowledge that the above-described evaluation value varies depending on tasks. It is therefore possible to determine a task for the user on the basis of the cognitive capacity derived from the above-described evaluation value.

An information processing program according to a fourth aspect of the present disclosure causes a computer to derive a cognitive capacity of a user on a basis of dispersion of reaction times of the user for a plurality of requests.

In the information processing program according to the fourth aspect of the present disclosure, the cognitive capacity of the user is derived on the basis of the dispersion of the reaction times of the user for the plurality of requests. Here, the present discloser has experimentally obtained knowledge that the dispersion of the reaction times varies depending on tasks. It is therefore possible to determine a task for the user on the basis of the cognitive capacity derived from the dispersion of the reaction times.

An information processing program according to a fifth aspect of the present disclosure causes a computer to derive a cognitive capacity of a user on a basis of a fluctuation in a biological signal in a specific frequency band of the user for a request.

In the information processor program according to the fifth aspect of the present disclosure, the cognitive capacity of the user is derived on the basis of the fluctuation in the biological signal in the specific frequency band of the user for the request. Here, the present discloser has experimentally obtained knowledge that the fluctuation in the biological signal in the specific frequency band of the user varies depending on tasks. It is therefore possible to determine a task for the user on the basis of the cognitive capacity derived from the fluctuation in the biological signal in the specific frequency band of the user.

An information processing program according to a sixth aspect of the present disclosure causes a computer to:

(1) generate a characteristic value of a waveform to be observed for each of pieces of observation data, on a basis of a plurality of pieces of partial observation data in an observation period shorter than a predetermined observation period of each of the pieces of observation data obtained by biological observation of a user in the predetermined period, the plurality of pieces of partial observation data being included in each of the pieces of observation data;
(2) generate an evaluation value for a difference between the pieces of observation data regarding the waveform to be observed on a basis of the generated characteristic value for each of the pieces of observation data; and
(3) derive a cognitive capacity of the user on a basis of the generated evaluation value.

In the information processing program according to the sixth aspect of the present disclosure, a characteristic value for each of the pieces of observation data is derived from each of the pieces of observation data obtained by the biological observation of the user in a predetermined period, and the evaluation value for a difference between the pieces of observation data regarding the waveform to be observed is generated on the basis of the derived characteristic value for each of the pieces of observation data. Then, the cognitive capacity of the user is derived on the basis of the generated evaluation value. Here, the present discloser has experimentally obtained knowledge that the above-described evaluation value varies depending on tasks. It is therefore possible to derive the cognitive capacity of the user on the basis of the above-described evaluation value, and to determine a task for the user on the basis of the derived cognitive capacity.

An information processor according to a seventh aspect of the present disclosure includes a changing unit that changes a task for a user on a basis of dispersion of reaction times of the user for a plurality of requests.

In the information processor according to the seventh aspect of the present disclosure, the task for the user is changed on the basis of the dispersion of the reaction times of the user corresponding to the plurality of requests. Here, the present discloser has experimentally obtained knowledge that the dispersion of the reaction times varies depending on tasks. It is therefore possible to change the task for the user on the basis of the dispersion of the reaction times.

An information processor according to an eighth aspect of the present disclosure includes a changing unit that changes a task for a user on a basis of a fluctuation in a biological signal of the user.

In the information processor according to the eighth aspect of the present disclosure, the task for the user is changed on the basis of the fluctuation in the biological signal of the user for a request. Here, the present discloser has experimentally obtained knowledge that the biological signal of the user varies depending on tasks. It is therefore possible to change the task for the user on the basis of the fluctuation in the biological signal of the user.

### BRIEF DESCRIPTION OF THE DRAWINGS

FIG. 1 is a diagram illustrating an example of answering time (reaction time) for a large number of successive questions of a low difficulty level.

FIG. 2 is a diagram illustrating an example of answering time (reaction time) for a large number of successive questions of a high difficulty level.

FIG. 3 is a diagram illustrating an example of a power spectrum density of a waveform in FIG. 1.

FIG. 4 is a diagram illustrating an example of a power spectrum density of a waveform in FIG. 2.

FIG. 5 is a diagram illustrating an example of a relationship between a task difference in dispersion of reaction times and a task difference in a peak value of power of a brain wave in a low-frequency band.

FIG. 6 is a diagram illustrating an example of a relationship between a task difference in dispersion of reaction times and a task difference in an accuracy rate.

FIG. 7 is a diagram illustrating an example of a relationship between the task difference in the peak value of the power of the brain wave in the low-frequency band and the task difference in the accuracy rate.

FIG. 8 is a diagram illustrating an example of a relationship between a task difference in an arousal level and a task difference in the peak value of the power of the brain wave in the low-frequency band.

FIG. 9 is a diagram illustrating an example of a relationship between the task difference in the arousal level and the task difference in the accuracy rate.

FIG. 10 is a diagram illustrating an example of a relationship between the dispersion of the reaction times and a task difference in the peak value of the power of the brain wave in the low-frequency band.

FIG. 11 is a diagram illustrating an example of a relationship between the dispersion of the reaction times and the accuracy rate.

FIG. 12 is a diagram illustrating an example of a relationship between the arousal level and the accuracy rate.

FIG. 13 is a diagram illustrating an example of a relationship between the task difference in the dispersion of the reaction times and the accuracy rate.

FIG. 14 is a diagram illustrating an example of a schematic configuration of an information processor according to a first embodiment of the present disclosure.

FIG. 15 is a flowchart illustrating an example of a procedure to change a difficulty level in the information processor in FIG. 14.

FIG. 16 is a flowchart illustrating an example of a procedure to derive a regression formula in the information processor in FIG. 14.

FIG. 17 is a diagram illustrating a modification example of the schematic configuration of the information processor in FIG. 14.

FIG. 18 is a conceptual diagram illustrating an example of a regression table in FIG. 14.

FIG. 19 is a diagram illustrating a modification example of the schematic configuration of the information processor in FIG. 14.

FIG. 20 is a flowchart illustrating an example of a procedure to change a difficulty level in the information processor in FIG. 19.

FIG. 21 is a flowchart illustrating an example of a procedure to derive a regression formula in the information processor in FIG. 19.

FIG. 22 is a diagram illustrating a modification example of the schematic configuration of the information processor in FIG. 14.

FIG. 23 is a flowchart illustrating an example of a procedure to change a difficulty level in the information processor in FIG. 22.

FIG. 24 is a flowchart illustrating an example of a procedure to derive a regression formula in the information processor in FIG. 22.

FIG. 25 is a diagram illustrating an example of a schematic configuration of an information processor according to a second embodiment of the present disclosure.

FIG. 26 is a flowchart illustrating an example of a procedure to change a difficulty level in the information processor in FIG. 25.

FIG. 27 is a flowchart illustrating an example of a procedure to derive a regression formula in the information processor in FIG. 25.

FIG. 28 is a diagram illustrating an example of a schematic configuration of an information processor according to a third embodiment of the present disclosure.

FIG. 29 is a flowchart illustrating an example of a procedure to change a difficulty level in the information processor in FIG. 28.

FIG. 30 is a diagram illustrating an example of a schematic configuration of an information processor according to a fourth embodiment of the present disclosure.

FIG. 31 is a schematic diagram illustrating an example of a learning procedure of a learning model in the information processor in FIG. 30.

FIG. 32 is a schematic diagram illustrating an example of a state estimation procedure by the learning model in the information processor in FIG. 30.

FIG. 33 is a flowchart illustrating an example of a procedure to change a difficulty level in the information processor in FIG. 30.

FIG. 34 is a flowchart illustrating an example of a procedure to derive a regression formula in the information processor in FIG. 30.

FIG. 35 is a diagram illustrating a modification example of the schematic configuration of the information processor in FIG. 30.

FIG. 36 is a flowchart illustrating an example of a procedure to change a difficulty level in the information processor in FIG. 30.

FIG. 37 is a flowchart illustrating an example of a procedure to derive a regression formula in the information processor in FIG. 30.

FIG. 38 is a diagram illustrating a modification example of the schematic configuration of the information processor in FIG. 14.

FIG. 39 is a flowchart illustrating an example of a procedure to change a difficulty level in the information processor in FIG. 38.

FIG. 40 is a flowchart illustrating an example of a procedure to derive a regression formula in the information processor in FIG. 38.

FIG. 41 is a diagram illustrating an example of a head-mounted display mounted with a sensor.

FIG. 42 is a diagram illustrating an example of a head band mounted with a sensor.

FIG. 43 is a diagram illustrating an example of a headphone mounted with a sensor.

FIG. 44 is a diagram illustrating an example of an earphone mounted with a sensor.

FIG. 45 is a diagram illustrating an example of a watch mounted with a sensor.

FIG. 46 is a diagram illustrating an example of glasses mounted with a sensor.

FIG. 47 is a diagram illustrating an example of a schematic configuration of an information processor according to a fifth embodiment of the present disclosure.

FIG. 48 is a diagram illustrating an example of a relationship between a task difference in pnn50 of a pulse wave and the accuracy rate.

FIG. 49 is a diagram illustrating an example of a relationship between a task difference in dispersion of the pnn50 of the pulse wave and the accuracy rate.

FIG. 50 is a diagram illustrating an example of a relationship between a task difference in power of the pnn50 of a pulse wave in the low-frequency band and the accuracy rate.

FIG. 51 is a diagram illustrating an example of a relationship between a task difference in rmssd of the pulse wave and the accuracy rate.

FIG. 52 is a diagram illustrating an example of a relationship between a task difference in dispersion of the rmssd of the pulse wave and the accuracy rate.

FIG. 53 is a diagram illustrating an example of a relationship between a task difference in power of the rmssd of the pulse wave in the low-frequency band and the accuracy rate.

FIG. 54 is a diagram illustrating an example of a relationship between a task difference in dispersion of the number of SCRs of emotional sweating and the accuracy rate.

FIG. 55 is a diagram illustrating an example of a relationship between a task difference in the number of the SCRs of the emotional sweating and the accuracy rate.

FIG. 56 is a diagram illustrating a modification example of the schematic configuration of the information processor in FIG. 25.

FIG. 57 is a diagram illustrating a modification example of the schematic configuration of the information processor in FIG. 28.

FIG. 58 is a diagram illustrating a modification example of the schematic configuration of the information processor in FIG. 30.

FIG. 59 is a diagram illustrating a modification example of the schematic configuration of the information processor in FIG. 47.

FIG. 60 is a diagram illustrating an example in which some of functions of the information processor illustrated in FIG. 14 are provided in a server apparatus.

FIG. 61 is a diagram illustrating an example in which some of functions of the information processor illustrated in FIG. 25 are provided in a server apparatus.

FIG. 62 is a diagram illustrating an example in which some of functions of the information processor illustrated in FIG. 28 are provided in a server apparatus.

FIG. 63 is a diagram illustrating an example in which some of functions of the information processor illustrated in FIG. 30 are provided in a server apparatus.

FIG. 64 is a diagram illustrating an example in which some of functions of the information processor illustrated in FIG. 56 are provided in a server apparatus.

FIG. 65 is a diagram illustrating a modification example of a schematic configuration of an information processing system in FIG. 57.

FIG. 66 is a diagram illustrating a modification example of a schematic configuration of an information processing system in FIG. 58.

FIG. 67 is a diagram illustrating game data that is replaceable with question data of any of FIGS. 14, 17, 19, 28, 30, 47, and 56 to 66.

FIG. 68 is a diagram illustrating a modification example of the information processor in any of FIGS. 14, 17, 19, 22, and 38.

FIG. 69 is a diagram illustrating a modification example of the information processor in any of FIGS. 25, 28, 30, and 35.

FIG. 70 is a diagram illustrating a modification example of the information processor in any of FIGS. 56 to 59.

FIG. 71 is a diagram illustrating a modification example of the information processor in FIG. 47.

FIG. 72 is a diagram illustrating a modification example of the information processor in FIG. 47.

FIG. 73 is a diagram illustrating a modification example of the information processor in FIG. 60.

FIG. 74 is a diagram illustrating a modification example of the information processor in any of FIGS. 61 to 63.

FIG. 75 is a diagram illustrating a modification example of the information processor in any of FIGS. 64 to 66.

FIG. 76 is a diagram illustrating an example of a relationship between a task difference in a median value of reaction times and the accuracy rate.

FIG. 77 is a diagram illustrating an example of a relationship between the arousal level and the accuracy rate.

### MODES FOR CARRYING OUT THE INVENTION

Hereinafter, description is given in detail of embodiments for carrying out the present disclosure with reference to the drawings. It is to be noted that the description is given in the following order.

### 1. Concerning Control of Cognitive Capacity of Present Disclosure (FIGS. 1 to 13)

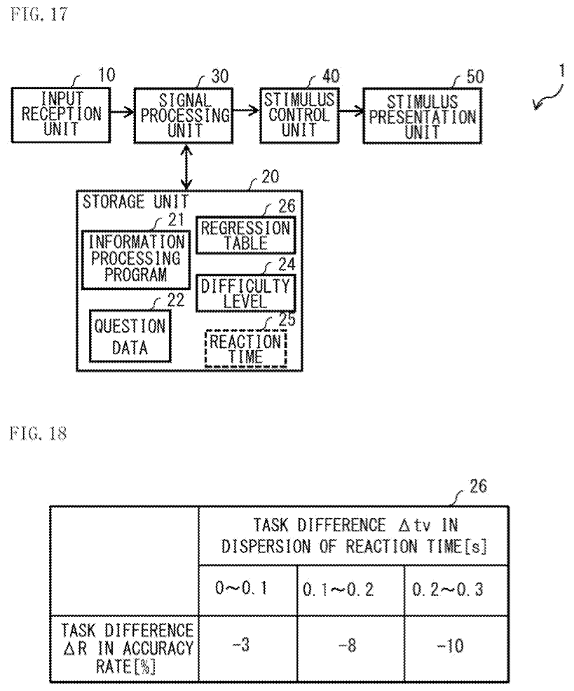

### 2. First Embodiment (FIGS. 14 to 16)

An example of utilizing dispersion of reaction times to derive a cognitive capacity of a user

### 3. Modification Examples of First Embodiment

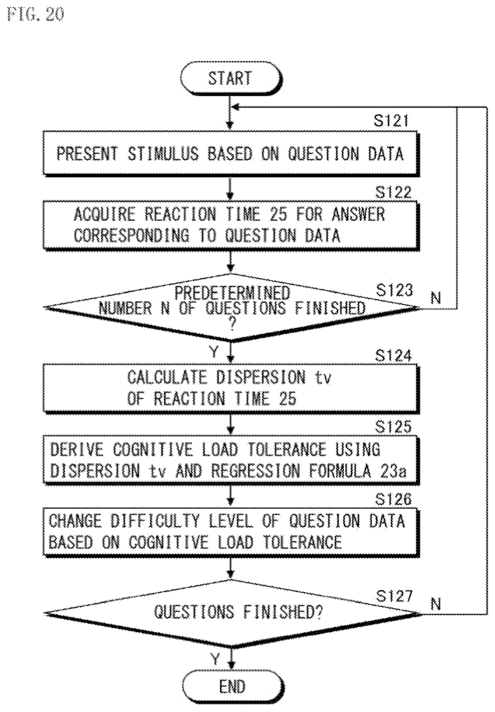

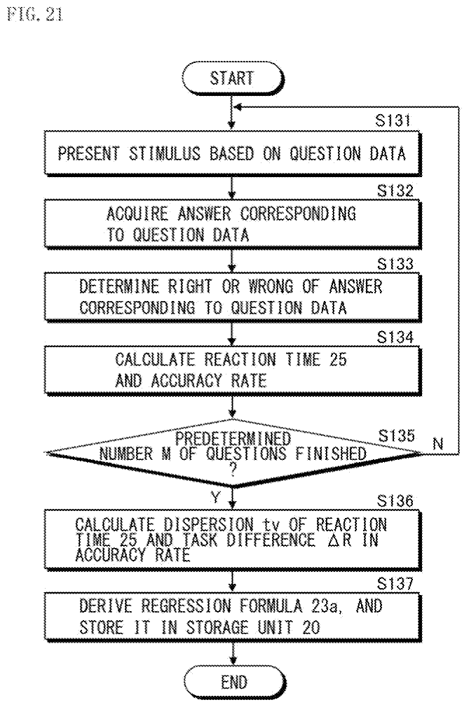

Modification Example A: an example of using a regression table instead of a regression formula (FIGS. 17 and 18)

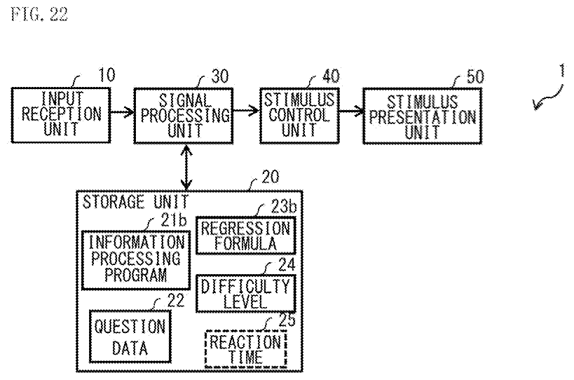

Modification Example B: an example of using another regression formula (FIGS. 19 to 21)

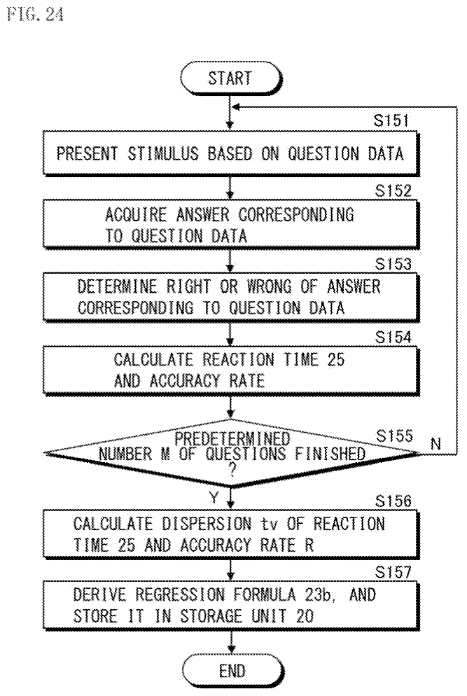

Modification Example C: an example of using another regression formula (FIGS. 22 to 24)

Modification Example D: an example of deriving a cognitive capacity of a group

### 4. Second Embodiment (FIGS. 25 to 27)

An example of utilizing a fluctuation in a slow brain wave to derive a cognitive capacity of a user

### 5. Modification Examples of Second Embodiment

An example of using a regression table instead of a regression formula

An example of deriving a cognitive capacity of a group

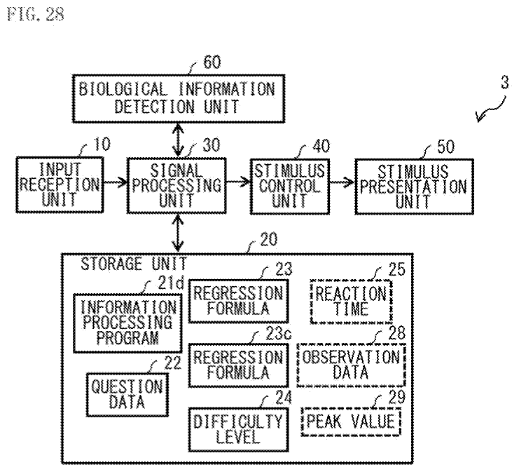

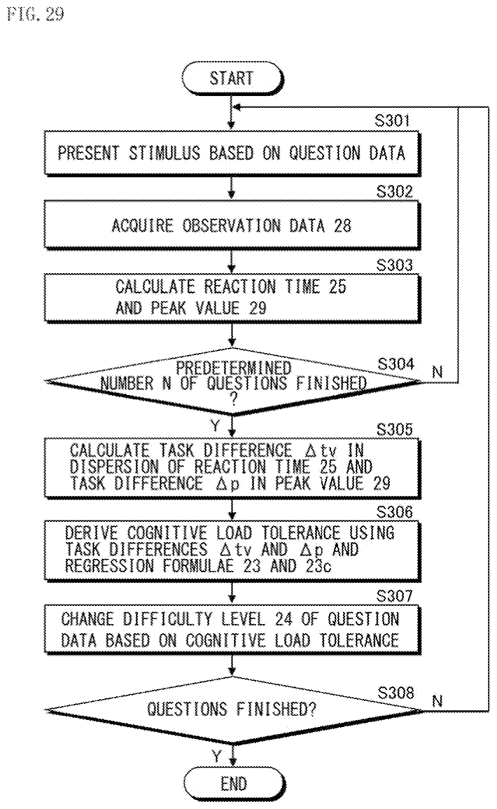

### 6. Third Embodiment (FIGS. 28 and 29)

An example of utilizing dispersion of reaction times and a fluctuation in a slow brain wave to derive a cognitive capacity of a user

### 7. Modification Example of Third Embodiment

An example of deriving a cognitive capacity of a group

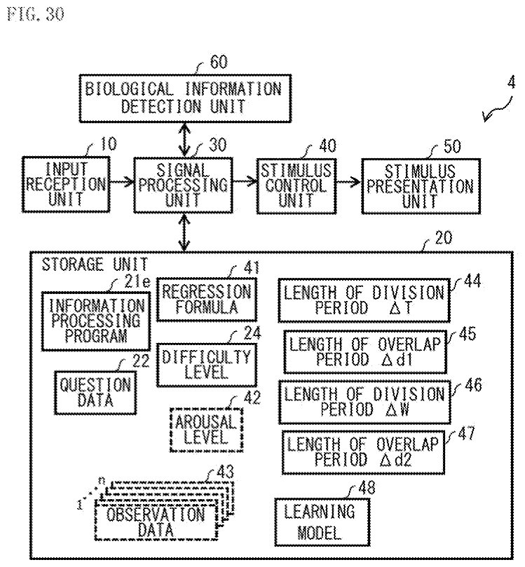

### 8. Fourth Embodiment (FIGS. 30 to 34)

An example of utilizing a learning model that derives an arousal level to derive a cognitive capacity of a user

### 9. Modification Examples of Fourth Embodiment

Modification Example E: an example of using another regression formula (FIGS. 35 to 37)

Modification Example F: an example of deriving a cognitive capacity of a group

### 10. Modification Example of First Embodiment

Modification Example G: an example of using another regression formula (FIGS. 38 to 40)

### 11. Concerning Biological Information Enabling Control of Cognitive Capacity of Present Disclosure (FIGS. 41 to 46)

### 12. Fifth Embodiment

An example of utilizing a pulse wave, an electrocardiogram, a blood flow, and emotional sweating to derive a cognitive capacity of a user (FIGS. 47 to 55)

### 13. Modification Example of Fifth Embodiment

An example of deriving a cognitive capacity of a group

### 14. Modification Examples of First to Fifth Embodiments

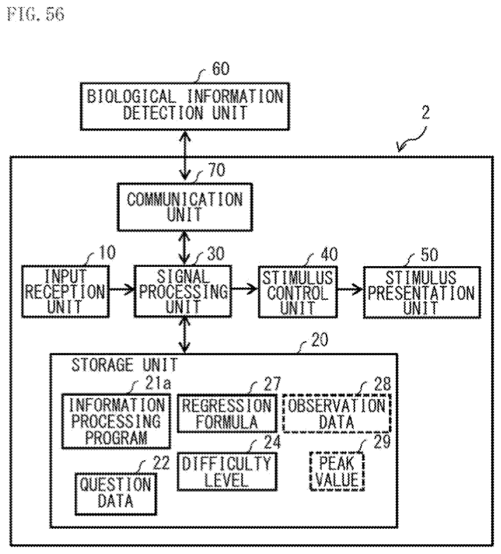

Modification Example H: an example in which a brain wave detection unit is provided separately (FIGS. 56 to 59)

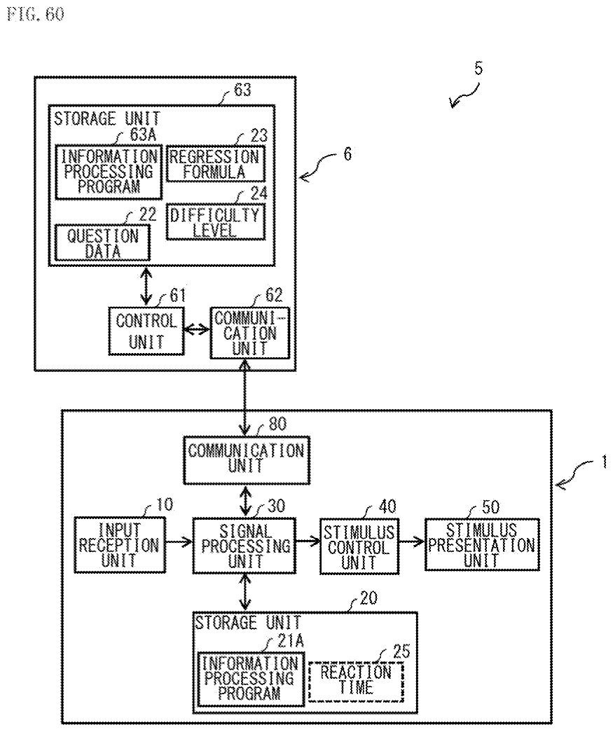

Modification Examples I to O: an example in which a server apparatus derives a cognitive capacity (FIGS. 60 to 66)

Modification Examples P and Q: an example in which the present disclosure is applied to game data (FIG. 67)

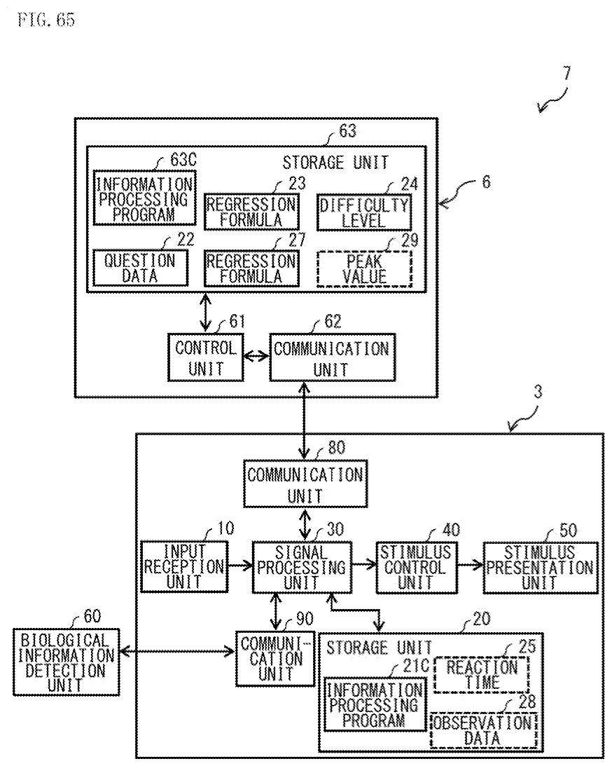

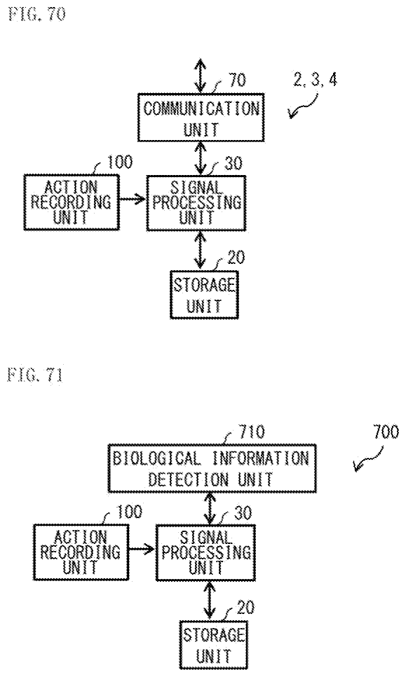

Modification Example R: an example of recording an action of a user (FIGS. 68 to 75)

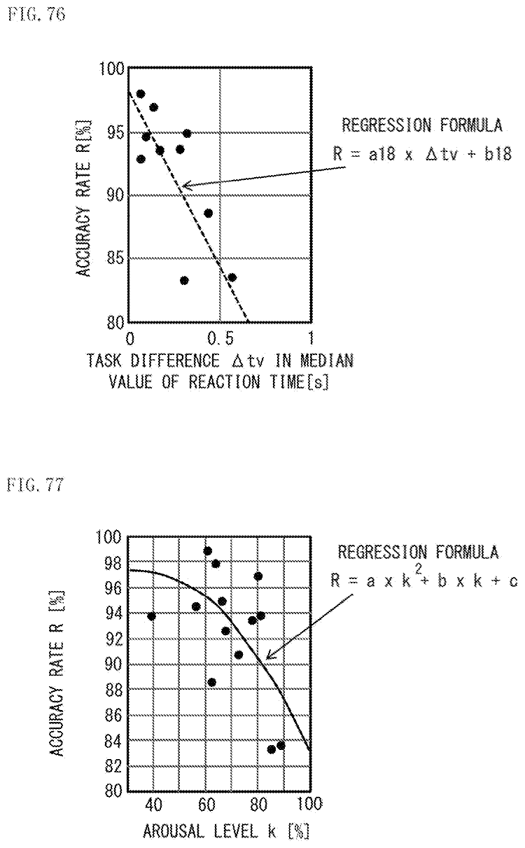

An example of using another regression formula (FIGS. 76 and 77)

### 1. Concerning Control of Cognitive Capacity of Present Disclosure

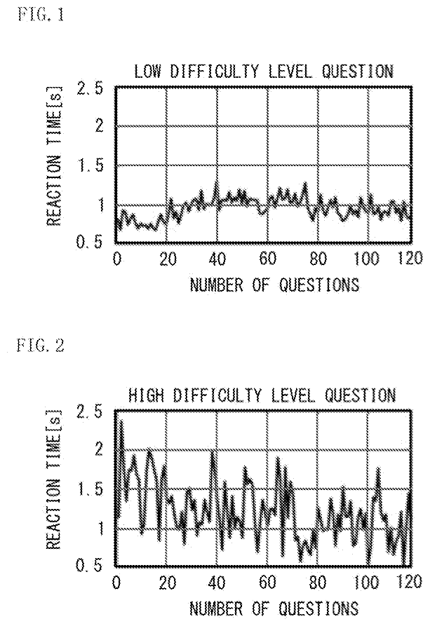

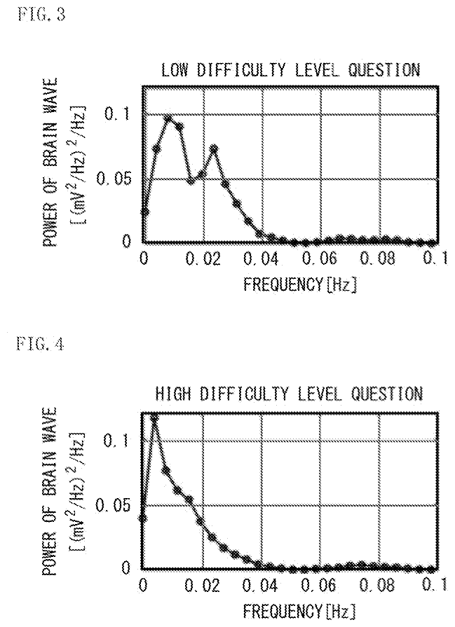

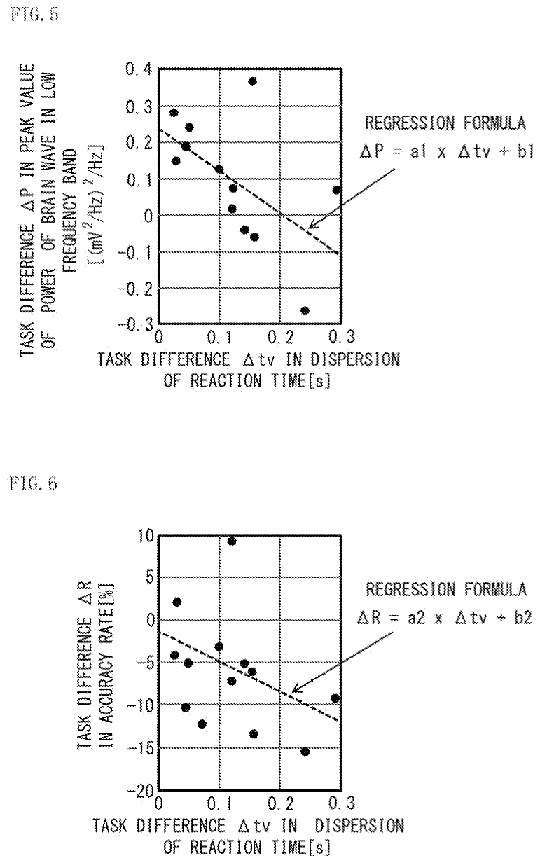

FIGS. 1 and 2 each illustrate, by way of a graph, time (reaction time) required for a user to answer when the user solves a large number of questions in succession. FIG. 1 illustrates a graph at the time of solving questions of a relatively low difficulty level, and FIG. 2 illustrates a graph at the time of solving questions of a relatively high difficulty level. FIG. 3 illustrates a power spectrum density obtained by performing FFT (Fast Fourier Transform) on observation data of a brain wave (α-wave) of the user at the time when the user solves a large number of low difficulty level questions in succession. FIG. 4 illustrates a power spectrum density obtained by performing FFT on observation data of a brain wave (α-wave) of the user at the time when the user solves a large number of high difficulty level questions in succession. FIGS. 3 and 4 each illustrate a graph obtained by measuring a brain wave (α-wave) at a segment of about 20 seconds and performing FFT using an analysis window of about 200 seconds.

It is appreciated from FIGS. 1 and 2 that not only reaction time becomes longer, but also dispersion of the reaction times becomes larger at the time of solving high difficulty level questions, as compared with the time of solving low difficulty level questions. It is appreciated from FIGS. 3 and 4 that power of a brain wave (α-wave) around 0.01 Hz is larger and the power of a brain wave (α-wave) around 0.02 to 0.04 is smaller at the time of solving the high difficulty level questions, as compared with the time of solving the low difficulty level questions. As used herein, the power of the brain wave (α-wave) around 0.01 Hz is appropriately referred to as a “fluctuation in a slow (low-frequency band) brain wave (α-wave)”.

FIG. 5 illustrates an example of a relationship between a task difference Δtv [s] and a task difference ΔP [mV²/Hz)²/Hz]. The task difference Δtv [s] is a task difference in dispersion (75% percentile-25% percentile) of reaction times of the user between the time of solving the high difficulty level questions and the time of solving the low difficulty level questions. The task difference ΔP [mV²/Hz)²/Hz] is a task difference in a peak value of power of the slow brain wave (α-wave) of the user between the time of solving the high difficulty level questions and the time of solving the low difficulty level questions. The task difference Δtv [s] is obtained by subtracting dispersion of reaction times of the user at the time of solving lower-high difficulty level questions from dispersion of reaction times of the user at the time of solving the high difficulty level questions. The task difference ΔP is obtained by subtracting a peak value of the power of the slow brain wave (α-wave) of the user at the time of solving the lower-high difficulty level questions from a peak value of the power of the slow brain wave (α-wave) of the user at the time of solving the high difficulty level questions.

FIG. 6 illustrates an example of a relationship between the task difference Δtv [s] and a task difference ΔR [%]. The task difference Δtv [s] is the task difference in the dispersion (75% percentile-25% percentile) of the reaction times of the user between the time of solving the high difficulty level questions and the time of solving the low difficulty level questions. The task difference ΔR [%] is a task difference in an accuracy rate for questions between the time of solving the high difficulty level questions and the time of solving the low difficulty level questions. The task difference ΔR is obtained by subtracting the accuracy rate at the time of solving the lower-high difficulty level questions from the accuracy rate at the time of solving the high difficulty level questions.

In FIGS. 5 and 6, data for respective users are plotted, and features of the entirety of users are represented by a regression formula (regression line). In FIG. 5, the regression formula is represented by ΔP=a1×Δtv+b1; in FIG. 6, the regression formula is represented by ΔR=a2×Δtv+b2.

A small task difference Δtv in the dispersion of the reaction times means that the difference in the dispersion of the reaction times between the time of solving the high difficulty level questions and the time of solving the low difficulty level questions is small. It can be said that a user who has obtained such a result tends to be able to solve questions in a certain range of reaction time regardless of the difficulty level of the questions. Meanwhile, a large task difference Δtv in the dispersion of the reaction times means that the difference in the dispersion of the reaction times between the time of solving the high difficulty level questions and the time of solving the low difficulty level questions is large. It can be said that a user who has obtained such a result tends to have a large variation in time periods for solving questions as the difficulty level of the questions becomes high.

It is appreciated from FIG. 5 that, when the task difference Δtv in the dispersion of the reaction times is small, the task difference ΔP in the peak value of the power of the slow brain wave (α-wave) becomes large in a plus direction, and that, when the task difference Δtv in the dispersion of the reaction times is large, the task difference ΔP in the peak value of the power of the slow brain wave (α-wave) becomes small. It is appreciated from the above that a person who is able to answer even difficult questions within the same degree of reaction time as that for simple questions has a tendency in which the task difference ΔP in the peak value of the power of the slow brain wave (α-wave) becomes large in the plus direction. Conversely, it is appreciated that a person who has large dispersion of reaction times for difficult questions has a tendency in which the task difference ΔP in the peak value of the power of the slow brain wave (α-wave) does not vary so much regardless of the difficulty level of the questions.

It is appreciated from FIG. 6 that, when the task difference Δtv in the dispersion of the reaction times is large, the task difference ΔR in the accuracy rate for questions becomes large in a minus direction, and that, when the task difference Δtv in the dispersion of the reaction times is small, the task difference ΔR in the accuracy rate for questions becomes small. It is appreciated from the above that a person who has large dispersion of the reaction times for difficult questions has a tendency in which the task difference ΔR in the accuracy rate becomes large in the minus direction (i.e., the accuracy rate for difficult questions is lowered). Conversely, it is appreciated that a person who has small dispersion of the reaction times even for difficult questions has a tendency in which the task difference ΔR in the accuracy rate becomes small (i.e., is able to answer accurately even for difficult questions to the same degree as for simple questions).

It can be inferred from the above that, when the task difference Δtv in the dispersion of the reaction times is large, a cognitive capacity (cognitive resource) of the user is lower than a predetermined standard. The cognitive capacity refers to a capability that includes an execution function, execution efficiency, a working memory, and the like, and is academically called a cognitive resource. In the present disclosure, the cognitive capacity is also called cognitive load tolerance.

In addition, it can be inferred that, when the task difference Δtv in the dispersion of the reaction times is small, the cognitive capacity of the user is higher than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δtv in the dispersion of the reaction times is large, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δtv in the dispersion of the reaction times is small, raising the difficulty level of the questions may possibly bring the cognitive capacity of the user closer to the predetermined standard.

It is appreciated from the above that using the task difference Δtv in the dispersion of the reaction times and the regression formula in FIG. 5 or 6 makes it possible to control the cognitive capacity of the user.

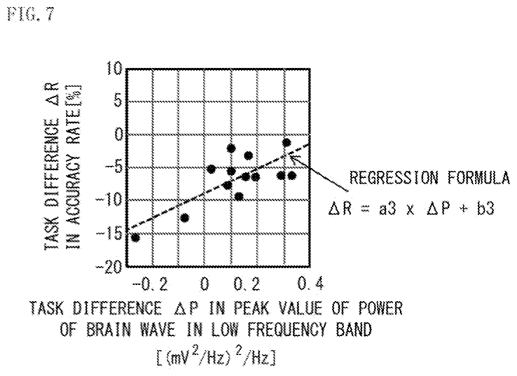

FIG. 7 illustrates an example of a relationship between the task difference ΔP [(mV²/Hz)²/Hz] and the task difference ΔR [%]. The task difference ΔP [(mV²/Hz)²/Hz] is the task difference in the peak value of the power of the slow brain wave (α-wave) of the user between the time of solving the high difficulty level questions and the time of solving the low difficulty level questions. The task difference ΔR [%] is the task difference in the accuracy rate for questions between the time of solving the high difficulty level questions and the time of solving the low difficulty level questions. In FIG. 7, data for respective users are plotted, and features of the entirety of users are represented by a regression formula (regression line). In FIG. 7, the regression formula is represented by ΔR=a3×ΔP+b3.

It is appreciated from FIG. 7 that, when the task difference ΔP in the peak value of the power of the slow brain wave (α-wave) is small (when being around zero), the task difference ΔR in the accuracy rate becomes large in the minus direction (i.e., the accuracy rate for difficult questions is lowered). Conversely, it is appreciated that, when the task difference ΔP in the peak value of the power of the slow brain wave (α-wave) is large in the plus direction (e.g., around 0.4), the task difference ΔR in the accuracy rate becomes small (i.e., it is possible to answer accurately even for difficult questions to the same degree as for simple questions).

It can be inferred from the above that, when the task difference ΔP in the peak value of the power of the slow brain wave (α-wave) is small, the cognitive capacity of the user is lower than a predetermined standard. In addition, it can be inferred that, when the task difference ΔP in the peak value of the power of the slow brain wave (α-wave) is large in the plus direction, the cognitive capacity of the user is higher than the predetermined standard.

In a case where the cognitive capacity of the user is lower than the predetermined standard, lowering the difficulty level of the questions may possibly bring the cognitive capacity of the user closer to the predetermined standard. That is, the task difference ΔP in the peak value of the power of the slow brain wave (α-wave) is small, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. That is, in a case where the task difference ΔP in the peak value of the power of the slow brain wave (α-wave) is large in the plus direction, raising the difficulty level of the questions may possibly bring the cognitive capacity of the user closer to the predetermined standard.

It is appreciated from the above that using the task difference ΔP in the peak value of the power of the slow brain wave (α-wave) and the regression formula in FIG. 7 makes it possible to control the cognitive capacity of the user.

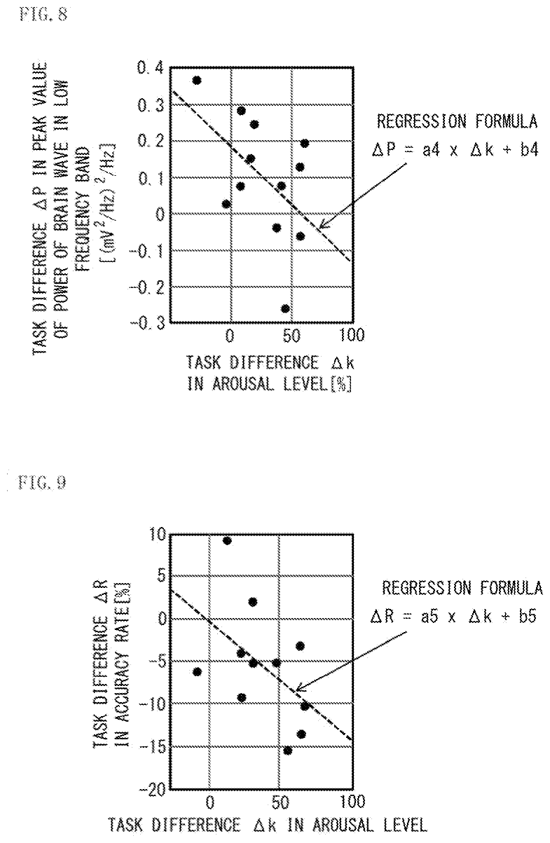

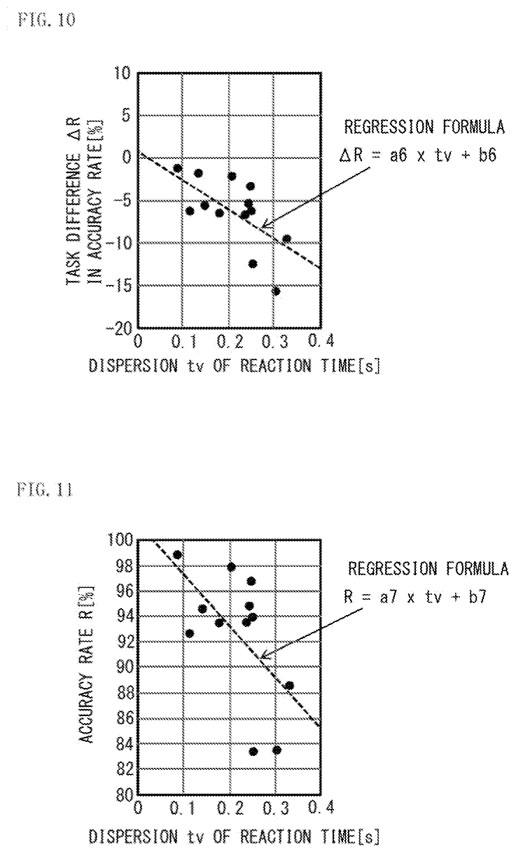

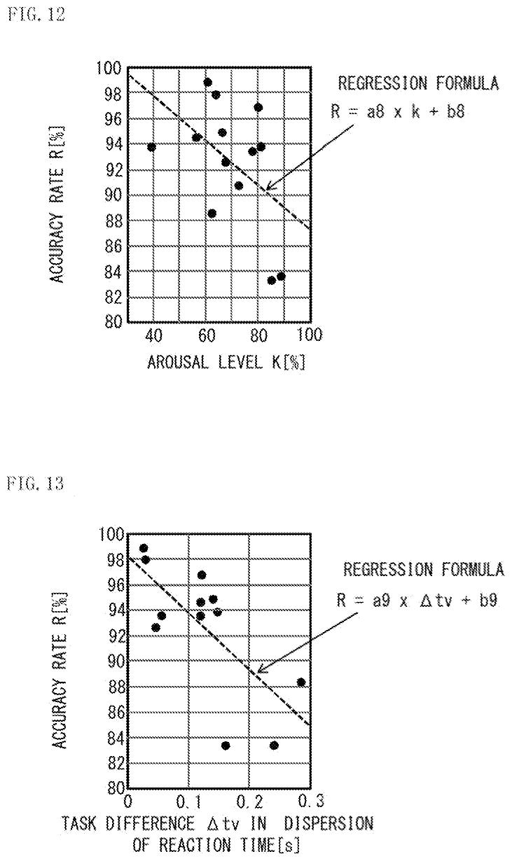

FIG. 8 illustrates an example of a relationship between a task difference Δk [%] and the task difference ΔP [mV²/Hz)²/Hz]. The task difference Δk [%] is a task difference in an arousal level of the user between the time of solving the high difficulty level questions and the time of solving the low difficulty level questions. The task difference ΔP [mV/Hz)²/Hz] is the task difference in the peak value of the power of the slow brain wave (α-wave) of the user between the time of solving the high difficulty level questions and the time of solving the low difficulty level questions. FIG. 9 illustrates an example of a relationship between the task difference Δk [%] and the task difference ΔR [%]. The task difference Δk [%] is the task difference in the arousal level of the user between the time of solving the high difficulty level questions and the time of solving the low difficulty level questions. The task difference ΔR [%] is the task difference in the accuracy rate for questions between the time of solving the high difficulty level questions and the time of solving the low difficulty level questions. The task difference Δk [%] is obtained by subtracting the arousal level of the user at the time of solving the lower-high difficulty level questions from the arousal level of the user at the time of solving the high difficulty level questions. The arousal level is obtained by utilizing an arousal level estimation model described later.

In FIGS. 8 and 9, data for respective users are plotted, and features of the entirety of users are represented by a regression formula (regression line). In FIG. 8, the regression formula is represented by ΔP=a4×Δk+b4; in FIG. 9, the regression formula is represented by ΔR=a5×Δk+b5.

A small task difference Δk in the arousal level means that the difference in the arousal level between the time of solving the high difficulty level questions and the time of solving the low difficulty level questions is small. It can be said that a user who has obtained such a result tends to be able to solve questions in a certain arousal level regardless of the difficulty level of the questions. Meanwhile, a large task difference Δk in the arousal level means that the difference in the arousal level between the time of solving the high difficulty level questions and the time of solving the low difficulty level questions is large. It can be said that a user who has obtained such a result tends to have a high arousal level as the difficulty level of the questions becomes high.

It is appreciated from FIG. 8 that, when the task difference Δk in the arousal level is small, the task difference ΔP in the peak value of the power of the slow brain wave (α-wave) becomes large in the plus direction, and that, when the task difference Δk in the arousal level is large, the task difference ΔP in the peak value of the power of the slow brain wave (α-wave) becomes small. It is appreciated from the above that a person who is able to answer even difficult questions in the same degree of arousal level as that for simple questions has a tendency in which the task difference ΔP in the peak value of the power of the slow brain wave (α-wave) becomes large in the plus direction. Conversely, it is appreciated that a person who has a high arousal level for difficult questions has a tendency in which the task difference ΔP in the peak value of the power of the slow brain wave (α-wave) does not vary so much regardless of the difficulty level of the questions.

It is appreciated from FIG. 9 that, when the task difference Δk in the arousal level is large, the task difference ΔR in the accuracy rate for questions becomes large in the minus direction, and that, when the task difference Δk in the arousal level is small, the task difference ΔR in the accuracy rate for questions becomes small. It is appreciated from the above that a person who has a high arousal level for difficult questions has a tendency in which the task difference ΔR in the accuracy rate becomes large in the minus direction (i.e., the accuracy rate for the difficult questions is lowered). Conversely, it is appreciated that a person who has a small arousal level even for difficult questions has a tendency in which the task difference ΔR in the accuracy rate becomes small (i.e., is able to answer accurately even for difficult questions to the same degree as for simple questions).

It can be inferred from the above that, when the task difference Δk in the arousal level is large, the cognitive capacity of the user is lower than a predetermined standard. In addition, it can be inferred that, when the task difference Δk in the arousal level is small, the cognitive capacity of the user is higher than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δk in the arousal level is large, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δk in the arousal level is small, raising the difficulty level of the questions may possibly bring the cognitive capacity of the user closer to the predetermined standard.

It is appreciated from the above that using the task difference Δk in the arousal level and the regression formula in FIG. 8 or 9 makes it possible to control the cognitive capacity of the user.

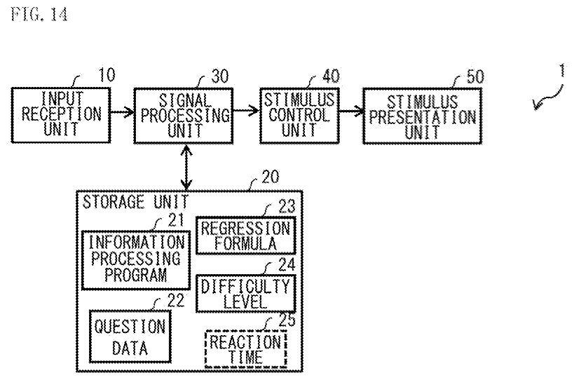

FIG. 10 illustrates an example of a relationship between dispersion (75% percentile-25% percentile) tv [s] and the task difference ΔR [%]. The dispersion (75% percentile-25% percentile) tv [s] is dispersion of the reaction times of the user at the time of solving the high difficulty level questions. The task difference ΔR [%] is the task difference in the accuracy rate for questions between the time of solving the high difficulty level questions and the time of solving the low difficulty level questions. The task difference ΔR is obtained by subtracting the accuracy rate at the time of solving the lower-high difficulty level questions from the accuracy rate at the time of solving the high difficulty level questions. In FIG. 10, data for respective users are plotted, and features of the entirety of users are represented by a regression formula (regression line). In FIG. 10, the regression formula is represented by ΔR=a6×tv+b6.

Small dispersion tv of the reaction times means that the high difficulty level questions have been solved within a substantially constant time period. It can be said that the difficulty level of the questions is not so high for a user who has obtained such a result. Meanwhile, large dispersion tv of the reaction times means large dispersion of time periods for solving the high difficulty level questions. It can be said that the difficulty level of the questions is relatively high for a user who has obtained such a result.

It is appreciated from FIG. 10 that, when the dispersion tv of the reaction times is large, the task difference ΔR in the accuracy rate for questions becomes large in the minus direction, and that, when the dispersion tv of the reaction times is small, the task difference ΔR in the accuracy rate for questions becomes small. It is appreciated from the above that a person who has large dispersion of the reaction times for difficult questions has a tendency in which the task difference ΔR in the accuracy rate becomes large in the minus direction (i.e., the accuracy rate for the difficult questions is lowered). Conversely, it is appreciated that a person who has small dispersion of the reaction times even for difficult questions has a tendency in which the task difference ΔR in the accuracy rate becomes small (i.e., is able to answer accurately even for difficult questions to the same degree as for simple questions).

It can be inferred from the above that, when the dispersion tv of the reaction times is large, the cognitive capacity of the user is lower than a predetermined standard. In addition, it can be inferred that, when the dispersion tv of the reaction times is small, the cognitive capacity of the user is higher than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the dispersion tv of the reaction times is large, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the dispersion tv of the reaction times is small, raising the difficulty level of the questions may possibly bring the cognitive capacity of the user closer to the predetermined standard.

It is appreciated from the above that using the dispersion tv of the reaction times and the regression formula in FIG. 10 makes it possible to control the cognitive capacity of the user.

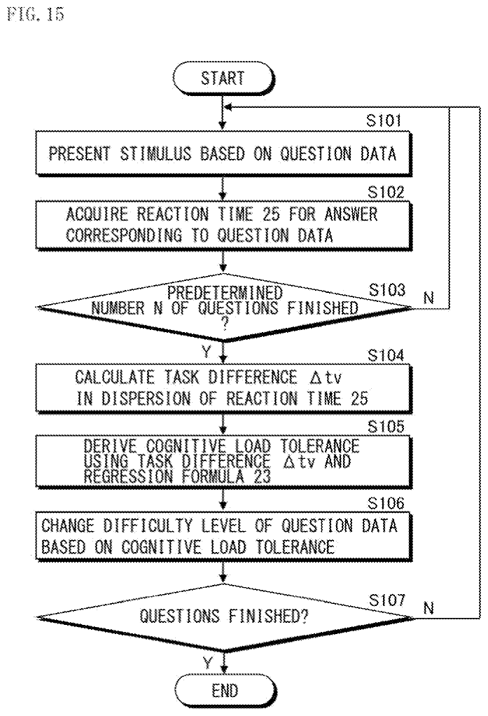

FIG. 11 illustrates an example of a relationship between the dispersion (75% percentile-25% percentile) tv [s] and an accuracy rate R [%]. The dispersion (75% percentile-25% percentile) tv [s] is the dispersion of the reaction times of the user at the time of solving the high difficulty level questions. The accuracy rate R [%] is an accuracy rate for questions at the time of solving the high difficulty level questions. In FIG. 11, data for respective users are plotted, and features of the entirety of users are represented by a regression formula (regression line). In FIG. 11, the regression formula is represented by R=a7×tv+b7.

Small dispersion tv of the reaction times means that the high difficulty level questions have been solved within a substantially constant time period. It can be said that the difficulty level of the questions is not so high for a user who has obtained such a result. Meanwhile, large dispersion tv of the reaction times means large dispersion of time periods for solving the high difficulty level questions. It can be said that the difficulty level of the questions is relatively high for a user who has obtained such a result.

It is appreciated from FIG. 11 that, when the dispersion tv of the reaction times is large, the accuracy rate R for the high difficulty level questions becomes low, and that, when the dispersion tv of the reaction times is small, the accuracy rate R for the high difficulty level questions becomes large. It is appreciated from the above that a person who has large dispersion of the reaction times for difficult questions tends to have a lower accuracy rate R for the difficult questions. Conversely, it is appreciated that a person who has small dispersion of the reaction times even for difficult questions tends to have a higher accuracy rate R (i.e., be able to answer accurately even for difficult questions to the same degree as for simple questions).

It can be inferred from the above that, when the dispersion tv of the reaction times is large, the cognitive capacity of the user is lower than a predetermined standard. In addition, it can be inferred that, when the dispersion tv of the reaction times is small, the cognitive capacity of the user is higher than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the dispersion tv of the reaction times is large, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the dispersion tv of the reaction times is small, raising the difficulty level of the questions may possibly bring the cognitive capacity of the user closer to the predetermined standard.

It is appreciated from the above that using the dispersion tv of the reaction times and the regression formula in FIG. 11 makes it possible to control the cognitive capacity of the user.

FIG. 12 illustrates an example of a relationship between an arousal level k [%] and the accuracy rate R [%]. The arousal level k [%] is an arousal level of the user at the time of solving the high difficulty level questions. The accuracy rate R [%] is the accuracy rate for questions at the time of solving the high difficulty level questions. In FIG. 12, data for respective users are plotted, and features of the entirety of users are represented by a regression formula (regression line). In FIG. 12, the regression formula is represented by R=a8×k+b8.

A small arousal level k means that the arousal level at the time of solving the high difficulty level questions is small. It can be said that the difficulty level of the questions is not so high for a user who has obtained such a result. Meanwhile, a large arousal level k means that the arousal level at the time of solving the high difficulty level questions is large. It can be said that the difficulty level of the questions is relatively high for a user who has obtained such a result.

It is appreciated from FIG. 12 that, when the arousal level k is large, the accuracy rate R for the questions becomes low, and that, when the arousal level k is small, the accuracy rate for the questions becomes high. It is appreciated from the above that a person who has a high arousal level for difficult questions tends to have lower accuracy rate R (i.e., have a lower accuracy rate for the difficult questions). Conversely, it is appreciated that a person who has a small arousal level even for difficult questions tends to have higher accuracy rate R (i.e., be able to answer accurately even for difficult questions to the same degree as for simple questions).

It can be inferred from the above that, when the arousal level k is large, the cognitive capacity of the user is lower than a predetermined standard. In addition, it can be inferred that, when the arousal level k is small, the cognitive capacity of the user is higher than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the arousal level k is large, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the arousal level k is small, raising the difficulty level of the questions may possibly bring the cognitive capacity of the user closer to the predetermined standard.

It is appreciated from the above that using the arousal level k and the regression formula in FIG. 12 makes it possible to control the cognitive capacity of the user.

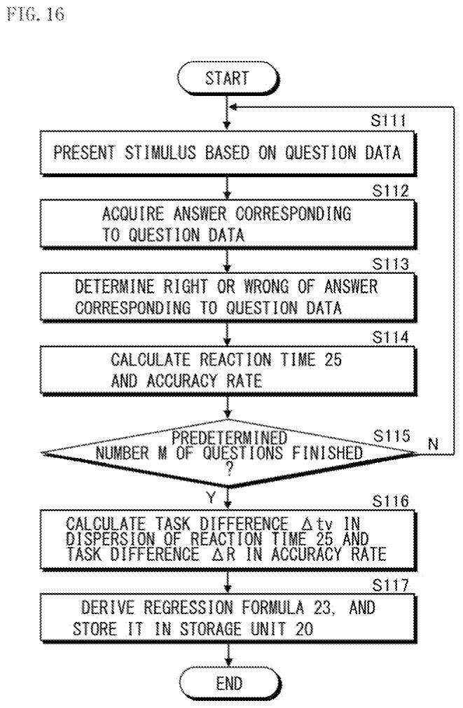

FIG. 13 illustrates an example of a relationship between the task difference Δtv [s] and the accuracy rate R [%]. The task difference Δtv [s] is the task difference in the dispersion (75% percentile−25% percentile) of the reaction times of the user between the time of solving the high difficulty level questions and the time of solving the low difficulty level questions. The accuracy rate R [%] is the accuracy rate for the questions at the time of solving the high difficulty level questions. In FIG. 13, data for respective users are plotted, and features of the entirety of users are represented by a regression formula (regression line). In FIG. 13, the regression formula is represented by R=a9×Δtv+b9.

A small task difference Δtv in the dispersion of the reaction times means that the difference in the dispersion of the reaction times between the time of solving the high difficulty level questions and the time of solving the low difficulty level questions is small. It can be said that a user who has obtained such a result tends to be able to solve questions in a certain range of reaction time regardless of the difficulty level of the questions. Meanwhile, a large task difference Δtv in the dispersion of the reaction times means that the difference in the dispersion of the reaction times between the time of solving the high difficulty level questions and the time of solving the low difficulty level questions is large. It can be said that a user who has obtained such a result tends to have a large variation in time periods for solving questions as the difficulty level of the questions becomes high.

It is appreciated from FIG. 13 that, when the task difference Δtv in the dispersion of the reaction times is large, the accuracy rate R for questions becomes low, and that, when the task difference Δtv in the dispersion of the reaction times is small, the accuracy rate R for questions becomes large. It is appreciated from the above that a person who has large dispersion of the reaction times for difficult questions tends to have a lower accuracy rate R (i.e., the accuracy rate for the difficult questions is lowered). Conversely, it is appreciated that a person who has small dispersion of the reaction times even for difficult questions tends to have a higher accuracy rate R (i.e., be able to answer accurately even for difficult questions to the same degree as for simple questions).

It can be inferred from the above that, when the task difference Δtv in the dispersion of the reaction times is large, the cognitive capacity of the user is lower than a predetermined standard. In addition, it can be inferred that, when the task difference Δtv in the dispersion of the reaction times is small, the cognitive capacity of the user is higher than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δtv in the dispersion of the reaction times is large, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δtv in the dispersion of the reaction times is small, raising the difficulty level of the questions may possibly bring the cognitive capacity of the user closer to the predetermined standard.

It is appreciated from the above that using the task difference Δtv in the dispersion of the reaction times and the regression formula in FIG. 13 makes it possible to control the cognitive capacity of the user.

### 2. First Embodiment

### [Configuration]

Description is given of an information processor 1 according to a first embodiment of the present disclosure. FIG. 14 illustrates an example of a schematic configuration of the information processor 1 according to the present embodiment. The information processor 1 includes an input reception unit 10, a storage unit 20, a signal processing unit 30, a stimulus control unit 40, and a stimulus presentation unit 50. The signal processing unit 30 corresponds to a specific example of each of an “acquisition unit”, a “determination unit”, and a “deriving unit” of the present disclosure. The stimulus presentation unit 50 corresponds to a specific example of a “presentation unit” of the present disclosure.

The input reception unit 10 accepts an input from a user, and outputs it to the signal processing unit 30. Examples of the input from the user include a reaction of the user for a stimulus presented by the stimulus presentation unit 50. For example, in a case where the stimulus presented by the stimulus presentation unit 50 is provision of questions by means of an image (a still image or a moving image), a sound, or light, examples of a reaction of the user may include inputting an answer corresponding to question data 22 (described later) into the input reception unit 10. At this time, the input reception unit 10 receives the input from the user as an answer corresponding to the provided question data 22 (described later), and outputs the received answer to the signal processing unit 30. The input reception unit 10 includes, for example, an input interface such as a keyboard, a mouse, or a touch panel.

The storage unit 20 is, for example, a volatile memory such as a DRAM (Dynamic Random Access Memory), or a non-volatile memory such as an EEPROM (Electrically Erasable Programmable Read-Only Memory) or a flash memory. The storage unit 20 stores an information processing program 21 to control the cognitive capacity of the user, and the question data 22, a regression formula 23, and a difficulty level 24 which are to be used in the information processing program 21. The regression formula 23 corresponds to a specific example of “regression data” of the present disclosure. Further, the storage unit 20 stores a reaction time 25 obtained by processing by the information processing program 21. The information processing program 21 is described in detail later.

The question data 22 includes a plurality of pieces of question data of different difficulty levels. The question data is a question in learning, and corresponds to a specific example of each of a “request” and a “task” of the present disclosure. The question data 22 also includes data on a difficulty level of each of pieces of question data included in the question data 22. The question data 22 may further include correct answer data for each of the pieces of question data. The regression formula 23 is, for example, the regression formula illustrated in FIG. 5 or 6. The reaction time 25 is, for example, time required from provision of a question to inputting of an answer. The reaction time 25 is, for example, time required from presentation of a stimulus by the stimulus presentation unit 50 to reception by the input reception unit 10 of a reaction of a user (e.g., an answer input timing of a user) to the stimulus presented by the stimulus presentation unit 50.

The difficulty level 24 includes, for example, data for setting the difficulty level of questions to be provided to the user, and a table describing a correspondence relationship between the difficulty level of the questions and the cognitive capacity of the user. The setting data included in the difficulty level 24 concerns a plurality of difficulty levels as initial values or a plurality of difficulty levels after having been changed by processing by the information processing program 21. The table included in the difficulty level 24 has difficulty levels set in accordance with the cognitive capacity of the user. For example, the table included in the difficulty level 24 has a plurality of difficulty levels, which are set as difficulty levels corresponding to a cognitive capacity a, in a case where the cognitive capacity of the user is a. The setting of the plurality of difficulty levels as difficulty levels corresponding to the cognitive capacity a enables the information processing program 21 to provide the user with questions of the plurality of difficulty levels.

The signal processing unit 30 is configured by a processor, for example. The signal processing unit 30 executes the information processing program 21 stored in the storage unit 20. Functions of the signal processing unit 30 are implemented, for example, by execution of the information processing program 21 by the signal processing unit 30. For example, the signal processing unit 30 reads question data of the plurality of difficulty levels corresponding to the setting data included in the difficulty level 24 from among the question data 22, and sequentially outputs the read question data of the plurality of difficulty levels to the stimulus control unit 40. When acquiring an answer corresponding to the question data 22 from the input reception unit 10, for example, the signal processing unit 30 derives the reaction time 25 on the basis of an input timing of the acquired answer. For example, when provision of a predetermined number N of questions is completed, the signal processing unit 30 calculates the task difference Δtv in dispersion of the reaction times 25. On the basis of the calculated task difference Δtv and the regression formula 23 read from the storage unit 20, for example, the signal processing unit 30 derives a cognitive capacity. On the basis of the derived cognitive capacity, for example, the signal processing unit 30 determines the difficulty level of questions to be provided subsequently. On the basis of a table included in the difficulty level 24 read from the storage unit 20, for example, the signal processing unit 30 determines the difficulty level of the questions to be provided subsequently. For example, the signal processing unit 30 writes the determined difficulty levels into the setting data of the storage unit 20 to thereby set the difficulty level of the questions to be provided subsequently.

The stimulus control unit 40 generates a control signal to control the stimulus presentation unit 50 on the basis of the question data inputted from the signal processing unit 30. The stimulus control unit 40 outputs the generated control signal to the stimulus presentation unit 50. In a case where the stimulus presentation unit 50 is a display panel, the stimulus control unit 40 generates, as a control signal, an image signal to display the question data inputted from the signal processing unit 30. In a case where the stimulus presentation unit 50 is a sound speaker, the stimulus control unit 40 generates, as a control signal, a sound signal to speak the question data inputted from the signal processing unit 30. In a case where the stimulus presentation unit 50 is a light-emitting device, the stimulus control unit 40 generates, as a control signal, a light-emitting control signal corresponding to the question data inputted from the signal processing unit 30.

The stimulus presentation unit 50 presents a stimulus to the user on the basis of the control signal inputted from the stimulus control unit 40. In a case where the stimulus presentation unit 50 is a display panel, the stimulus presentation unit 50 presents, to the user, an image including a plurality of pieces of question data of different difficulty levels on the basis of the image signal inputted from the stimulus control unit 40. In a case where the stimulus presentation unit 50 is a sound speaker, the stimulus presentation unit 50 presents, to the user, a sound to speak the plurality of pieces of question data of different difficulty levels on the basis of the sound signal inputted from the stimulus control unit 40. In a case where the stimulus presentation unit 50 is a light-emitting device, the stimulus presentation unit 50 presents, to the user, light corresponding to the plurality of pieces of question data of different difficulty levels on the basis of the light-emitting control signal inputted from the stimulus control unit 40.

### [Operations]

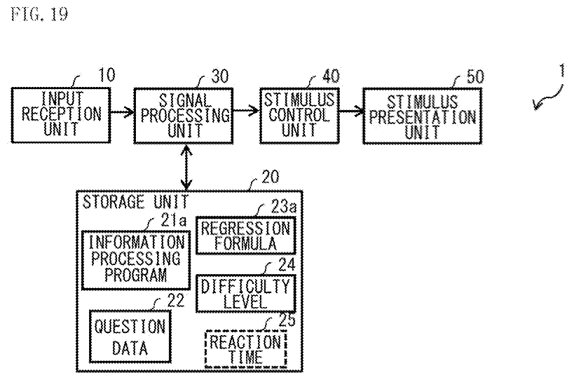

Next, description is given of operations of the information processor 1. FIG. 15 illustrates an example of a procedure to change a difficulty level in the information processor 1.

First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S101). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. When acquiring the answer from the input reception unit 10, the signal processing unit 30 calculates (acquires) the reaction time 25 for the answer corresponding to the question data (step S102).

The signal processing unit 30 executes the above-described series of processing until the provision of the predetermined number N of questions is completed (step S103; N). When the provision of the predetermined number N of questions is completed (step S103; Y), the signal processing unit 30 calculates the task difference Δtv in the dispersion of the reaction times 25 acquired thus far (step S104). The signal processing unit 30 derives a cognitive capacity using the calculated task difference Δtv and the regression formula 23 read from the storage unit 20 (step S105). The signal processing unit 30 determines the difficulty level of questions to be provided subsequently using the table included in the difficulty level 24 read from the storage unit 20. The signal processing unit 30 writes the determined difficulty levels into the setting data of the storage unit 20 to thereby change the difficulty level of questions to be provided subsequently (step S106).

The signal processing unit 30 executes the above-described series of processing until the provision of the predetermined number of questions is completed (step S107; N). When the provision of the predetermined number of questions is completed (step S107; Y), the signal processing unit 30 finishes the provision.

FIG. 16 illustrates an example of a procedure to derive the regression formula 23 in the information processor 1. First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S111). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. The input reception unit 10 acquires the answer corresponding to the question data from the user (step S112). The input reception unit 10 outputs the acquired answer to the signal processing unit 30. When acquiring the answer from the input reception unit 10, the signal processing unit 30 determines right or wrong of the answer corresponding to the question data using correct answer data included in the question data 22 (step S113). The signal processing unit 30 calculates (acquires) the reaction time 25 and the accuracy rate for the answer corresponding to the question data (step S114).

The signal processing unit 30 executes the above-described series of processing until the provision of the predetermined number N of questions is completed (step S105; N). When the provision of the predetermined number N of questions is completed (step S105; Y), the signal processing unit 30 calculates the task difference Δtv in the dispersion of the reaction times 25 acquired thus far and the task difference ΔR in the accuracy rate for answers acquired thus far (step S116). On the basis of the calculated task differences Δtv and ΔR, the signal processing unit 30 derives the regression formula 23, and stores the derived regression formula 23 in the storage unit 20 (step S117).

The information processor 1 may perform the series of procedures to derive the regression formula 23 illustrated in FIG. 16, separately (i.e., in advance) from the series of procedures to change the difficulty levels in the information processor 1 illustrated in FIG. 15. At this time, the user who answers the questions to derive the regression formula 23 and the user who answers the questions in the series of procedures illustrated in FIG. 15 may be the same as or different from each other. It is to be noted that the information processor 1 may perform the series of procedures for deriving the regression formula 23 illustrated in FIG. 16 to be mixed into the series of procedures of steps S101 to S103 illustrated in FIG. 15.

### [Effects]

Next, description is given of effects of the information processor 1.

In the information processor 1 and the information processing program 21 according to the present embodiment, a plurality of pieces of question data to be presented to the user is determined on the basis of the dispersion of the reaction times 25 of the user corresponding to the plurality of pieces of question data. Here, the present discloser has experimentally obtained knowledge that the dispersion of the reaction times 25 varies depending on tasks. It is therefore possible to determine the plurality of pieces of question data to be presented to the user on the basis of the dispersion of the reaction times 25. Thus, it is possible to derive the cognitive capacity regardless of whether or not there is correct reaction time.

In the information processor 1 and the information processing program 21 according to the present embodiment, the cognitive capacity of the user is derived on the basis of the dispersion of the reaction times 25. This makes it possible, on the basis of the derived cognitive capacity, to determine the difficulty levels of the plurality of pieces of question data to be presented to the user and to determine a plurality of pieces of subsequent question data. Thus, it is possible to derive the cognitive capacity regardless of whether or not there is correct reaction time.

In the information processor 1 and the information processing program 21 according to the present embodiment, the cognitive capacity is derived on the basis of the task difference Δtv in the dispersion of the reaction times 25 and the regression formula 23 for the task difference Δtv in the dispersion of the reaction times 25. This makes it possible to derive the cognitive capacity regardless of whether or not there is correct reaction time.

In the information processor 1 and the information processing program 21 according to the present embodiment, the difficulty levels of the plurality of pieces of question data to be provided subsequently are determined by using the table in which the difficulty levels corresponding to the cognitive capacity are set. This makes it possible to set the difficulty levels of the plurality of pieces of question data to be provided subsequently, for example, to bring the cognitive capacity of the user closer to a predetermined standard.

In the information processor 1 and the information processing program 21 according to the present embodiment, the reaction time 25 is derived on the basis of an answer input timing of the user for the plurality of pieces of question data. This makes it possible to derive the cognitive capacity on the basis of the task difference Δtv in the dispersion of the reaction times 25 and the regression formula 23 for the task difference Δtv in the dispersion of the reaction times 25. Thus, it is possible to derive the cognitive capacity regardless of whether or not there is correct reaction time 25.

The information processor 1 according to the present embodiment is provided with the stimulus presentation unit 50 that presents the plurality of pieces of question data. This makes it possible to control presentation timings of respective pieces of question data, thus making it possible to derive the reaction time 25 accurately. As a result, it is possible to derive the cognitive capacity accurately regardless of whether or not there is correct reaction time 25.

### 3. Modification Examples of First Embodiment

### Modification Example A

In the foregoing embodiment, for example, as illustrated in FIG. 17, a regression table 26 may be stored in the storage unit 20, instead of the regression formula 23. In the regression table 26, for example, as illustrated in FIG. 18, a correspondence relationship between the task difference Δtv [s] and the task difference ΔR [%] is set using a table. The task difference Δtv [s] is the task difference in the dispersion (75% percentile−25% percentile) of the reaction times of the user between the time of solving the high difficulty level questions and the time of solving the low difficulty level questions. The task difference ΔR [%] is the task difference in the accuracy rate for questions between the time of solving the high difficulty level questions and the time of solving the low difficulty level questions. In the regression table 26, for example, when the task difference Δtv [s] is within a range of 0 to 0.1, the task difference ΔR [%] in the accuracy rate for questions is −3%; when the task difference Δtv [s] is within a range of 0.1 to 0.2, the task difference ΔR [%] in the accuracy rate for questions is −8%; and when the task difference Δtv [s] is within a range of 0.2 to 0.3, the task difference ΔR [%] in the accuracy rate for questions is −10%.

In the present modification example, the regression table 26 is used instead of the regression formula 23. Also in such a case, it is possible to derive the cognitive capacity regardless of whether or not there is correct reaction time 25.

### Modification Example B

In the foregoing embodiment, for example, as illustrated in FIG. 19, an information processing program 21a and a regression formula 23a may be stored in the storage unit 20, instead of the information processing program 21 and the regression formula 23. The regression formula 23a is, for example, the regression formula illustrated in FIG. 10.

The signal processing unit 30 executes the information processing program 21a stored in the storage unit 20. The functions of the signal processing unit 30 are implemented, for example, by execution of the information processing program 21a by the signal processing unit 30. For example, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. When acquiring an answer corresponding to the question data 22 from the input reception unit 10, for example, the signal processing unit 30 derives the reaction time 25 on the basis of an input timing of the acquired answer. For example, when provision of the predetermined number N of questions is completed, the signal processing unit 30 calculates the dispersion tv of the reaction times 25. On the basis of the calculated dispersion tv and the regression formula 23a read from the storage unit 20, for example, the signal processing unit 30 derives a cognitive capacity. On the basis of the derived cognitive capacity, for example, the signal processing unit 30 determines the difficulty level of questions to be provided subsequently. On the basis of a table included in the difficulty level 24 read from the storage unit 20, for example, the signal processing unit 30 determines the difficulty level of the questions to be provided subsequently. For example, the signal processing unit 30 writes the determined difficulty levels into the setting data of the storage unit 20 to thereby set the difficulty level of the questions to be provided subsequently.

Next, description is given of operations of the information processor 1. FIG. 20 illustrates an example of a procedure to change a difficulty level in the information processor 1.

First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S121). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. When acquiring the answer from the input reception unit 10, the signal processing unit 30 calculates (acquires) the reaction time 25 for the answer corresponding to the question data (step S122).

The signal processing unit 30 executes the above-described series of processing until the provision of the predetermined number N of questions is completed (step S123; N). When the provision of the predetermined number N of questions is completed (step S123; Y), the signal processing unit 30 calculates the dispersion tv of the reaction times 25 acquired thus far (step S124). The signal processing unit 30 derives a cognitive capacity using the calculated dispersion tv and the regression formula 23a read from the storage unit 20 (step S125). The signal processing unit 30 determines the difficulty level of questions to be provided subsequently using the table included in the difficulty level 24 read from the storage unit 20. The signal processing unit 30 writes the determined difficulty levels into the setting data of the storage unit 20 to thereby change the difficulty level of questions to be provided subsequently (step S126).

The signal processing unit 30 executes the above-described series of processing until the provision of the predetermined number of questions is completed (step S127; N). When the provision of the predetermined number of questions is completed (step S127; Y), the signal processing unit 30 finishes the provision.

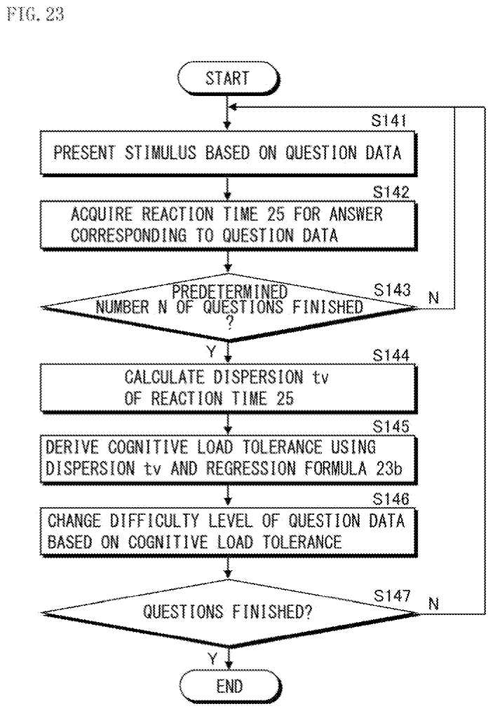

FIG. 21 illustrates an example of a procedure to derive the regression formula 23a in the information processor 1. First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S131). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. The input reception unit 10 acquires the answer corresponding to the question data from the user (step S132). The input reception unit 10 outputs the acquired answer to the signal processing unit 30. When acquiring the answer from the input reception unit 10, the signal processing unit 30 determines right or wrong of the answer corresponding to the question data using correct answer data included in the question data 22 (step S133). The signal processing unit 30 calculates (acquires) the reaction time 25 and the accuracy rate for the answer corresponding to the question data (step S134).

The signal processing unit 30 executes the above-described series of processing until the provision of the predetermined number N of questions is completed (step S135; N). When the provision of the predetermined number N of questions is completed (step S135; Y), the signal processing unit 30 calculates the dispersion tv of the reaction times 25 acquired thus far and the task difference ΔR in the accuracy rate for answers acquired thus far (step S136). On the basis of the calculated dispersion tv and the task difference ΔR, the signal processing unit 30 derives the regression formula 23a, and stores the derived regression formula 23a in the storage unit 20 (step S137).

The information processor 1 may perform the series of procedures to derive the regression formula 23a illustrated in FIG. 21, separately (i.e., in advance) from the series of procedures to change the difficulty levels in the information processor 1 illustrated in FIG. 20. At this time, the user who answers the questions to derive the regression formula 23a and the user who answers the questions in the series of procedures illustrated in FIG. 20 may be the same as or different from each other. It is to be noted that the information processor 1 may perform the series of procedures for deriving the regression formula 23a illustrated in FIG. 21 to be mixed into the series of procedures of steps S121 to S123 illustrated in FIG. 20.

In the present modification example, a regression table 23a is used. Also in such a case, it is possible to derive the cognitive capacity regardless of whether or not there is correct reaction time 25.

### Modification Example C

In the foregoing embodiment, for example, as illustrated in FIG. 22, an information processing program 21b and a regression formula 23b may be stored in the storage unit 20, instead of the information processing program 21 and the regression formula 23. The regression formula 23b is, for example, the regression formula illustrated in FIG. 11.

The signal processing unit 30 executes the information processing program 21b stored in the storage unit 20. The functions of the signal processing unit 30 are implemented, for example, by execution of the information processing program 21b by the signal processing unit 30. For example, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. When acquiring an answer corresponding to the question data 22 from the input reception unit 10, for example, the signal processing unit 30 derives the reaction time 25 on the basis of an input timing of the acquired answer. For example, when provision of the predetermined number N of questions is completed, the signal processing unit 30 calculates the dispersion tv of the reaction times 25. On the basis of the calculated dispersion tv and the regression formula 23b read from the storage unit 20, for example, the signal processing unit 30 derives a cognitive capacity. On the basis of the derived cognitive capacity, for example, the signal processing unit 30 determines the difficulty level of questions to be provided subsequently. On the basis of a table included in the difficulty level 24 read from the storage unit 20, for example, the signal processing unit 30 determines the difficulty level of the questions to be provided subsequently. For example, the signal processing unit 30 writes the determined difficulty levels into the setting data of the storage unit 20 to thereby set the difficulty level of the questions to be provided subsequently.

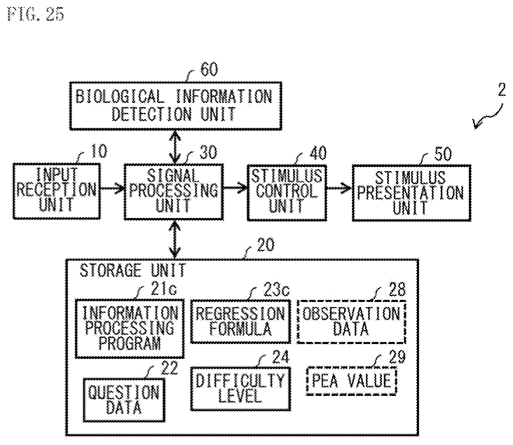

Next, description is given of operations of the information processor 1. FIG. 23 illustrates an example of a procedure to change a difficulty level in the information processor 1.

First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S141). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. When acquiring the answer from the input reception unit 10, the signal processing unit 30 calculates (acquires) the reaction time 25 for the answer corresponding to the question data (step S142).

The signal processing unit 30 executes the above-described series of processing until provision of the predetermined number N of questions is completed (step S143; N). When the provision of the predetermined number N of questions is completed (step S143; Y), the signal processing unit 30 calculates the dispersion tv of the reaction times 25 acquired thus far (step S144). The signal processing unit 30 derives a cognitive capacity using the calculated dispersion tv and the regression formula 23b read from the storage unit 20 (step S145). The signal processing unit 30 determines the difficulty level of questions to be provided subsequently using the table included in the difficulty level 24 read from the storage unit 20. The signal processing unit 30 writes the determined difficulty levels into the setting data of the storage unit 20 to thereby change the difficulty level of questions to be provided subsequently (step S146).

The signal processing unit 30 executes the above-described series of processing until the provision of the predetermined number of questions is completed (step S147; N). When the provision of the predetermined number of questions is completed (step S147; Y), the signal processing unit 30 finishes the provision.

FIG. 24 illustrates an example of a procedure to derive the regression formula 23b in the information processor 1. First, the signal processing unit 30 reads a plurality of pieces of question data of a predetermined difficulty level corresponding to the setting data included in the difficulty level 24 from among the question data 22, and sequentially outputs the plurality of pieces of read question data of the predetermined difficulty level to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S151). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. The input reception unit 10 acquires the answer corresponding to the question data from the user (step S152). The input reception unit 10 outputs the acquired answer to the signal processing unit 30. When acquiring the answer from the input reception unit 10, the signal processing unit 30 determines right or wrong of the answer corresponding to the question data using correct answer data included in the question data 22 (step S153). The signal processing unit 30 calculates (acquires) the reaction time 25 and the accuracy rate for the answer corresponding to the question data (step S154).

The signal processing unit 30 executes the above-described series of processing until provision of the predetermined number N of questions is completed (step S155; N). When the provision of the predetermined number N of questions is completed (step S155; Y), the signal processing unit 30 calculates the dispersion tv of the reaction times 25 acquired thus far and the accuracy rate R for answers acquired thus far (step S156). On the basis of the calculated dispersion tv and the accuracy rate R, the signal processing unit 30 derives the regression formula 23b, and stores the derived regression formula 23b in the storage unit 20 (step S157).

The information processor 1 may perform the series of procedures to derive the regression formula 23b illustrated in FIG. 24, separately (i.e., in advance) from the series of procedures to change the difficulty levels in the information processor 1 illustrated in FIG. 23. At this time, the user who answers the questions to derive the regression formula 23b and the user who answers the questions in the series of procedures illustrated in FIG. 23 may be the same as or different from each other. It is to be noted that the information processor 1 may perform the series of procedures for deriving the regression formula 23b illustrated in FIG. 24 to be mixed into the series of procedures of steps S141 to S143 illustrated in FIG. 23.

In the present modification example, the regression formula 23b is used. Also in such a case, it is possible to derive the cognitive capacity regardless of whether or not there is correct reaction time 25.

### Modification Example D

In the foregoing embodiment, the input reception unit 10 may accept answers from a plurality of users. In this case, the signal processing unit 30 derives the reaction times 25 for each of the users on the basis of input timings of the answers received from the respective users. The signal processing unit 30 further calculates the task difference Δtv in the dispersion of the reaction times 25 for each of the users, and derives a cognitive capacity based on the calculated task difference Δtv and the regression formula 23 read from the storage unit 20 for each of the users. On the basis of the cognitive capacity derived for each of the users, the signal processing unit 30 derives a cognitive capacity of a group when the plurality of users is viewed as a group. In such a case, it is possible to determine, for example, how much load a task causes on the group or how much margin the group has with respect to the task.

### 4. Second Embodiment

Next, description is given of an information processor 2 according to a second embodiment of the present disclosure. It is to be noted that descriptions of configurations denoted by reference numerals common to those of the foregoing embodiment are omitted as appropriate in order to avoid repetitive descriptions.

FIG. 25 illustrates an example of a schematic configuration of the information processor 2 according to the present embodiment. The information processor 2 includes the input reception unit 10, the storage unit 20, the signal processing unit 30, the stimulus control unit 40, the stimulus presentation unit 50, and a biological information detection unit 60. The biological information detection unit 60 functions as a brain wave detection unit that detects a brain wave of a user and outputs signal data of the detected brain wave to the signal processing unit 30. The signal processing unit 30 corresponds to a specific example of each of the “acquisition unit”, the “determination unit”, and the “deriving unit” of the present disclosure. The stimulus presentation unit 50 corresponds to a specific example of the “presentation unit” of the present disclosure. The biological information detection unit 60 corresponds to a specific example of a “detection unit” of the present disclosure.

In the information processor 2, the storage unit 20 stores an information processing program 21c to control the cognitive capacity of the user, and the question data 22, a regression formula 23c, and the difficulty level 24 which are to be used in the information processing program 21c. The regression formula 23c is, for example, the regression formula illustrated in FIG. 7. The regression formula 23c corresponds to a specific example of the “regression data” of the present disclosure. Further, the storage unit 20 stores observation data 28 and a peak value 29 which are obtained by processing by the information processing program 21c.

The signal processing unit 30 executes the information processing program 21c stored in the storage unit 20. The functions of the signal processing unit 30 are implemented, for example, by execution of the information processing program 21c by the signal processing unit 30. For example, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. For example, the signal processing unit 30 acquires, from the biological information detection unit 60, signal data of brain waves of the user corresponding to the plurality of pieces of question data of different difficulty levels. For example, the signal processing unit 30 extracts signal data of a waveform (α-wave) to be observed included in the acquired signal data of the brain waves. For example, the signal processing unit 30 derives the peak value 29 of power of the slow brain wave (α-wave) in the signal data of the waveform (α-wave) to be observed. For example, when the provision of the predetermined number N of questions is completed, the signal processing unit 30 calculates the task difference ΔP in the peak value 29. The task difference ΔP in the peak value 29 corresponds to a specific example of a “fluctuation in a biological signal of a user in a specific frequency band” of the present disclosure. On the basis of the calculated task difference ΔP and the regression formula 23c read from the storage unit 20, for example, the signal processing unit 30 derives a cognitive capacity. On the basis of the derived cognitive capacity, for example, the signal processing unit 30 determines the difficulty level of questions to be provided subsequently. On the basis of a table included in the difficulty level 24 read from the storage unit 20, for example, the signal processing unit 30 determines the difficulty level of the questions to be provided subsequently. For example, the signal processing unit 30 writes the determined difficulty levels into the setting data of the storage unit 20 to thereby set the difficulty level of the questions to be provided subsequently.

### [Operations]

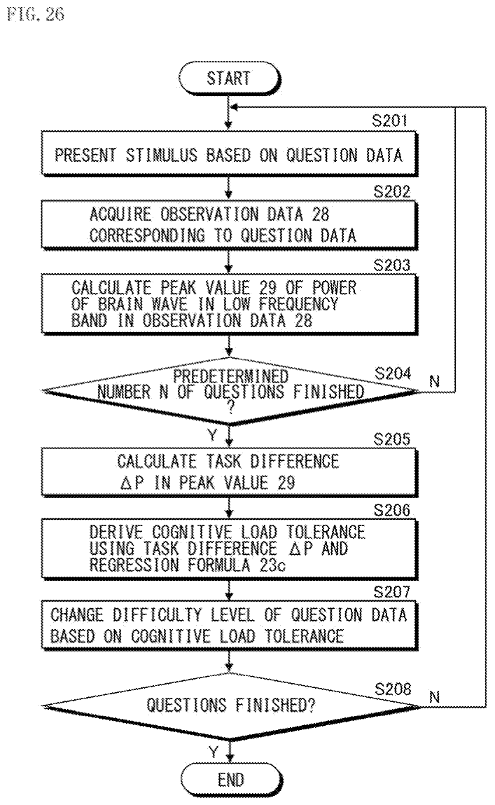

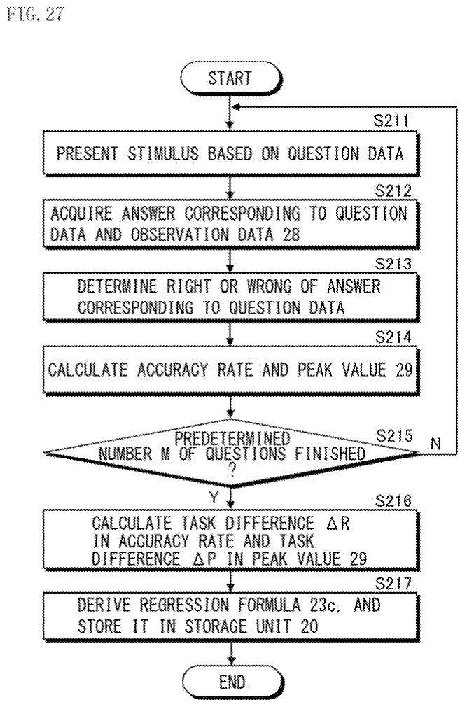

Next, description is given of operations of the information processor 2. FIG. 26 illustrates an example of a procedure to change a difficulty level in the information processor 2.

First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S201). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. During this time, the biological information detection unit 60 detects brain waves of the user, and outputs signal data of the detected brain waves to the signal processing unit 30.

The signal processing unit 30 acquires, from the biological information detection unit 60, signal data (observation data 28) of the brain waves of the user corresponding to the plurality of pieces of question data of different difficulty levels (step S202). The signal processing unit 30 extracts signal data of the waveform (α-wave) to be observed included in the acquired signal data of the brain waves. The signal processing unit 30 calculates the peak value 29 of the power of the slow brain wave (α-wave) in the signal data of the waveform (α-wave) to be observed (step S203).

The signal processing unit 30 executes the above-described series of processing until provision of the predetermined number N of questions is completed (step S204; N). When the provision of the predetermined number N of questions is completed (step S204; Y), the signal processing unit 30 calculates the task difference ΔP in the peak values 29 calculated thus far (step S205). The signal processing unit 30 derives a cognitive capacity using the calculated task difference ΔP and the regression formula 23c read from the storage unit 20 (step S206). The signal processing unit 30 determines the difficulty level of questions to be provided subsequently using the table included in the difficulty level 24 read from the storage unit 20. The signal processing unit 30 writes the determined difficulty levels into the setting data of the storage unit 20 to thereby change the difficulty level of questions to be provided subsequently (step S207).

The signal processing unit 30 executes the above-described series of processing until the provision of the predetermined number of questions is completed (step S208; N). When the provision of the predetermined number of questions is completed (step S208; Y), the signal processing unit 30 finishes the provision.

FIG. 27 illustrates an example of a procedure to derive the regression formula 23c in the information processor 2. First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S211). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. During this time, the biological information detection unit 60 detects brain waves of the user, and outputs signal data of the detected brain waves to the signal processing unit 30.

The signal processing unit 30 acquires, from the biological information detection unit 60, signal data (observation data 28) of the brain waves of the user corresponding to the plurality of pieces of question data of different difficulty levels (step S212). The signal processing unit 30 further acquires answers corresponding to the plurality of pieces of question data of different difficulty levels (step S212). The signal processing unit 30 determines right or wrong of the answer corresponding to the question data, acquired from the input reception unit 10, using correct answer data included in the question data 22 (step S213). The signal processing unit 30 calculates (acquires) an accuracy rate for the answer corresponding to the question data (step S214). The signal processing unit 30 further extracts signal data of the waveform (α-wave) to be observed included in the acquired signal data (observation data 28) of the brain waves. The signal processing unit 30 calculates the peak value 29 of the power of the slow brain wave (α-wave) in the signal data of the waveform (α-wave) to be observed (step S214).

The signal processing unit 30 executes the above-described series of processing until provision of the predetermined number N of questions is completed (step S215; N). When the provision of the predetermined number N of questions is completed (step S215; Y), the signal processing unit 30 calculates the task difference ΔR in the accuracy rate for answers acquired thus far and the task difference ΔP in the peak values 29 calculated thus far (step S216). On the basis of the calculated task differences ΔR and ΔP, the signal processing unit 30 derives the regression formula 23c, and stores the derived regression formula 23c in the storage unit 20 (step S217).

The information processor 2 may perform the series of procedures to derive the regression formula 23c illustrated in FIG. 27, separately (i.e., in advance) from the series of procedures to change the difficulty levels in the information processor 2 illustrated in FIG. 26. At this time, the user who answers the questions to derive the regression formula 23c and the user who answers the questions in the series of procedures illustrated in FIG. 26 may be the same as or different from each other. It is to be noted that the information processor 2 may perform the series of procedures for deriving the regression formula 23c illustrated in FIG. 27 to be mixed into the series of procedures of steps S201 to S204 illustrated in FIG. 26.

### [Effects]

Next, description is given of effects of the information processor 2.

In the information processor 2 and the information processing program 21c according to the present embodiment, a plurality of pieces of question data to be presented to the user is determined on the basis of a fluctuation (peak value 29) in the biological signal of the user in a specific frequency band with respect to the question data. Here, the present discloser has experimentally obtained knowledge that the fluctuation (peak value 29) in the biological signal of the user in the specific frequency band varies depending on tasks. It is therefore possible to determine the question data to be presented to the user on the basis of the fluctuation (peak value 29) in the biological signal of the user in the specific frequency band. Thus, it is possible to derive the cognitive capacity regardless of whether or not there is reaction time.

In the information processor 2 and the information processing program 21c according to the present embodiment, the cognitive capacity of the user is derived on the basis of the fluctuation (peak value 29) in the biological signal of the user in the specific frequency band with respect to the question data. This makes it possible, on the basis of the derived cognitive capacity, to determine the difficulty level of question data to be presented to the user and to determine subsequent question data. Thus, it is possible to derive the cognitive capacity regardless of whether or not there is reaction time.

In the information processor 2 and the information processing program 21c according to the present embodiment, the cognitive capacity is derived on the basis of the task difference ΔP in the fluctuation (peak value 29) in the biological signal of the user in the specific frequency band and the regression formula 23c for the task difference ΔP. This makes it possible to derive the cognitive capacity regardless of whether or not there is reaction time.

In the information processor 2 and the information processing program 21c according to the present embodiment, the difficulty levels of the plurality of pieces of question data to be provided subsequently are determined by using the table in which the difficulty levels corresponding to the cognitive capacity are set. This makes it possible to set the difficulty levels of the plurality of pieces of question data to be provided subsequently, for example, to bring the cognitive capacity of the user closer to a predetermined standard.

In the information processor 2 according to the present embodiment, the biological signal of the user is detected by the biological information detection unit 60, and is outputted to the signal processing unit 30. This makes it possible to derive the cognitive capacity on the basis of the task difference ΔP in the fluctuation (peak value 29) in the biological signal of the user in the specific frequency band and the regression formula 23c for the task difference ΔP. As a result, it is possible to derive the cognitive capacity without acquiring the reaction time 25. That is, it is possible to derive the cognitive capacity regardless of whether or not there is reaction time.

The information processor 2 according to the present embodiment is provided with the stimulus presentation unit 50 that presents the plurality of pieces of question data of different difficulty levels. This makes it possible to control presentation timings of the respective pieces of question data, thus making it possible to accurately derive the fluctuation (peak value 29) in the biological signal of the user in the specific frequency band corresponding to the plurality of pieces of question data of different difficulty levels. As a result, it is possible to derive the cognitive capacity without acquiring the reaction time 25. That is, it is possible to derive the cognitive capacity regardless of whether or not there is reaction time.

It is to be noted that, in the present embodiment, similarly to the foregoing modification examples of the first embodiment, the regression formula 23c may be a regression formula defining a relationship between the task difference ΔP and the accuracy rate R at the time of solving the high difficulty level questions, or may be a regression formula defining a relationship between the peak value of the power of the slow brain wave (α-wave) and the task difference ΔR. In addition, in the present modification example, similarly to the foregoing modification examples of the first embodiment, the regression formula 23c may be a regression formula defining a relationship between the peak value of the power of the slow brain wave (α-wave) and the accuracy rate R at the time of solving the high difficulty level questions.

### 5. Modification Example of Second Embodiment

In the foregoing second embodiment, for example, a regression table similar to the regression table 26 illustrated in FIG. 18 may be stored in the storage unit 20, instead of the regression formula 23c. In the regression table according to the present modification example, a correspondence relationship between the task difference ΔP [(mV²/Hz)²/Hz] and the task difference ΔR [%] is set using a table. The task difference ΔP [(mV²/Hz)²/Hz] is a task difference in the peak value of the power of the slow brain wave (α-wave) of the user between the time of solving the high difficulty level questions and the time of solving the low difficulty level questions. The task difference ΔR [%] is the task difference in the accuracy rate for questions between the time of solving the high difficulty level questions and the time of solving the low difficulty level questions. In the regression table according to the present modification example, for example, when the task difference ΔP [(mV²/Hz)²/Hz] is within a range of 0.4 to 0.2, the task difference ΔR [%] in the accuracy rate for questions is −3%; when the task difference ΔP [(mV²/Hz)²/Hz] is within a range of 0.2 to 0.0, the task difference ΔR [%] in the accuracy rate for questions is −8%; and when the task difference ΔP [(mV²/Hz)²/Hz] is within a range of 0 to —0.2, the task difference ΔR [%] in the accuracy rate for questions is −10%.

In the present modification example, a regression table similar to the regression table 26 illustrated in FIG. 18 is used instead of the regression formula 23c. Also in such a case, it is possible to derive the cognitive capacity without acquiring the reaction time 25. That is, it is possible to derive the cognitive capacity regardless of whether or not there is correct reaction time 25

In addition, in the foregoing second embodiment and modification example thereof, the biological information detection unit 60 may detect brain waves of a plurality of users. In this case, the signal processing unit 30 extracts signal data of the waveform (α-wave) to be observed included in the signal data of the brain wave obtained from each of the users. The signal processing unit 30 derives the peak value 29 of the power of the slow brain wave (α-wave) in the signal data of the waveform (α-wave) to be observed for each of the users. The signal processing unit 30 further calculates the task difference ΔP in the peak value 29 for each of the users, and derives a cognitive capacity based on the calculated task difference ΔP and the regression formula 23c read from the storage unit 20 for each of the users. On the basis of the cognitive capacity derived for each of the users, the signal processing unit 30 derives a cognitive capacity of a group when the plurality of users is viewed as a group. In such a case, it is possible to determine, for example, how much load a task causes on the group or how much margin the group has with respect to the task.

### 6. Third Embodiment

### [Configuration]

Next, description is given of an information processor 3 according to a third embodiment of the present disclosure. FIG. 28 illustrates an example of a schematic configuration of the information processor 3 according to the present embodiment. The information processor 3 has a configuration in which the information processor 1 according to the foregoing first embodiment and the information processor 2 according to the foregoing second embodiment are added together. That is, the information processor 3 includes the input reception unit 10, the storage unit 20, the signal processing unit 30, the stimulus control unit 40, the stimulus presentation unit 50, and the biological information detection unit 60. The storage unit 20 stores an information processing program 21d, and the question data 22, the regression formulae 23 and 23c, and the difficulty level 24 which are to be used in the information processing program 21d. Further, the storage unit 20 stores the reaction time 25, the observation data 28 and the peak value 29 which are obtained by processing by the information processing program 21d.

### [Operations]

Next, description is given of operations of the information processor 2. FIG. 29 illustrates an example of a procedure to change a difficulty level in the information processor 3.

First, the signal processing unit 30 reads question data of a plurality of difficulty levels corresponding to setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S301). The stimulus presentation unit 50 presents to the user, for example, an image including question data, a sound speaking question data, or light corresponding to question data. The user then inputs an answer corresponding to the question data into the input reception unit 10. When acquiring the answer corresponding to the question data from the user, the input reception unit 10 outputs the acquired answer to the signal processing unit 30. During this time, the biological information detection unit 60 detects brain waves of the user, and outputs signal data of the detected brain waves to the signal processing unit 30.

The signal processing unit 30 acquires, from the biological information detection unit 60, signal data (observation data 28) of the brain waves of the user corresponding to the plurality of pieces of question data of different difficulty levels (step S302). The signal processing unit 30 extracts signal data of the waveform (α-wave) to be observed included in the acquired signal data of the brain waves. The signal processing unit 30 calculates the peak value 29 of the power of the slow brain wave (α-wave) in the signal data of the waveform (α-wave) to be observed (step S303). The signal processing unit 30 further calculates (acquires) the reaction time 25 for an answer corresponding to the question data (step S303).

The signal processing unit 30 executes the above-described series of processing until provision of the predetermined number N of questions is completed (step S304; N). When the provision of the predetermined number N of questions is completed (step S304; Y), the signal processing unit 30 calculates the task difference Δtv in the dispersion of the reaction times 25 calculated thus far and the task difference ΔP in the peak values 29 calculated thus far (step S305). The signal processing unit 30 derives a cognitive capacity using the calculated task differences Δtv and ΔP and the regression formulae 23 and 23c read from the storage unit 20 (step S306). The signal processing unit 30 determines the difficulty level of questions to be provided subsequently using the table included in the difficulty level 24 read from the storage unit 20. The signal processing unit 30 writes the determined difficulty levels into the setting data of the storage unit 20 to thereby change the difficulty level of questions to be provided subsequently (step S307).

The signal processing unit 30 executes the above-described series of processing until the provision of the predetermined number of questions is completed (step S308; N). When the provision of the predetermined number of questions is completed (step S308; Y), the signal processing unit 30 finishes the provision.

In the information processor 3 and the information processing program 21c according to the present embodiment, the difficulty levels of the plurality of pieces of question data to be presented subsequently is determined on the basis of the cognitive capacity of the user obtained on the basis of the calculated task differences Δtv and ΔP. Here, the present discloser has experimentally obtained knowledge that the dispersion of the reaction times 25 and the fluctuation (peak value 29) in the biological signal of the user in the specific frequency band vary depending on tasks. It is therefore possible to derive the cognitive capacity of the user on the basis of the dispersion of the reaction times 25 and the fluctuation (peak value 29) in the biological signal of the user in the specific frequency band, and thus to determine the difficulty levels of the plurality of pieces of question data to be provided subsequently. Thus, it is possible to derive the cognitive capacity regardless of whether or not there is correct reaction time.

### 8. Fourth Embodiment

Next, description is given of an information processor 4 according to a fourth embodiment of the present disclosure. FIG. 30 illustrates an example of a schematic configuration of the information processor 4 according to the present embodiment. The information processor 4 includes the input reception unit 10, the storage unit 20, the signal processing unit 30, the stimulus control unit 40, the stimulus presentation unit 50, and the biological information detection unit 60. The signal processing unit 30 corresponds to a specific example of each of a “characteristic value generation unit”, an “evaluation value generation unit”, the “determination unit”, and the “deriving unit” of the present disclosure. The stimulus presentation unit 50 corresponds to a specific example of the “presentation unit” of the present disclosure. The biological information detection unit 60 corresponds to a specific example of the “detection unit” of the present disclosure.

The storage unit 20 stores an information processing program 21e to control the cognitive capacity of the user, and the question data 22, a regression formula 41, the difficulty level 24, a length 44 of a division period ΔT, a length 45 of an overlap period Δd1, a length 46 of a division period ΔW, and a length 47 of an overlap period Δd2, which are to be used in the information processing program 21e. The regression formula 41 is, for example, the regression formula illustrated in FIG. 8 or 9. The regression formula 41 corresponds to a specific example of the “regression data” of the present disclosure. The information processing program 21e performs processing, for example, to generate, from acquired observation data 43, a characteristic value of a waveform to be observed for each of pieces of observation data 43, and to generate an evaluation value for a difference between the pieces of observation data 43 regarding the waveform to be observed on the basis of the characteristic value for each of the pieces of observation data 43. The division period ΔW is a value longer than the division period ΔT.

The storage unit 20 stores data inputted into the signal processing unit 30 from the input reception unit 10. The storage unit 20 stores, for example, a set value of the length of the division period ΔT inputted from the input reception unit 10, and a set value of the length of the overlap period Δd1 inputted from the input reception unit 10. It is to be noted that the set value of the division period ΔT inputted from the input reception unit 10 is included in the length 44 of the division period ΔT in the storage unit 20. In addition, the set value of the overlap period Δd1 inputted from the input reception unit 10 is included in the length 45 of the overlap period Δd1 in the storage unit 20.

The storage unit 20 further stores data (observation data 43) inputted into the signal processing unit 30 from the biological information detection unit 60. For example, as illustrated in FIG. 30, the storage unit 20 stores n pieces of observation data 43.

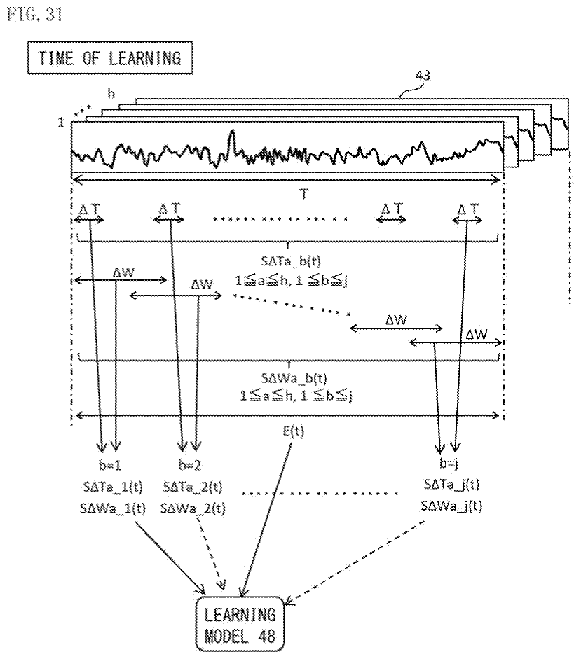

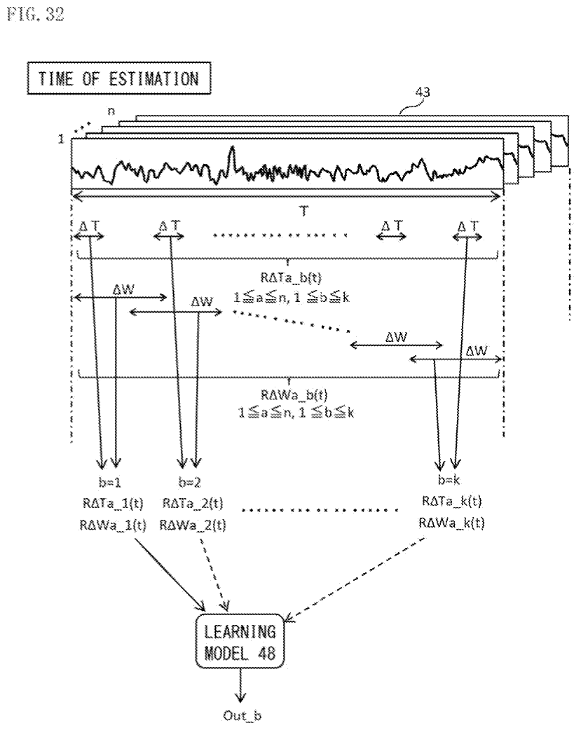

The storage unit 20 further stores a learning model 48. The learning model 48 carries out, for example, a learning procedure illustrated in FIG. 31 and an estimation procedure illustrated in FIG. 32.

Here, in each of h pieces of observation data 43 for learning, an area in a frequency band of the waveform to be observed included in a power spectrum PΔTa_b(t) (1≤a≤h, 1≤b≤j) derived for each of the division periods ΔT divided into j pieces is set as SΔTa_b(t) (characteristic value) (see FIG. 31). In addition, in each of the h pieces of observation data 43 for learning, an area in a frequency band of the waveform to be observed included in a power spectrum PΔWa_b(t) (1≤a≤h, 1≤b≤j) derived for each of the division periods ΔW divided into j pieces is set as SΔWa_b(t) (characteristic value) (see FIG. 31). In each of the h pieces of observation data 43 for learning, an emotion state within an observation period T is set as E(t). It is to be noted that the number (h pieces) of the observation data 43 for learning is equal to the number (n pieces) of the observation data 43 at the time of estimation of emotion described later. In addition, the number (k pieces) of division of the observation data 43 at the time of learning may be equal to or different from the number (j pieces) of division of the observation data 43 at the time of the estimation of emotion described later. In addition, it is assumed, in the area SΔTa_b(t) and the area SΔWa_b(t), that there are data at the same time within the observation period T.

The learning model 48 is a model in which learning is performed, including a machine learning in which, in the area SΔTa_b(t) and the area SΔWa_b(t), 2n pieces of data having b in common are set as an explanatory variable and an emotion state E(t) in a period corresponding to b, out of the emotion state E(t), is set as an objective variable (see FIG. 32). That is, the learning model 48 is a model to estimate one emotion state from the 2n pieces of data.

The signal processing unit 30 executes the information processing program 21e stored in the storage unit 20. The functions of the signal processing unit 30 are implemented, for example, by execution of the information processing program 21e by the signal processing unit 30. The signal processing unit 30 derives the power spectrum PΔTa_b(t) (1≤a≤n, 1≤b≤k) for each of the division periods ΔT divided into k pieces in each of measured n pieces of observation data 43, and derives an area RΔTa_b(t) (characteristic value) in a frequency band of the waveform to be observed included in the derived power spectrum PΔTa_b(t) (see FIG. 32). The signal processing unit 30 further derives the power spectrum PΔWa_b(t) (1≤a≤n, 1≤b≤k) for each of the division periods ΔW divided into k pieces in each of the measured n pieces of observation data 43, and derives an area RΔWa_b(t) (characteristic value) in a frequency band of the waveform to be observed included in the derived power spectrum PΔWa_b(t) (see FIG. 32).

When the 2n pieces of data having b in common are inputted, in the area RΔTa_b and the area RΔWa_b derived by the signal processing unit 30, the learning model 48 generates, on the basis of the 2n pieces of data, an emotion state Out_b, which is an evaluation value for the difference between the pieces of observation data 43 regarding the waveform to be observed, and outputs the generated emotion state Out_b to the signal processing unit 30 (see FIG. 32). The emotion state Out_b corresponds to an arousal level of the user.

Here, when actual emotion states corresponding to the area RΔTa_b and the area RΔWa_b are all the same, it is assumed that i pieces (i≤k) of the emotion states Out_b coincide with the actual emotion states. At this time, an estimation accuracy is i/k.

### [Operations]

Next, description is given of operations of the information processor 4. FIG. 33 illustrates an example of a procedure to change a difficulty level in the information processor 4.

First, the signal processing unit 30 reads question data of a plurality of difficulty levels corresponding to setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S401). The stimulus presentation unit 50 presents to the user, for example, an image including question data, a sound speaking question data, or light corresponding to question data. The user then inputs an answer corresponding to the question data into the input reception unit 10. When acquiring the answer corresponding to the question data from the user, the input reception unit 10 outputs the acquired answer to the signal processing unit 30. During this time, the biological information detection unit 60 detects brain waves of the user, and outputs signal data of the detected brain waves to the signal processing unit 30.

The signal processing unit 30 acquires, from the biological information detection unit 60, signal data (n pieces of observation data 43) of the brain waves of the user corresponding to the plurality of pieces of question data of different difficulty levels (step S402). The signal processing unit 30 derives the power spectrum PΔTa_b(t) (1≤a≤n, 1≤b≤k) for each of the division periods ΔT divided into k pieces in each of the acquired n pieces of observation data 43, and derives the area RΔTa_b(t) (characteristic value) in the frequency band of the waveform to be observed included in the derived power spectrum PΔTa_b(t). The signal processing unit 30 further derives the power spectrum PΔWa_b(t) (1≤a≤n, 1≤b≤k) for each of the division periods ΔW divided into k pieces in each of the n pieces of observation data 43, and derives the area RΔWa_b(t) (characteristic value) in the frequency band of the waveform to be observed included in the derived power spectrum PΔWa_b(t).

The signal processing unit 30 inputs 2n pieces of data having b in common into the learning model 48 in the areas RΔTa_b and the area RΔWa_b which are derived. The learning model 48 then outputs the emotion state Out_b (evaluation value) corresponding to the 2n pieces of data to the signal processing unit 30. That is, the signal processing unit 30 derives the emotion state Out_b (arousal level 42) using the n pieces of observation data 43 and the learning model 48 (step S403).

The signal processing unit 30 executes the above-described series of processing until provision of the predetermined number N of questions is completed (step S404; N). When the provision of the predetermined number N of questions is completed (step S404; Y), the signal processing unit 30 calculates the task difference Δk in the arousal levels 42 calculated thus far (step S405). The signal processing unit 30 derives a cognitive capacity on the basis of the calculated task difference Δk and the regression formula 41 read from the storage unit 20 (step S206). The signal processing unit 30 determines the difficulty level of questions to be provided subsequently on the basis of the derived cognition capacity, for example. The signal processing unit 30 determines the difficulty level of questions to be provided subsequently on the basis of the table included in the difficulty level 24 read from the storage unit 20. The signal processing unit 30 writes the determined difficulty levels into the setting data of the storage unit 20 to thereby change the difficulty level of questions to be provided subsequently (step S407).

The signal processing unit 30 executes the above-described series of processing until the provision of the predetermined number of questions is completed (step S408; N). When the provision of the predetermined number of questions is completed (step S408; Y), the signal processing unit 30 finishes the provision.

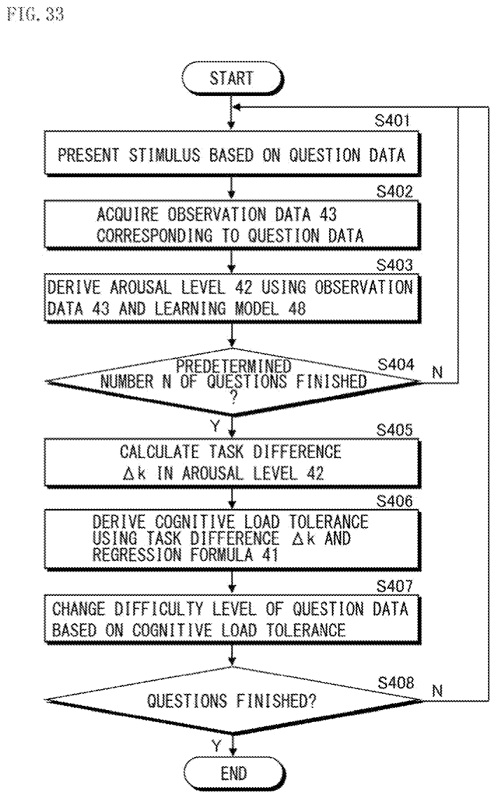

FIG. 34 illustrates an example of a procedure to derive the regression formula 41 in the information processor 4. First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S411). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. During this time, the biological information detection unit 60 detects brain waves of the user, and outputs signal data (n pieces of observation data 43) of the detected brain waves to the signal processing unit 30.

The signal processing unit 30 acquires, from the biological information detection unit 60, signal data (n pieces of observation data 43) of the brain waves of the user corresponding to the plurality of pieces of question data of different difficulty levels (step S412). The signal processing unit 30 further acquires answers corresponding to the plurality of pieces of question data of different difficulty levels (step S412). The signal processing unit 30 determines right or wrong of the answer corresponding to the question data, acquired from the input reception unit 10, using correct answer data included in the question data 22 (step S413). The signal processing unit 30 calculates (acquires) an accuracy rate for the answer corresponding to the question data (step S414). The signal processing unit 30 further derives the emotion state Out_b (arousal levels 42) using the acquired signal data (n pieces of observation data 43) of the brain waves and the learning model 48 (step S414).

The signal processing unit 30 executes the above-described series of processing until provision of the predetermined number N of questions is completed (step S415; N). When the provision of the predetermined number N of questions is completed (step S415; Y), the signal processing unit 30 calculates the task difference ΔR in the accuracy rate for answers acquired thus far and the task difference Δk in the arousal levels 42 calculated thus far (step S416). On the basis of the calculated task differences ΔR and Δk, the signal processing unit 30 derives the regression formula 41, and stores the derived regression formula 41 in the storage unit 20 (step S417).

### [Effects]

Next, description is given of effects of the information processor 4.

In the information processor 4 and the information processing program 21e according to the present embodiment, the characteristic values (area RΔTa_b(t) and area RΔWa_b(t)) for each of the pieces of observation data 43 are derived from each of the pieces of observation data 43 obtained by biological observation of the user in a predetermined period of time, and an evaluation value (emotion state Out_b) for a difference between the pieces of observation data regarding the waveform to be observed is generated as the arousal level 42 on the basis of the derived characteristic value for each of the pieces of observation data 43. Then, a task for the user is determined on the basis of the generated evaluation value (arousal level 42). Here, the present discloser has experimentally obtained knowledge that the above-described evaluation value (arousal level 42) varies depending on tasks. It is therefore possible to determine the question data to be presented to the user on the basis of the above-described evaluation value (arousal levels 42). Thus, it is possible to derive the cognitive capacity regardless of whether or not there is reaction time.

In the information processor 4 and the information processing program 21e according to the present embodiment, the cognitive capacity of the user is derived on the basis of the above-described evaluation value (arousal level 42). This makes it possible, on the basis of the derived cognitive capacity, to determine the difficulty level of question data to be presented to the user and to determine subsequent question data. Thus, it is possible to derive the cognitive capacity regardless of whether or not there is reaction time.

In the information processor 4 and the information processing program 21e according to the present embodiment, the cognitive capacity is derived on the basis of the task difference Δk in the evaluation value (arousal level 42) and the regression formula 41 for the task difference Δk in the evaluation value (arousal level 42). This makes it possible to derive the cognitive capacity regardless of whether or not there is reaction time.

In the information processor 4 and the information processing program 21e according to the present embodiment, the difficulty levels of the plurality of pieces of question data to be provided subsequently are determined by using the table in which the difficulty levels corresponding to the cognitive capacity are set. This makes it possible to set the difficulty levels of the plurality of pieces of question data to be provided subsequently, for example, to bring the cognitive capacity of the user closer to a predetermined standard.

The information processor 4 according to the present embodiment is provided with the stimulus presentation unit 50 that presents the plurality of pieces of question data of different difficulty levels. This makes it possible to control presentation timings of respective pieces of question data, thus making it possible to accurately derive the evaluation value (arousal level 42). As a result, it is possible to accurately derive the cognitive capacity regardless of whether or not there is reaction time.

### 9. Modification Examples of Fourth Embodiment

### Modification Example E

In the foregoing fourth embodiment, for example, as illustrated in FIG. 35, an information processing program 21f and a regression formula 41a may be stored in the storage unit 20, instead of the information processing program 21e and the regression formula 41.

The information processing program 21f performs processing, for example, to generate, from acquired observation data 43, a characteristic value of a waveform to be observed for each of the pieces of observation data 43, and to generate an evaluation value for a difference between the pieces of observation data 43 regarding the waveform to be observed, on the basis of the characteristic value for each of the pieces of observation data 43. The regression formula 41 an is, for example, the regression formula illustrated in FIG. 12.

The signal processing unit 30 executes the information processing program 21f stored in the storage unit 20. The functions of the signal processing unit 30 are implemented, for example, by execution of the information processing program 21f by the signal processing unit 30.

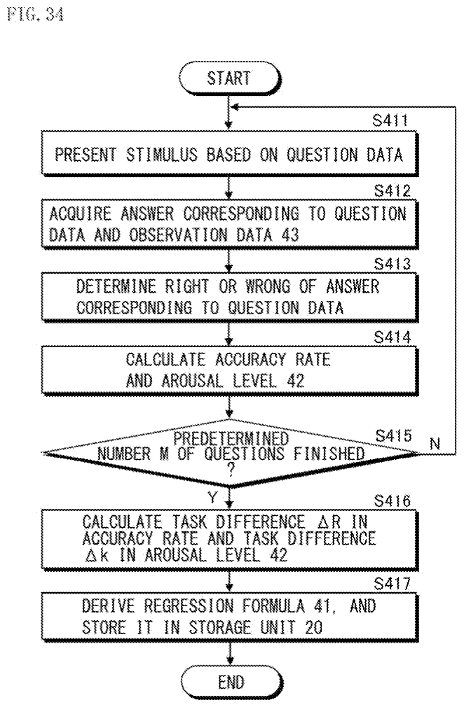

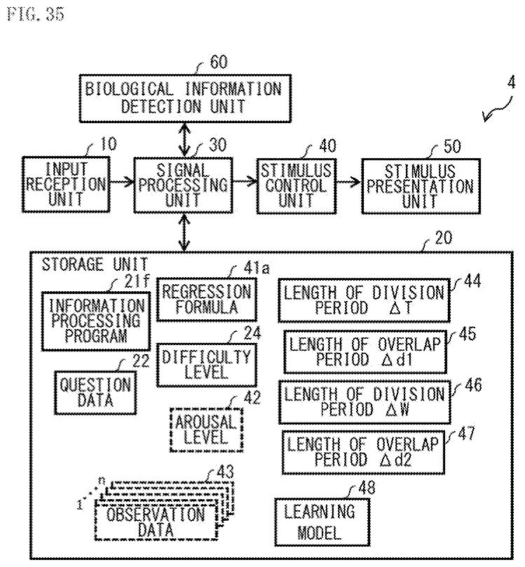

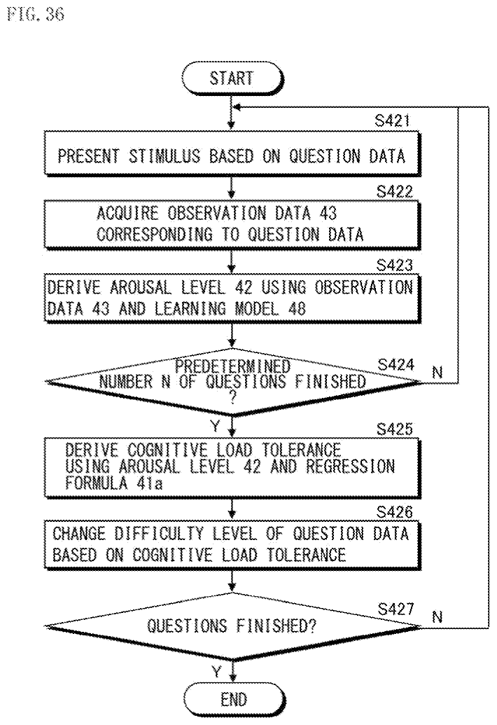

Next, description is given of operations of the information processor 4 in the present modification example. FIG. 36 illustrates an example of a procedure to change a difficulty level in the information processor 4.

First, the signal processing unit 30 reads a plurality of pieces of question data of a predetermined difficulty level corresponding to the setting data included in the difficulty level 24 from among the question data 22, and sequentially outputs the plurality of pieces of read question data of the predetermined difficulty level to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S421). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. During this time, the biological information detection unit 60 detects brain waves of the user, and outputs signal data of the detected brain waves to the signal processing unit 30.

The signal processing unit 30 acquires, from the biological information detection unit 60, signal data (n pieces of observation data 43) of the brain waves of the user corresponding to the plurality of pieces of question data of the predetermined difficulty level (step S422). The signal processing unit 30 derives the power spectrum PΔTa_b(t) (1≤a≤n, 1≤b≤k) for each of the division periods ΔT divided into k pieces in each of the acquired n pieces of observation data 43, and derives the area RΔTa_b(t) (characteristic value) in the frequency band of the waveform to be observed included in the derived power spectrum PΔTa_b(t). The signal processing unit 30 further derives the power spectrum PΔWa_b(t) (1≤a≤n, 1≤b≤k) for each of the division periods ΔW divided into k pieces in each of the n pieces of observation data 43, and derives the area RΔWa_b(t) (characteristic value) in the frequency band of the waveform to be observed included in the derived power spectrum PΔWa_b(t).

The signal processing unit 30 inputs 2n pieces of data having b in common into the learning model 48 in the areas RΔTa_b and the area RΔWa_b which are derived. The learning model 48 then outputs the emotion state Out_b (evaluation value) corresponding to the 2n pieces of data to the signal processing unit 30. That is, the signal processing unit 30 derives the emotion state Out_b (arousal level 42) using the n pieces of observation data 43 and the learning model 48 (step S423).

The signal processing unit 30 executes the above-described series of processing until provision of the predetermined number N of questions is completed (step S424; N). When the provision of the predetermined number N of questions is completed (step S424; Y), the signal processing unit 30 derives a cognitive capacity on the basis of the arousal levels 42 calculated thus far and the regression formula 41a read from the storage unit 20 (step S425). The signal processing unit 30 determines the difficulty level of questions to be provided subsequently on the basis of the derived cognition capacity, for example. The signal processing unit 30 determines the difficulty level of questions to be provided subsequently on the basis of the table included in the difficulty level 24 read from the storage unit 20. The signal processing unit 30 writes the determined difficulty levels into the setting data of the storage unit 20 to thereby change the difficulty level of questions to be provided subsequently (step S426).

The signal processing unit 30 executes the above-described series of processing until the provision of the predetermined number of questions is completed (step S427; N). When the provision of the predetermined number of questions is completed (step S427; Y), the signal processing unit 30 finishes the provision.

FIG. 37 illustrates an example of a procedure to derive the regression formula 41a in the information processor 4 in the present modification example. First, the signal processing unit 30 reads question data of a predetermined difficulty level corresponding to the setting data included in the difficulty level 24 from among the question data 22, and sequentially outputs a plurality of pieces of read question data of the predetermined difficulty level to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S431). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. During this time, the biological information detection unit 60 detects brain waves of the user, and outputs signal data (n pieces of observation data 43) of the detected brain waves to the signal processing unit 30.

The signal processing unit 30 acquires, from the biological information detection unit 60, signal data (n pieces of observation data 43) of the brain waves of the user corresponding to the plurality of pieces of question data of the predetermined difficulty level (step S432). The signal processing unit 30 further acquires answers corresponding to the plurality of pieces of question data of the predetermined difficulty level (step S432). The signal processing unit 30 determines right or wrong of the answer corresponding to the question data, acquired from the input reception unit 10, using correct answer data included in the question data 22 (step S433). The signal processing unit 30 calculates (acquires) an accuracy rate for the answer corresponding to the question data (step S434). The signal processing unit 30 further derives the emotion state Out_b (arousal levels 42) using the acquired signal data (n pieces of observation data 43) of the brain waves and the learning model 48 (step S434).

The signal processing unit 30 executes the above-described series of processing until provision of the predetermined number N of questions is completed (step S435; N). When the provision of the predetermined number N of questions is completed (step S435; Y), the signal processing unit 30 calculates the accuracy rate R for answers acquired thus far and the arousal levels 42 calculated thus far (step S436). On the basis of the calculated accuracy rate R and arousal levels 42, the signal processing unit 30 derives the regression formula 41a, and stores the derived regression formula 41a in the storage unit 20 (step S437).

In the present modification example, the regression formula 41a is used. Also in such a case, it is possible to derive the cognitive capacity regardless of whether or not there is correct reaction time 25.

It is to be noted that, in the present modification example, the regression formula 41a may be a regression formula defining a relationship between the arousal level k and the task difference ΔR in the accuracy rate, or may be a regression formula defining a relationship between the task difference Δk in the arousal level and the accuracy rate R.

### Modification Example F

In the foregoing fourth embodiment and modification example thereof, the biological information detection unit 60 may detect brain waves of a plurality of users. In this case, on the basis of signal data of the brain waves obtained from the respective users, the signal processing unit 30 derives the emotion state Out_b (arousal level 42) for each of the users, and calculates the task difference Δk in the derived emotion state Out_b (arousal level 42) for each of the users. On the basis of the calculated task difference Δk and the regression formula 41 read from the storage unit 20, the signal processing unit 30 derives a cognitive capacity for each of the users. On the basis of the cognitive capacity derived for each of the users, the signal processing unit 30 derives a cognitive capacity of a group when the plurality of users is viewed as a group. In such a case, it is possible to determine, for example, how much load a task causes on the group or how much margin the group has with respect to the task.

### 10. Modification Example of First Embodiment

### Modification Example G

In the foregoing first embodiment, for example, as illustrated in FIG. 38, an information processing program 21g and a regression formula 23g may be stored in the storage unit 20, instead of the information processing program 21 and the regression formula 23. The regression formula 23g is, for example, the regression formula illustrated in FIG. 13.

The signal processing unit 30 executes the information processing program 21g stored in the storage unit 20. The functions of the signal processing unit 30 are implemented, for example, by execution of the information processing program 21g by the signal processing unit 30. For example, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. When acquiring an answer corresponding to the question data 22 from the input reception unit 10, for example, the signal processing unit 30 derives the reaction time 25 on the basis of an input timing of the acquired answer. For example, when provision of the predetermined number N of questions is completed, the signal processing unit 30 calculates the task difference Δtv in the dispersion tv of the reaction times 25. On the basis of the calculated task difference Δtv and the regression formula 23g read from the storage unit 20, for example, the signal processing unit 30 derives a cognitive capacity. On the basis of the derived cognitive capacity, for example, the signal processing unit 30 determines the difficulty level of questions to be provided subsequently. On the basis of a table included in the difficulty level 24 read from the storage unit 20, for example, the signal processing unit 30 determines the difficulty level of the questions to be provided subsequently. For example, the signal processing unit 30 writes the determined difficulty levels into the setting data of the storage unit 20 to thereby set the difficulty level of the questions to be provided subsequently.

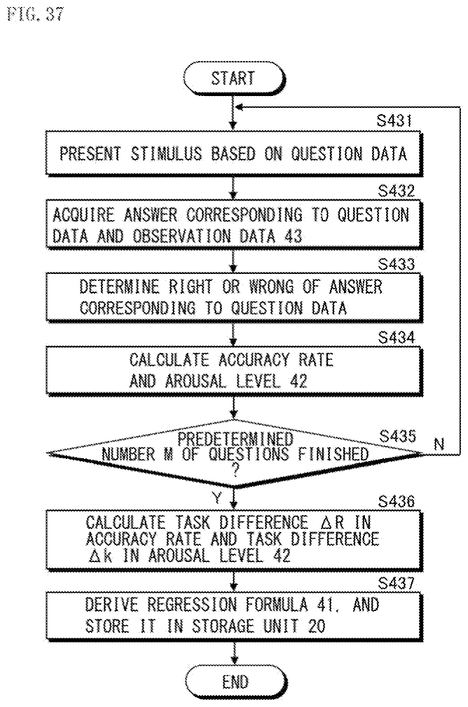

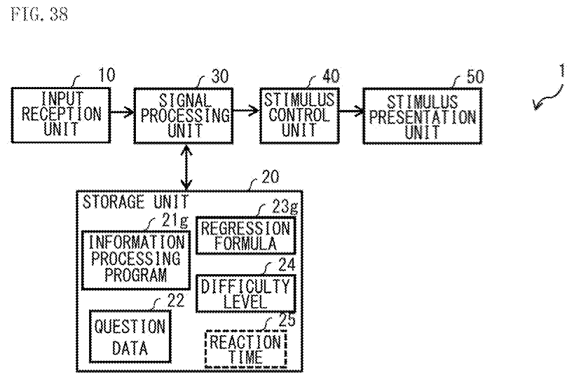

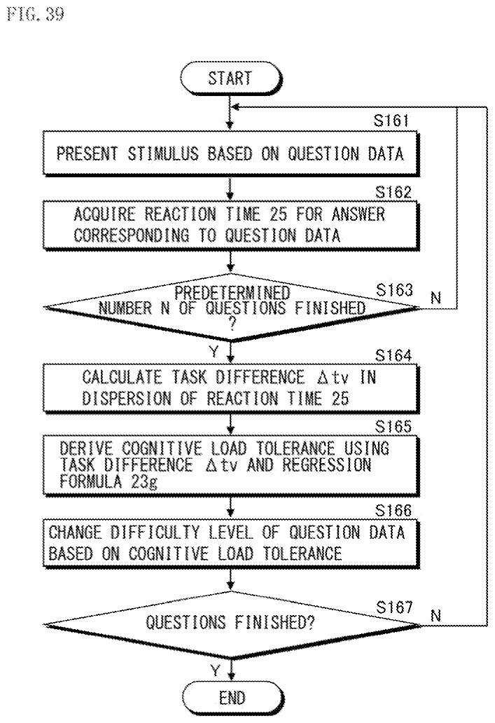

Next, description is given of operations of the information processor 1. FIG. 39 illustrates an example of a procedure to change a difficulty level in the information processor 1.

First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S161). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. When acquiring the answer corresponding to the question data from the user, the input reception unit 10 outputs the acquired answer to the signal processing unit 30. When acquiring the answer from the input reception unit 10, the signal processing unit 30 calculates (acquires) the reaction time 25 for the answer corresponding to the question data (step S162).

The signal processing unit 30 executes the above-described series of processing until provision of the predetermined number N of questions is completed (step S163; N). When the provision of the predetermined number N of questions is completed (step S163; Y), the signal processing unit 30 calculates the task difference Δtv in the dispersion tv of the reaction times 25 acquired thus far (step S164). The signal processing unit 30 derives a cognitive capacity using the calculated task difference Δtv and the regression formula 23g read from the storage unit 20 (step S165). The signal processing unit 30 determines the difficulty level of questions to be provided subsequently using the table included in the difficulty level 24 read from the storage unit 20. The signal processing unit 30 writes the determined difficulty levels into the setting data of the storage unit 20 to thereby change the difficulty level of questions to be provided subsequently (step S166).

The signal processing unit 30 executes the above-described series of processing until the provision of the predetermined number of questions is completed (step S167; N). When the provision of the predetermined number of questions is completed (step S167; Y), the signal processing unit 30 finishes the provision.

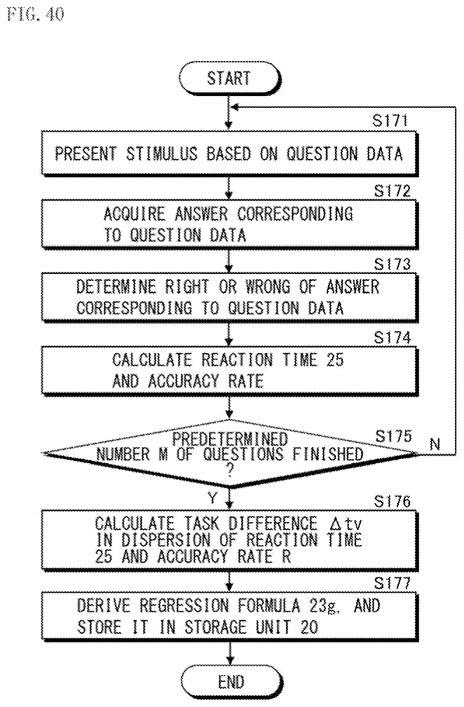

FIG. 40 illustrates an example of a procedure to derive the regression formula 23g in the information processor 1. First, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. On the basis of the question data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on question data of a predetermined difficulty level (step S171). The stimulus presentation unit 50 presents to the user, for example, an image including question data, a sound speaking question data, or light corresponding to question data. It is to be noted that the stimulus presentation unit 50 may present to the user, for example, a taste, a tactile feeling, an odor, or the like corresponding to question data. The user then inputs an answer corresponding to the question data into the input reception unit 10. The input reception unit 10 acquires the answer corresponding to the question data from the user (step S172). The input reception unit 10 outputs the acquired answer to the signal processing unit 30. When acquiring the answer from the input reception unit 10, the signal processing unit 30 determines right or wrong of the answer corresponding to the question data using correct answer data included in the question data 22 (step S173). The signal processing unit 30 calculates (acquires) the reaction time 25 and the accuracy rate for the answer corresponding to the question data (step S174).

The signal processing unit 30 executes the above-described series of processing until provision of the predetermined number N of questions is completed (step S175; N). When the provision of the predetermined number N of questions is completed (step S175; Y), the signal processing unit 30 calculates the task difference Δtv in the dispersion tv of the reaction times 25 acquired thus far and the accuracy rate R for answers acquired thus far (step S176). On the basis of the calculated task difference Δtv and the accuracy rate R, the signal processing unit 30 derives the regression formula 23g, and stores the derived regression formula 23g in the storage unit 20 (step S177).

The information processor 1 may perform the series of procedures to derive the regression formula 23g illustrated in FIG. 40, separately (i.e., in advance) from the series of procedures to change the difficulty levels in the information processor 1 illustrated in FIG. 39. At this time, the user who answers the questions to derive the regression formula 23g and the user who answers the questions in the series of procedures illustrated in FIG. 39 may be the same as or different from each other. It is to be noted that the information processor 1 may perform the series of procedures for deriving the regression formula 23g illustrated in FIG. 40 to be mixed into the series of procedures of steps S161 to S163 illustrated in FIG. 39.

In the present modification example, the regression formula 23g is used. Also in such a case, it is possible to derive the cognitive capacity regardless of whether or not there is correct reaction time 25.

### 11. Concerning Biological Information Enabling Control of Cognitive Capacity of Present Disclosure

In each of the foregoing embodiments and modification examples thereof, the reaction time and the brain wave are mentioned as information enabling control of the cognitive capacity of the present disclosure. However, possible examples of the information enabling control of the cognitive capacity of the present disclosure may include, a pulse wave, an electrocardiogram, a blood flow, emotional sweating, and the like, in addition to the reaction time and the brain wave. The pulse wave, the electrocardiogram, the blood flow, and the emotional sweating are measurable by placing a sensor (hereinafter, referred to as a “sensor S”) on a finger, an ear, a head, an arm, a chest, and the like, for example. Therefore, a large-scale sensor involving a headset or the like to be used when measuring a brain wave is not necessary as the sensor S.

The sensor S may be mounted in a head-mounted display (HMD) 200, for example, as illustrated in FIG. 41. In the head-mounted display 200, for example, a detection electrode 203 of the sensor S may be provided on an inner surface or the like, of each of a pad part 201 and a band part 202.

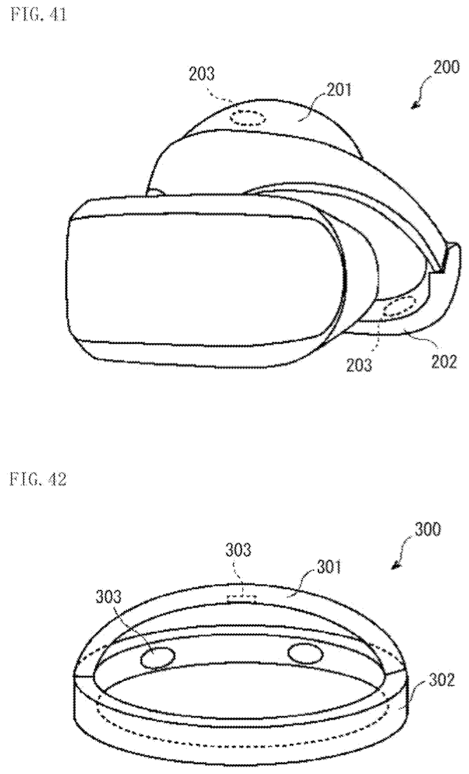

In addition, the sensor S may be mounted, for example, in a head band 300 as illustrated in FIG. 42. In the head band 300, for example, a detection electrode 303 of the sensor S may be provided on an inner surface or the like of each of band parts 301 and 302 to be in contact with the head.

In addition, the sensor S may be mounted, for example, in a headphone 400 as illustrated in FIG. 43. In the headphone 400, for example, a detection electrode 403 of the sensor S may be provided on an inner surface of a band part 401, an ear pad 402, or the like to be in contact with the head.

In addition, the sensor S may be mounted, for example, in an earphone 500 as illustrated in FIG. 44. In the earphone 500, for example, a detection electrode 502 of the sensor S may be provided in an ear piece 501 to be inserted into the ear.

In addition, the sensor S may be mounted, for example, in a watch 600 as illustrated in FIG. 45. In the watch 600, for example, a detection electrode 604 of the sensor S may be provided on an inner surface of a display part 601 that displays time or the like, an inner surface of a band part 602 (e.g., an inner surface of a buckle part 603), or the like.

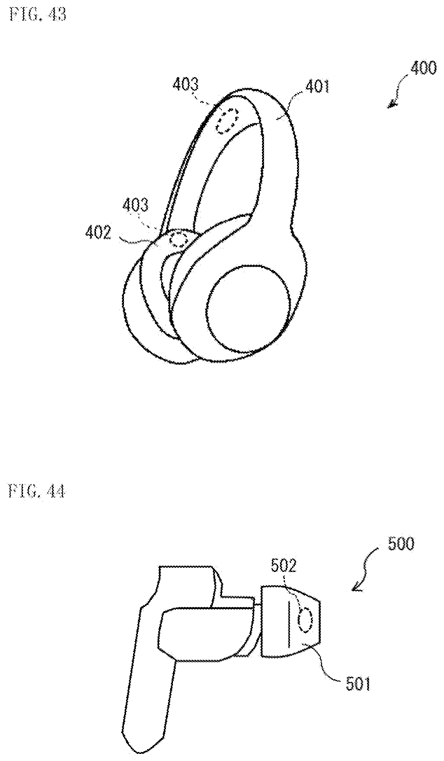

In addition, the sensor S may be mounted, for example, in glasses 700 as illustrated in FIG. 46. In the glasses 700, for example, a detection electrode 702 of the sensor S may be provided on an inner surface of a temple 701 or the like.

In addition, the sensor S may also be mounted, for example, in a glove, a ring, a pencil, a pen, a controller of a game machine, or the like.

### (Pulse Wave, Electrocardiogram, and Blood Flow)

It is possible to control the cognitive capacity of the present disclosure by using, for example, feature amounts as given below, which are obtained on the basis of electric signals of a pulse wave, an electrocardiogram, and a blood flow obtained by the sensor S.

Heart rate per 1 s

Average value of heart rates per 1 s within a predetermined period (window)

rmssd (root mean square successive difference): root mean square of successive heartbeat intervals

pnn50 (percentage of adjacent normal-to-normal intervals): percentage of the number of successive heartbeat intervals exceeding 50 ms

LF: area of PSD of heartbeat intervals between 0.04 Hz and 0.15 Hz

HF: area of PSD of heartbeat intervals between 0.15 Hz and 0.4 Hz

LF/(LF+HF)

HF/(LF+HF)

LF/HF

Heart rate entropy

SD1: standard deviation of Poincare plot (scatter diagram with an x-axis for t-th heartbeat interval and a y-axis for t+1-th heartbeat interval) in a direction of an axis of y=x

SD2: standard deviation of Poincare plot in a direction of an axis perpendicular to y=x

SD1/SD2

SDRR (standard deviation of RR interval): standard deviation of heartbeat interval

### (Emotional Sweating)

It is possible to control the cognitive capacity of the present disclosure by using, for example, feature amounts as given below, which are obtained on the basis of electric signals (EDA: electrodermal activity) of emotional sweating obtained by the sensor S

Number of SCRs (skin conductance response) occurring per minute

Amplitude of SCR

Value of SCL (skin conductance level)

Rate of change of SCL

For example, it is possible to separate SCR and SCL from EDA by using a method described in the following literature:
Benedek, M., & Kaernbach, C. (2010). A continuous measure of phasic electrodermal activity. Journal of neuroscience methods, 190(1), 80-91.

It is to be noted that, in the control of the cognitive capacity of the present disclosure, a single modal (one physiological index) may be used, or a combination of a plurality of modals (a plurality of physiological indexes) may be used.

### 12. Fifth Embodiment

Next, description is given of an information processor 700 according to a fifth embodiment of the present disclosure. It is to be noted that descriptions of configurations denoted by reference numerals common to those of the foregoing embodiments are omitted as appropriate in order to avoid repetitive descriptions.

### [Configurations]

FIG. 47 illustrates an example of a schematic configuration of the information processor 700 according to the present embodiment. The information processor 700 includes the input reception unit 10, the storage unit 20, the signal processing unit 30, the stimulus control unit 40, the stimulus presentation unit 50, and a biological signal detection unit 710. The biological signal detection unit 710 includes the above-described sensor S, and detects a pulse wave, an electrocardiogram, a blood flow, or emotional sweating. In a case where the sensor S detects the pulse wave, the biological signal detection unit 710 functions as a pulse wave detection unit that detects the pulse wave using the sensor S and outputs the pulse wave obtained by the detection. In a case where the sensor S detects the electrocardiogram, the biological signal detection unit 710 functions as an electrocardiogram detection unit that detects the electrocardiogram using the sensor S and outputs the electrocardiogram obtained by the detection. In a case where the sensor S detects the blood flow, the biological signal detection unit 710 functions as a blood flow detection unit that detects the blood flow using the sensor S and outputs the blood flow obtained by the detection. In a case where the sensor S detects the emotional sweating, the biological signal detection unit 710 functions as a sweat detection unit that detects the emotional sweating using the sensor S and outputs the emotional sweating obtained by the detection. The biological signal detection unit 710 outputs signal data obtained by the detection to the signal processing unit 30.

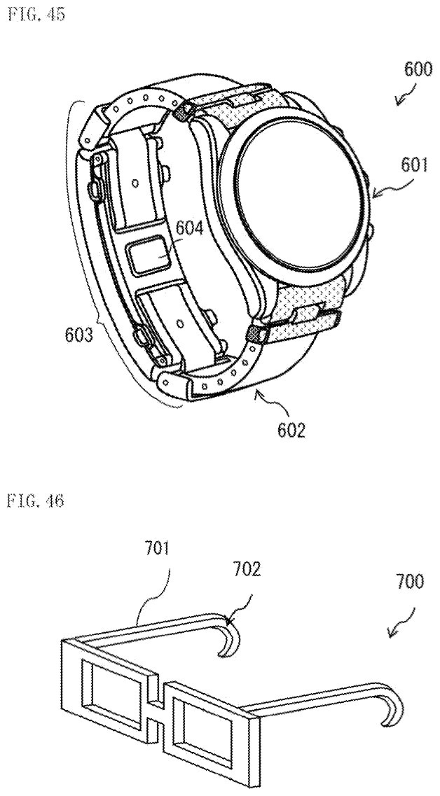

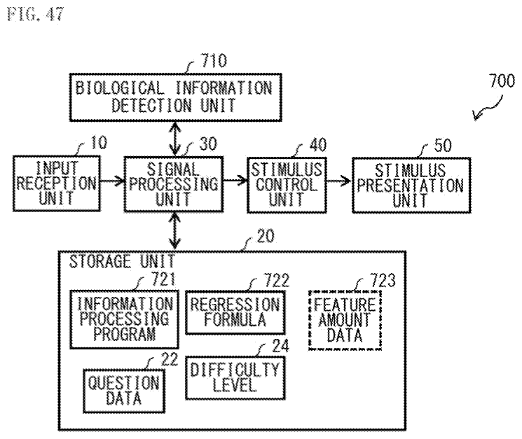

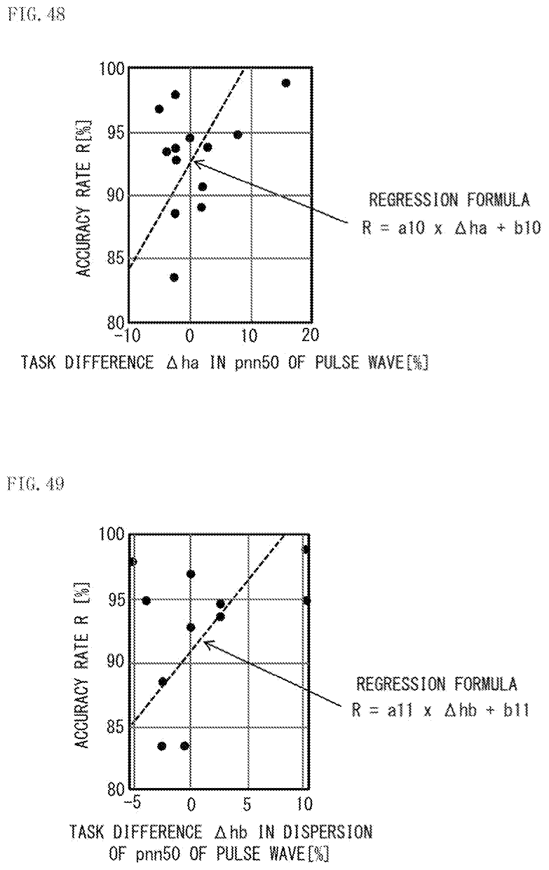

In the information processor 2, the storage unit 20 stores an information processing program 721 to control the cognitive capacity of the user, and the question data 22, a regression formula 722, and the difficulty level 24 which are to be used in the information processing program 721. The regression formula 722 is, for example, a regression formula illustrated in each of FIGS. 48 to 55 described later. The storage unit 20 further stores feature amount data 723 obtained by processing by the information processing program 721.

The signal processing unit 30 executes the information processing program 721 stored in the storage unit 20. The functions of the signal processing unit 30 are implemented, for example, by execution of the information processing program 721 by the signal processing unit 30. For example, the signal processing unit 30 reads question data of a plurality of difficulty levels corresponding to the setting data included in the difficulty level 24 from among the question data 22, and sequentially outputs the read question data of the plurality of difficulty levels to the stimulus control unit 40. The signal processing unit 30 acquires, for example, signal data of the user corresponding to the plurality of pieces of question data of different difficulty levels from the biological signal detection unit 710. On the basis of the acquired signal data, for example, the signal processing unit 30 derives the above-described feature amount (feature amount data 723). For example, when provision of the predetermined number N of questions is completed, the signal processing unit 30 derives the feature amount data 723. On the basis of the derived feature amount data 723 and the regression formula 722 read from the storage unit 20, for example, the signal processing unit 30 derives a cognitive capacity. On the basis of the derived cognitive capacity, for example, the signal processing unit 30 determines the difficulty level of questions to be provided subsequently. On the basis of a table included in the difficulty level 24 read from the storage unit 20, for example, the signal processing unit 30 determines the difficulty level of the questions to be provided subsequently. For example, the signal processing unit 30 writes the determined difficulty levels into the setting data of the storage unit 20 to thereby set the difficulty level of the questions to be provided subsequently.

FIG. 48 illustrates an example of a relationship between a task difference Δha [%] and the accuracy rate R [%]. The task difference Δha [%] is a task difference in the pnn50 of a pulse wave between the time of solving the high difficulty level questions and the time of solving the low difficulty level questions. The accuracy rate R [%] is the accuracy rate at the time of solving the high difficulty level questions. The task difference Δha is obtained by subtracting the pnn50 of the pulse wave at the time of solving the lower-high difficulty level questions from the pnn50 of the pulse wave at the time of solving the high difficulty level questions. In FIG. 48, data for respective users are plotted, and features of the entirety of users are represented by a regression formula (regression line). In FIG. 48, the regression formula is represented by R=a10×Δha+b10.

A small task difference Δha in the pnn50 of the pulse wave means that the difference in the pnn50 of the pulse wave between the time of solving the high difficulty level questions and the time of solving the low difficulty level questions is small. It can be said that a user who has obtained such a result tends to be able to solve questions in a certain range of pnn50 of the pulse wave regardless of the difficulty level of the questions. Meanwhile, a large task difference Δha in the pnn50 of the pulse wave means that the difference in the pnn50 of the pulse wave between the time of solving the high difficulty level questions and the time of solving the low difficulty level questions is large. It can be said that a user who has obtained such a result tends to have a large pnn50 of the pulse wave as the difficulty level of the questions becomes high.

It is appreciated from FIG. 48 that, when the task difference Δha in the pnn50 of the pulse wave is large, the accuracy rate R for questions becomes high, and that, when the task difference Δha in the pnn50 of the pulse wave is small, the accuracy rate R for questions becomes small. It is appreciated from the above that a person who has large pnn50 of the pulse wave for difficult questions tends to have a high accuracy rate R (i.e., be able to answer accurately even for difficult questions to the same degree as for simple questions). Conversely, it is appreciated that a person who has small pnn50 of the pulse wave even for difficult questions tends to have a low accuracy rate R (i.e., the accuracy rate for the difficult questions is lowered).

It can be inferred from the above that, when the task difference Δha in the pnn50 of the pulse wave is large, the cognitive capacity of the user is higher than a predetermined standard. In addition, it can be inferred that, when the task difference Δha in the pnn50 of the pulse wave is small, the cognitive capacity of the user is lower than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δha in the pnn50 of the pulse wave is small, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δha in the pnn50 of the pulse wave is large, raising the difficulty level of the questions may possibly bring the cognitive capacity of the user closer to the predetermined standard.

It is appreciated from the above that using the task difference Δha in the pnn50 of the pulse wave and the regression formula in FIG. 48 makes it possible to control the cognitive capacity of the user.

FIG. 49 illustrates an example of a relationship between a task difference Δhb [%] and the accuracy rate R [%]. The task difference Δhb [%] is a task difference in dispersion of the pnn50 of the pulse wave between the time of solving the high difficulty level questions and the time of solving the low difficulty level questions. The accuracy rate R [%] is the accuracy rate at the time of solving the high difficulty level questions. The task difference Δhb is obtained by subtracting the dispersion of the pnn50 of the pulse wave at the time of solving the lower-high difficulty level questions from the dispersion of the pnn50 of the pulse wave at the time of solving the high difficulty level questions. In FIG. 49, data for respective users are plotted, and features of the entirety of users are represented by a regression formula (regression line). In FIG. 49, the regression formula is represented by R=a11×Δhb+b11.

A small task difference Δhb in the dispersion of the pnn50 of the pulse wave means that the difference in the dispersion of the pnn50 of the pulse wave between the time of solving the high difficulty level questions and the time of solving the low difficulty level questions is small. It can be said that a user who has obtained such a result tends to be able to solve questions in a certain range of dispersion of the pnn50 of the pulse wave regardless of the difficulty level of the questions. Meanwhile, a large task difference Δhb in the dispersion of the pnn50 of the pulse wave means that the difference in the dispersion of the pnn50 of the pulse wave between the time of solving the high difficulty level questions and the time of solving the low difficulty level questions is large. It can be said that a user who has obtained such a result tends to have large dispersion of the pnn50 of the pulse wave as the difficulty level of the questions becomes high.

It is appreciated from FIG. 49 that, when the task difference Δhb in the dispersion of the pnn50 of the pulse wave is large, the accuracy rate R for questions becomes high, and that, when the task difference Δhb in the dispersion of the pnn50 of the pulse wave is small, the accuracy rate R for questions becomes small. It is appreciated from the above that a person who has large dispersion of the pnn50 of the pulse wave for difficult questions tends to have a high accuracy rate R (i.e., be able to answer accurately even for difficult questions to the same degree as for simple questions). Conversely, it is appreciated that a person who has small dispersion of the pnn50 of the pulse wave even for difficult questions tends to have a low accuracy rate R (i.e., the accuracy rate for the difficult questions is lowered).

It can be inferred from the above that, when the task difference Δhb in the dispersion of the pnn50 of the pulse wave is large, the cognitive capacity of the user is higher than a predetermined standard. In addition, it can be inferred that, when the task difference Δha in the dispersion of the pnn50 of the pulse wave is small, the cognitive capacity of the user is lower than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δhb in the dispersion of the pnn50 of the pulse wave is small, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δhb in the dispersion of the pnn50 of the pulse wave is large, raising the difficulty level of the questions may possibly bring the cognitive capacity of the user closer to the predetermined standard.

It is appreciated from the above that using the task difference Δhb in the dispersion of the pnn50 of the pulse wave and the regression formula in FIG. 49 makes it possible to control the cognitive capacity of the user.

FIG. 50 illustrates an example of a relationship between a task difference Δhc [ms' Hz] and the accuracy rate R [%]. The task difference Δhc [ms' Hz] is a task difference in power in a low-frequency band (around 0.01 Hz) of a power spectrum obtained by performing FFT on the pnn50 of the pulse wave between the time of solving the high difficulty level questions and the time of solving the low difficulty level questions. The accuracy rate R [%] is the accuracy rate at the time of solving the high difficulty level questions. Hereinafter, the “power in a low-frequency band (around 0.01 Hz) of a power spectrum obtained by performing FFT on the pnn50 of the pulse wave” is referred to as “power in the low-frequency band of the pnn50 of the pulse wave”. The task difference Δhc is obtained by subtracting the power in the low-frequency band of the pnn50 of the pulse wave at the time of solving the lower-high difficulty level questions from the power in the low-frequency band of the pnn50 of the pulse wave at the time of solving the high difficulty level questions. In FIG. 50, data for respective users are plotted, and features of the entirety of users are represented by a regression formula (regression line). In FIG. 50, the regression formula is represented by R=a12×Δhc+b12.

A small task difference Δhc in the power in the low-frequency band of the pnn50 of the pulse wave means that the difference in the power in the low-frequency band of the pnn50 of the pulse wave between the time of solving the high difficulty level questions and the time of solving the low difficulty level questions is small. It can be said that a user who has obtained such a result tends to be able to solve questions in a certain range of power in the low-frequency band of the pnn50 of the pulse wave regardless of the difficulty level of the questions. Meanwhile, the task difference Δhc in the power in the low-frequency band of the pnn50 of the pulse wave being large in a negative direction means that the difference in the power in the low-frequency band of the pnn50 of the pulse wave between the time of solving the high difficulty level questions and the time of solving the low difficulty level questions is large. It can be said that a user who has obtained such a result tends to have small power in the low-frequency band of the pnn50 of the pulse wave as the difficulty level of the questions becomes high.

It is appreciated from FIG. 50 that, when the task difference Δhc in the power in the low-frequency band of the pnn50 of the pulse wave is small, the accuracy rate R for questions becomes high, and that, when the task difference Δhc in the power in the low-frequency band of the pnn50 of the pulse wave is large in the negative direction, the accuracy rate R for questions becomes small. It is appreciated from the above that a person who has large power in the low-frequency band of the pnn50 of the pulse wave even for difficult questions tends to have a high accuracy rate R (i.e., be able to answer accurately even for difficult questions to the same degree as for simple questions). Conversely, it is appreciated that a person who has small power in the low-frequency band of the pnn50 of the pulse wave for difficult questions tends to have a low accuracy rate R (i.e., the accuracy rate for the difficult questions is lowered).

It can be inferred from the above that, when the task difference Δhc in the power in the low-frequency band of the pnn50 of the pulse wave is small, the cognitive capacity of the user is higher than a predetermined standard. In addition, it can be inferred that, when the task difference Δhc in the power in the low-frequency band of the pnn50 of the pulse wave is large in the negative direction, the cognitive capacity of the user is lower than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δhc in the power in the low-frequency band of the pnn50 of the pulse wave is large in the negative direction, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δhc in the power in the low-frequency band of the pnn50 of the pulse wave is small, raising the difficulty level of the questions may possibly bring the cognitive capacity of the user closer to the predetermined standard.

It is appreciated from the above that using the task difference Δhc in the power in the low-frequency band of the pnn50 of the pulse wave and the regression formula in FIG. 50 makes it possible to control the cognitive capacity of the user.

FIG. 51 illustrates an example of a relationship between a task difference Δhd [ms] and the accuracy rate R [%]. The task difference Δhd [ms] is a task difference in the rmssd of the pulse wave between the time of solving the high difficulty level questions and the time of solving the low difficulty level questions. The accuracy rate R [%] is the accuracy rate at the time of solving the high difficulty level questions. The task difference Δhd is obtained by subtracting the rmssd of the pulse wave at the time of solving the lower-high difficulty level questions from the rmssd of the pulse wave at the time of solving the high difficulty level questions. In FIG. 51, data for respective users are plotted, and features of the entirety of users are represented by a regression formula (regression line). In FIG. 51, the regression formula is represented by R=a13×Δhd+b13.

A small task difference Δhd in the rmssd of the pulse wave means that the difference in the rmssd of the pulse wave between the time of solving the high difficulty level questions and the time of solving the low difficulty level questions is small. It can be said that a user who has obtained such a result tends to be able to solve questions in a certain range of rmssd of the pulse wave regardless of the difficulty level of the questions. Meanwhile, the task difference Δhd in the rmssd of the pulse wave being large in the negative direction means that the difference in the rmssd of the pulse wave between the time of solving the high difficulty level questions and the time of solving the low difficulty level questions is large. It can be said that a user who has obtained such a result tends to have small rmssd of the pulse wave as the difficulty level of the questions becomes high.

It is appreciated from FIG. 51 that, when the task difference Δhd in the rmssd of the pulse wave is small, the accuracy rate R for questions becomes high, and that, when the task difference Δhd in the rmssd of the pulse wave is large in the negative direction, the accuracy rate R for questions becomes small. It is appreciated from the above that a person who has large rmssd of the pulse wave even for difficult questions tends to have a high accuracy rate R (i.e., be able to answer accurately even for difficult questions to the same degree as for simple questions). Conversely, it is appreciated that a person who has small rmssd of the pulse wave for difficult questions tends to have a low accuracy rate R (i.e., the accuracy rate for the difficult questions is lowered).

It can be inferred from the above that, when the task difference Δhd in the rmssd of the pulse wave is small, the cognitive capacity of the user is higher than a predetermined standard. In addition, it can be inferred that, when the task difference Δhd in the rmssd of the pulse wave is large in the negative direction, the cognitive capacity of the user is lower than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δhd in the rmssd of the pulse wave is large in the negative direction, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δhd in the rmssd of the pulse wave is small, raising the difficulty level of the questions may possibly bring the cognitive capacity of the user closer to the predetermined standard.

It is appreciated from the above that using the task difference Δhd in the rmssd of the pulse wave and the regression formula in FIG. 51 makes it possible to control the cognitive capacity of the user.

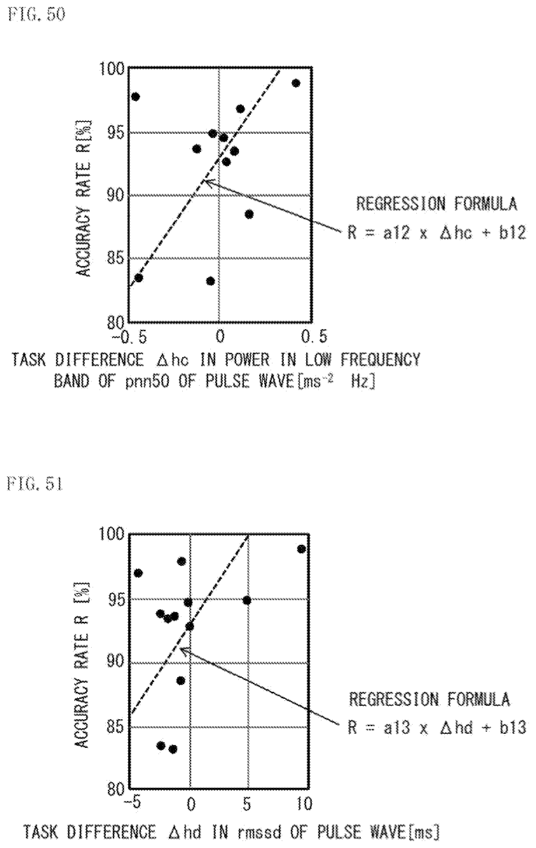

FIG. 52 illustrates an example of a relationship between a task difference Δhe [ms] and the accuracy rate R [%]. The task difference Δhe [ms] is a task difference in dispersion of the rmssd of the pulse wave between the time of solving the high difficulty level questions and the time of solving the low difficulty level questions. The accuracy rate R [%] is the accuracy rate at the time of solving the high difficulty level questions. The task difference Δhe is obtained by subtracting the dispersion of the rmssd of the pulse wave at the time of solving the lower-high difficulty level questions from the dispersion of the rmssd of the pulse wave at the time of solving the high difficulty level questions. In FIG. 52, data for respective users are plotted, and features of the entirety of users are represented by a regression formula (regression line). In FIG. 52, the regression formula is represented by R=a14×Δhe+b14.

A small task difference Δhe in the dispersion of the rmssd of the pulse wave means that the difference in the dispersion of the rmssd of the pulse wave between the time of solving the high difficulty level questions and the time of solving the low difficulty level questions is small. It can be said that a user who has obtained such a result tends to be able to solve questions in a certain range of dispersion of the rmssd of the pulse wave regardless of the difficulty level of the questions. Meanwhile, the task difference Δhe in the dispersion of the rmssd of the pulse wave being large in the negative direction means that the difference in the dispersion of the rmssd of the pulse wave between the time of solving the high difficulty level questions and the time of solving the low difficulty level questions is large. It can be said that a user who has obtained such a result tends to have small dispersion of the rmssd of the pulse wave as the difficulty level of the questions becomes high.

It is appreciated from FIG. 52 that, when the task difference Δhe in the dispersion of the rmssd of the pulse wave is small, the accuracy rate R for questions becomes high, and that, when the task difference Δhe in the dispersion of the rmssd of the pulse wave is large in the negative direction, the accuracy rate R for questions becomes small. It is appreciated from the above that a person who has large dispersion of the rmssd of the pulse wave even for difficult questions tends to have a high accuracy rate R (i.e., be able to answer accurately even for difficult questions to the same degree as for simple questions). Conversely, it is appreciated that a person who has small dispersion of the rmssd of the pulse wave for difficult questions tends to have a low accuracy rate R (i.e., the accuracy rate for the difficult questions is lowered).

It can be inferred from the above that, when the task difference Δhe in the dispersion of the rmssd of the pulse wave is small, the cognitive capacity of the user is higher than a predetermined standard. In addition, it can be inferred that, when the task difference Δhe in the dispersion of the rmssd of the pulse wave is large in the negative direction, the cognitive capacity of the user is lower than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δhe in the dispersion of the rmssd of the pulse wave is large in the negative direction, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δhe in the dispersion of the rmssd of the pulse wave is small, raising the difficulty level of the questions may possibly bring the cognitive capacity of the user closer to the predetermined standard.

It is appreciated from the above that using the task difference Δhe in the dispersion of the rmssd of the pulse wave and the regression formula in FIG. 52 makes it possible to control the cognitive capacity of the user.

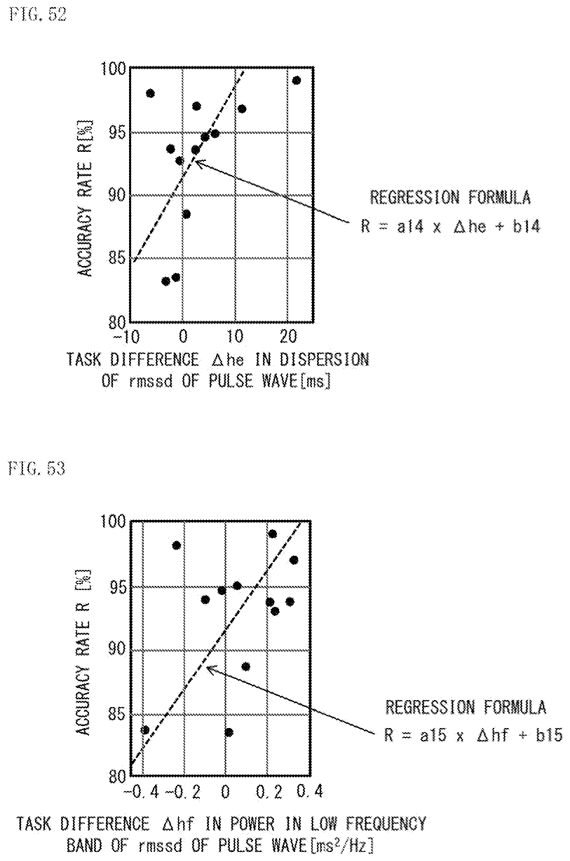

FIG. 53 illustrates an example of a relationship between a task difference Δhf [ms²/Hz] and the accuracy rate R [%]. The task difference Δhf [ms²/Hz] is a task difference in power in a low-frequency band (around 0.01 Hz) of a power spectrum obtained by performing FFT on the rmssd of the pulse wave between the time of solving the high difficulty level questions and the time of solving the low difficulty level questions. The accuracy rate R [%] is the accuracy rate at the time of solving the high difficulty level questions. Hereinafter, the “power in a low-frequency band (around 0.01 Hz) of a power spectrum obtained by performing FFT on the rmssd of the pulse wave” is referred to as “power in the low-frequency band of the rmssd of the pulse wave”. The task difference Δhf is obtained by subtracting the power in the low-frequency band of the rmssd of the pulse wave at the time of solving the lower-high difficulty level questions from the power in the low-frequency band of the rmssd of the pulse wave at the time of solving the high difficulty level questions. In FIG. 53, data for respective users are plotted, and features of the entirety of users are represented by a regression formula (regression line). In FIG. 53, the regression formula is represented by R=a15×Δhf+b15.

A small task difference Δhf in the power in the low-frequency band of the rmssd of the pulse wave means that the difference in the power in the low-frequency band of the rmssd of the pulse wave between the time of solving the high difficulty level questions and the time of solving the low difficulty level questions is small. It can be said that a user who has obtained such a result tends to be able to solve questions in a certain range of power in the low-frequency band of the rmssd of the pulse wave regardless of the difficulty level of the questions. Meanwhile, the task difference Δhf in the power in the low-frequency band of the rmssd of the pulse wave being large in the negative direction means that the difference in the power in the low-frequency band of the rmssd of the pulse wave between the time of solving the high difficulty level questions and the time of solving the low difficulty level questions is large. It can be said that a user who has obtained such a result tends to have small power in the low-frequency band of the rmssd of the pulse wave as the difficulty level of the questions becomes high.

It is appreciated from FIG. 53 that, when the task difference Δhf in the power in the low-frequency band of the rmssd of the pulse wave is small, the accuracy rate R for questions becomes high, and that, when the task difference Δhf in the power in the low-frequency band of the rmssd of the pulse wave is large in the negative direction, the accuracy rate R for questions becomes small. It is appreciated from the above that a person who has large power in the low-frequency band of the rmssd of the pulse wave even for difficult questions tends to have a high accuracy rate R (i.e., be able to answer accurately even for difficult questions to the same degree as for simple questions). Conversely, it is appreciated that a person who has small power in the low-frequency band of the rmssd of the pulse wave for difficult questions tends to have a low accuracy rate R (i.e., the accuracy rate for the difficult questions is lowered).

It can be inferred from the above that, when the task difference Δhf in the power in the low-frequency band of the rmssd of the pulse wave is small, the cognitive capacity of the user is higher than a predetermined standard. In addition, it can be inferred that, when the task difference Δhf in the power in the low-frequency band of the rmssd of the pulse wave is large in the negative direction, the cognitive capacity of the user is lower than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δhf in the power in the low-frequency band of the rmssd of the pulse wave is large in the negative direction, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δhf in the power in the low-frequency band of the rmssd of the pulse wave is small, raising the difficulty level of the questions may possibly bring the cognitive capacity of the user closer to the predetermined standard.

It is appreciated from the above that using the task difference Δhf in the power in the low-frequency band of the rmssd of the pulse wave and the regression formula in FIG. 53 makes it possible to control the cognitive capacity of the user.

FIG. 54 illustrates an example of a relationship between a task difference Δhg [min] and the accuracy rate R [%]. The task difference Δhg [min] is a task difference in dispersion of the number of SCRs of the emotional sweating between the time of solving the high difficulty level questions and the time of solving the low difficulty level questions. The accuracy rate R [%] is the accuracy rate at the time of solving the high difficulty level questions. The task difference Δhg is obtained by subtracting the dispersion of the number of the SCRs of the emotional sweating at the time of solving the lower-high difficulty level questions from the dispersion of the number of SCRs of the emotional sweating at the time of solving the high difficulty level questions. In FIG. 54, data for respective users are plotted, and features of the entirety of users are represented by a regression formula (regression line). In FIG. 54, the regression formula is represented by R=a16×Δhg+b16.

A small task difference Δhg in the dispersion of the number of the SCRs of the emotional sweating means that the difference in the dispersion of the number of the SCRs of the emotional sweating between the time of solving the high difficulty level questions and the time of solving the low difficulty level questions is small. It can be said that a user who has obtained such a result tends to be able to solve questions in a certain range of dispersion of the number of the SCRs of the emotional sweating regardless of the difficulty level of the questions. Meanwhile, the task difference Δhg in the dispersion of the number of the SCRs of the emotional sweating being large in the negative direction means that the difference in the dispersion of the number of the SCRs of the emotional sweating between the time of solving the high difficulty level questions and the time of solving the low difficulty level questions is large. It can be said that a user who has obtained such a result tends to have small dispersion of the number of the SCRs of the emotional sweating as the difficulty level of the questions becomes high.

It is appreciated from FIG. 54 that, when the task difference Δhg in the dispersion of the number of the SCRs of the emotional sweating is small, the accuracy rate R for questions becomes high, and that, when the task difference Δhg in the dispersion of the number of the SCRs of the emotional sweating is large in the negative direction, the accuracy rate R for questions becomes small. It is appreciated from the above that a person who has large dispersion of the number of the SCRs of the emotional sweating even for difficult questions tends to have a high accuracy rate R (i.e., be able to answer accurately even for difficult questions to the same degree as for simple questions). Conversely, it is appreciated that a person who has small dispersion of the number of the SCRs of the emotional sweating for difficult questions tends to have a low accuracy rate R (i.e., the accuracy rate for the difficult questions is lowered).

It can be inferred from the above that, when the task difference Δhg in the dispersion of the number of the SCRs of the emotional sweating is small, the cognitive capacity of the user is higher than a predetermined standard. In addition, it can be inferred that, when the task difference Δhg in the dispersion of the number of the SCRs of the emotional sweating is large in the negative direction, the cognitive capacity of the user is lower than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δhg in the dispersion of the number of the SCRs of the emotional sweating is large in the negative direction, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δhg in the dispersion of the number of the SCRs of the emotional sweating is small, raising the difficulty level of the questions may possibly bring the cognitive capacity of the user closer to the predetermined standard.

It is appreciated from the above that using the task difference Δhgf in the dispersion of the number of the SCRs of the emotional sweating and the regression formula in FIG. 54 makes it possible to control the cognitive capacity of the user.

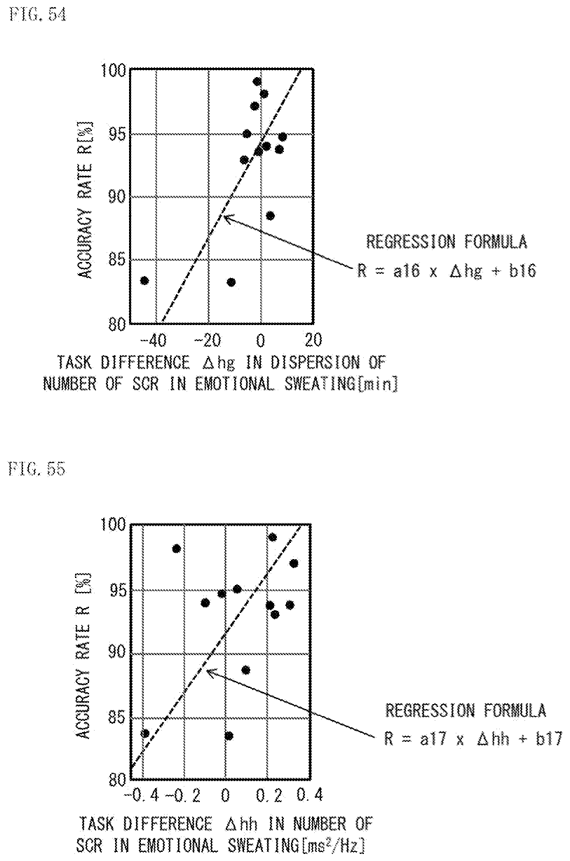

FIG. 55 illustrates an example of a relationship between a task difference Δhh [ms²/Hz] and the accuracy rate R [%]. The task difference Δhh [ms²/Hz] is a task difference in the number of the SCRs of the emotional sweating between the time of solving the high difficulty level questions and the time of solving the low difficulty level questions. The accuracy rate R [%] is the accuracy rate at the time of solving the high difficulty level questions. The task difference Δhh is obtained by subtracting the number of the SCRs of the emotional sweating at the time of solving the lower-high difficulty level questions from the number of SCRs of the emotional sweating at the time of solving the high difficulty level questions. In FIG. 55, data for respective users are plotted, and features of the entirety of users are represented by a regression formula (regression line). In FIG. 55, the regression formula is represented by R=a17×Δhh+b17.

A small task difference Δhh in the number of the SCRs of the emotional sweating means that the difference in the number of the SCRs of the emotional sweating between the time of solving the high difficulty level questions and the time of solving the low difficulty level questions is small. It can be said that a user who has obtained such a result tends to be able to solve questions in a certain range of the number of the SCRs of the emotional sweating regardless of the difficulty level of the questions. Meanwhile, the task difference Δhh in the number of the SCRs of the emotional sweating being large in the negative direction means that the difference in the number of the SCRs of the emotional sweating between the time of solving the high difficulty level questions and the time of solving the low difficulty level questions is large. It can be said that a user who has obtained such a result tends to have the small number of the SCRs of the emotional sweating as the difficulty level of the questions becomes high.

It is appreciated from FIG. 55 that, when the task difference Δhh in the number of the SCRs of the emotional sweating is small, the accuracy rate R for questions becomes high, and that, when the task difference Δhh in the number of the SCRs of the emotional sweating is large in the negative direction, the accuracy rate R for questions becomes small. It is appreciated from the above that a person who has the large number of the SCRs of the emotional sweating even for difficult questions tends to have a high accuracy rate R (i.e., be able to answer accurately even for difficult questions to the same degree as for simple questions). Conversely, it is appreciated that a person who has the small number of the SCRs of the emotional sweating for difficult questions tends to have a low accuracy rate R (i.e., the accuracy rate for the difficult questions is lowered).

It can be inferred from the above that, when the task difference Δhh in the number of the SCRs of the emotional sweating is small, the cognitive capacity of the user is higher than a predetermined standard. In addition, it can be inferred that, when the task difference Δhh in the number of the SCRs of the emotional sweating is large in the negative direction, the cognitive capacity of the user is lower than the predetermined standard. In a case where the cognitive capacity of the user is lower than the predetermined standard, the difficulty level of questions may possibly be too high (i.e., high load) for the user. Meanwhile, in a case where the cognitive capacity of the user is higher than the predetermined standard, the difficulty level of questions may possibly be too low (i.e., low load) for the user.

In a case where the cognitive capacity of the user is lower than the predetermined standard, lowering the difficulty level of the questions may possibly bring the cognitive capacity of the user closer to the predetermined standard. In other words, in a case where the task difference Δhh in the number of the SCRs of the emotional sweating is large in the negative direction, lowering the difficulty level of the questions may possibly bring the cognitive capacity of the user closer to the predetermined standard. In addition, in a case where the cognitive capacity of the user is higher than the predetermined standard, raising the difficulty level of the questions may possibly bring the cognitive capacity of the user closer to the predetermined standard. In other words, in a case where the task difference Δhh in the number of the SCRs of the emotional sweating is small, raising the difficulty level of the questions may possibly bring the cognitive capacity of the user closer to the predetermined standard.

It is appreciated from the above that using the task difference Δhh in the number of the SCRs of the emotional sweating and the regression formula in FIG. 55 makes it possible to control the cognitive capacity of the user.

### [Effects]

Next, description is given of effects of the information processor 700 and the information processing program 721 according to the present embodiment.

In the information processor 700 and the information processing program 721 according to the present embodiment, a plurality of pieces of question data to be presented to the user is determined on the basis of the feature amount data 723 on the user with respect to the question data. Here, the present discloser has experimentally obtained knowledge that the feature amount data 723 on the user varies depending on tasks. It is therefore possible to determine the question data to be presented to the user on the basis of the feature amount data 723. Thus, it is possible to derive the cognitive capacity regardless of whether or not there is reaction time.

In the information processor 700 and the information processing program 721 according to the present embodiment, the cognitive capacity of the user is derived on the basis of the feature amount data 723 on the user with respect to the question data. This makes it possible, on the basis of the derived cognitive capacity, to determine the difficulty level of question data to be presented to the user and to determine subsequent question data. Thus, it is possible to derive the cognitive capacity regardless of whether or not there is reaction time.

### 13. Modification Example of Fifth Embodiment

In the foregoing fifth embodiment, the biological information detection unit 710 may detect biological information (pulse wave, electrocardiogram, blood flow, or emotional sweating) on a plurality of users. In this case, on the basis of signal data of the biological information (pulse wave, electrocardiogram, blood flow, or emotional sweating) obtained from each of the users, the signal processing unit 30 derives the feature amount data 723 for each of the users. On the basis of the derived feature amount data 723 and the regression formula 722 read from the storage unit 20, the signal processing unit 30 derives a cognitive capacity for each of the users. On the basis of the cognitive capacity derived for each of the users, the signal processing unit 30 derives a cognitive capacity of a group when the plurality of users is viewed as a group. In such a case, it is possible to determine, for example, how much load a task causes on the group or how much margin the group has with respect to the task.

### 14. Modification Examples of First to Fifth Embodiments

Next, description is given of modification examples of the information processors 1 to 4 and 700 according to the first to fifth embodiments.

### Modification Example H

In the information processor 2 according to the second embodiment, for example, as illustrated in FIG. 56, the biological information detection unit 60 may be provided separately from the information processor 2. In this case, for example, the signal processing unit 30 may communicate with the biological information detection unit 60 via a communication unit 70.

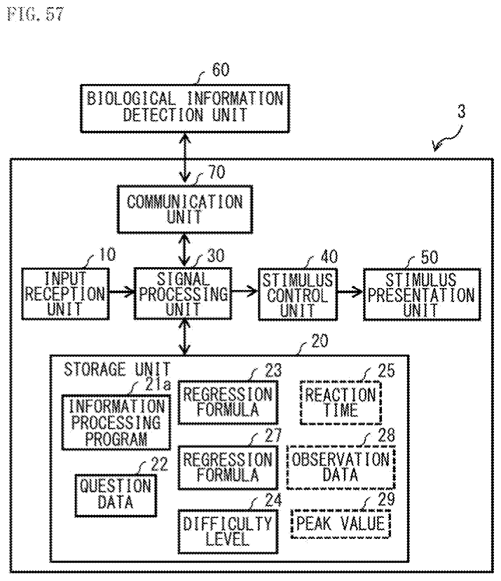

In addition, in the information processor 3 according to the third embodiment, for example, as illustrated in FIG. 57, the biological information detection unit 60 may be provided separately from the information processor 3. In this case, for example, the signal processing unit 30 may communicate with the biological information detection unit 60 via the communication unit 70.

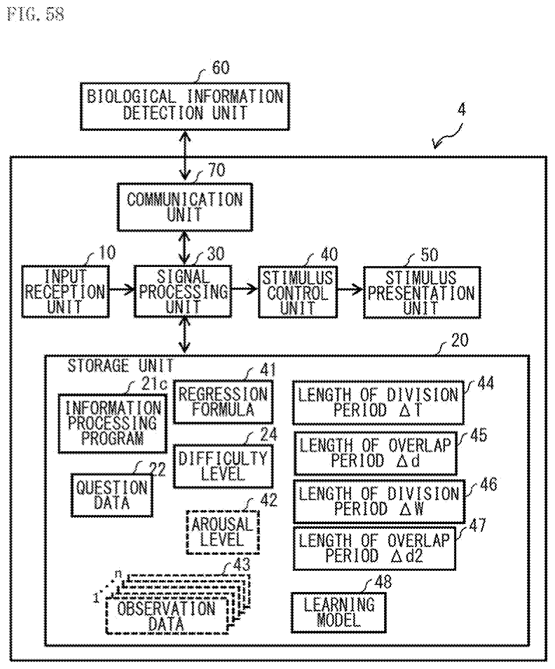

In addition, in the information processor 4 according to the fourth embodiment, for example, as illustrated in FIG. 58, the biological information detection unit 60 may be provided separately from the information processor 4. In this case, for example, the signal processing unit 30 may communicate with the biological information detection unit 60 via the communication unit 70.

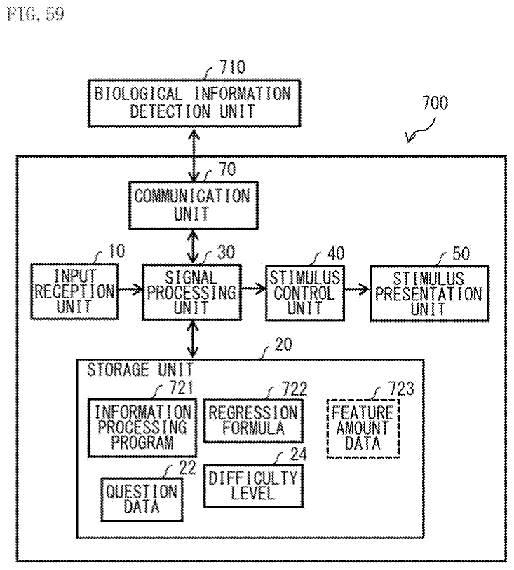

In addition, in the information processor 700 according to the fifth embodiment, for example, as illustrated in FIG. 59, the biological signal detection unit 710 may be provided separately from the information processor 700. In this case, for example, the signal processing unit 30 may communicate with the biological signal detection unit 710 via the communication unit 70.

### Modification Example I

In the information processor 1 according to the first embodiment, some of the functions of each of the information processing programs 21, 21a, 21b, and 21g may be performed by an external apparatus configured to be able to communicate with the information processor 1. In this case, for example, as illustrated in FIG. 60, an information processing system 5 includes the information processor 1 and a server apparatus 6 that are configured to be able to communicate with each other.

In the present modification example, the information processor 1 includes the storage unit 20 that stores an information processing program 21A. The information processing program 21A includes a series of procedures to cause the signal processing unit 30 to execute some of the functions of each of the information processing programs 21, 21a, 21b, and 21g. In FIG. 11, the information processing program 21A includes, for example, a series of procedures until the reaction time 25 is calculated (acquired). When the information processing program 21A is loaded, the signal processing unit 30 executes the steps S101, S102, S121, S122, S141, S142, S161, and S162 in each of FIGS. 11, 15, 20, 23, and 39 to thereby calculate (acquire) the reaction time 25. The signal processing unit 30 transmits the calculated (acquired) reaction time 25 to the server apparatus 6 via a communication unit 80. The communication unit 80 is configured to be able to communicate with the server apparatus 6.

In the present modification example, the server apparatus 6 includes, for example, a control unit 61, a communication unit 62, and a storage unit 63. The communication unit 62 is configured to be able to communicate with the information processor 1 (communication unit 80). The storage unit 63 is, for example, a volatile memory such as a DRAM, or a non-volatile memory such as an EEPROM or a flash memory. The storage unit 63 stores an information processing program 63A to control the cognitive capacity of the user, and the question data 22, the regression formula 23, and the difficulty level 24 which are to be used in the information processing program 63A. The information processing program 63A includes, for example, a series of procedures to be executed by the signal processing unit 30 in the information processing programs 21, 21a, 21b, and 21g, excluding the series of procedures until the reaction time 25 is calculated (acquired). When the information processing program 63A is loaded, the control unit 61 executes the series of procedures to be executed by the signal processing unit 30 in the information processing programs 21, 21a, 21b, and 21g, excluding the series of procedures until the reaction time 25 is calculated (acquired). Thus, the control unit 61 derives a cognitive capacity of the user, determines a difficulty level of question data on the basis of the derived cognitive capacity, and writes the determined difficulty level in the setting data in the difficulty level 24 of the storage unit 63, to thereby set the difficulty level of questions to be provided subsequently.

In the present modification example, some of the functions of the information processing programs 21, 21a, 21b, and 21g are performed by the external apparatus configured to be able to communicate with the information processor 1. Also in such a case, similarly to the information processor 1 according to the first embodiment, it is possible to derive the cognitive capacity regardless of whether or not there is correct reaction time.

### Modification Example J

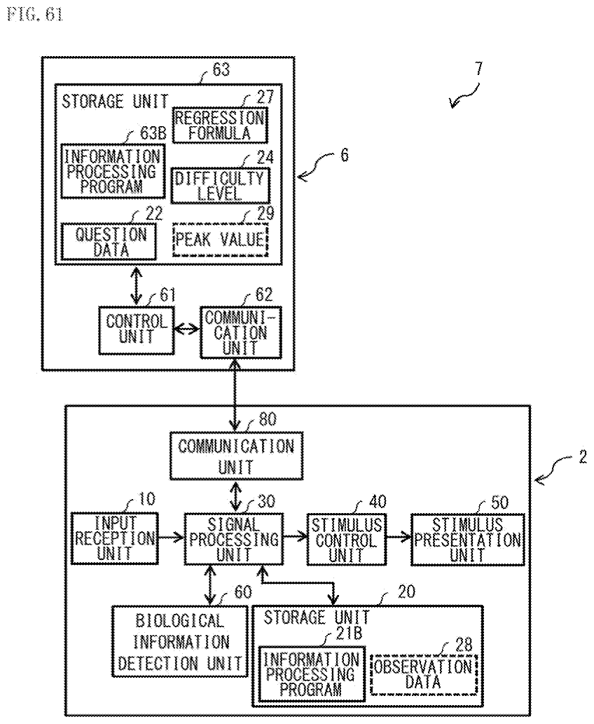

In the information processor 2 according to the second embodiment, some of functions of the information processing program 21c may be performed by an external apparatus configured to be able to communicate with the information processor 2. In this case, for example, as illustrated in FIG. 61, an information processing system 7 includes the information processor 2 and the server apparatus 6 that are configured to be able to communicate with each other.

In the present modification example, the information processor 2 includes the storage unit 20 that stores an information processing program 21B. The information processing program 21B includes a series of procedures to cause the signal processing unit 30 to execute some of the functions of the information processing program 21c. In FIG. 26, the information processing program 21B includes, for example, a series of procedures until the observation data 28 is acquired. When the information processing program 21B is loaded, the signal processing unit 30 executes the steps S201 and S202 in FIG. 26 to thereby acquire the observation data 28. The signal processing unit 30 transmits the acquired observation data 28 to the server apparatus 6 via the communication unit 80.

In the present modification example, the server apparatus 6 includes, for example, the control unit 61, the communication unit 62, and the storage unit 63. The communication unit 62 is configured to be able to communicate with the information processor 2 (communication unit 80). The storage unit 63 stores an information processing program 63B to control the cognitive capacity of the user, and the question data 22, a regression formula 27, and the difficulty level 24 which are to be used in the information processing program 63B. The information processing program 63B includes, for example, a series of procedures to be executed by the signal processing unit 30 in the information processing program 21c, excluding the series of procedures until the observation data 28 is acquired. When the information processing program 63B is loaded, the control unit 61 executes the series of procedures to be executed by the signal processing unit 30 in the information processing program 21c, excluding the series of procedures until the observation data 28 is acquired. Thus, the control unit 61 derives a cognitive capacity of the user, determines a difficulty level of question data on the basis of the derived cognitive capacity, and writes the determined difficulty level in the setting data in the difficulty level 24 of the storage unit 63, to thereby set the difficulty level of questions to be provided subsequently.

In the present modification example, some of the functions of the information processing program 21c are performed by the external apparatus configured to be able to communicate with the information processor 2. Also in such a case, similarly to the information processor 2 according to the second embodiment, it is possible to derive the cognitive capacity regardless of whether or not there is reaction time.

### Modification Example K

In the information processor 3 according to the third embodiment, some of the functions of the information processing program 21 may be performed by an external apparatus configured to be able to communicate with the information processor 3. In this case, for example, as illustrated in FIG. 62, an information processing system 8 includes the information processor 3 and the server apparatus 6 that are configured to be able to communicate with each other.

In the present modification example, the information processor 3 includes the storage unit 20 that stores an information processing program 21C. The information processing program 21C includes a series of procedures to cause the signal processing unit 30 to execute some of functions of the information processing program 21d. In FIG. 29, the information processing program 21C includes, for example, a series of procedures until the reaction time 25 and the observation data 28 are acquired. When the information processing program 21C is loaded, the signal processing unit 30 acquires the reaction time 25 and the observation data 28. The signal processing unit 30 transmits the acquired reaction time 25 and the observation data 28 to the server apparatus 6 via the communication unit 80.

In the present modification example, the server apparatus 6 includes, for example, the control unit 61, the communication unit 62, and the storage unit 63. The communication unit 62 is configured to be able to communicate with the information processor 3 (communication unit 80). The storage unit 63 stores an information processing program 63C to control the cognitive capacity of the user, and the question data 22, the regression formulae 23 and 27, and the difficulty level 24 which are to be used in the information processing program 63C. The information processing program 63C includes, for example, a series of procedures to be executed by the signal processing unit 30 in the information processing program 21d, excluding the series of procedures until the reaction time 25 and the observation data 28 are acquired. When the information processing program 63C is loaded, the control unit 61 executes the series of procedures to be executed by the signal processing unit 30 in the information processing program 21d, excluding the series of procedures until the reaction time 25 and the observation data 28 are acquired. Thus, the control unit 61 derives a cognitive capacity of the user, determines a difficulty level of question data on the basis of the derived cognitive capacity, and writes the determined difficulty level in the setting data in the difficulty level 24 of the storage unit 63, to thereby set the difficulty level of questions to be provided subsequently.

In the present modification example, some of the functions of the information processing program 21d are performed by the external apparatus configured to be able to communicate with the information processor 3. Also in such a case, similarly to the information processor 3 according to the third embodiment, it is possible to derive the cognitive capacity regardless of whether or not there is reaction time.

### Modification Example L

In the information processor 4 according to the fourth embodiment, some of functions of the information processing program 21e may be performed by an external apparatus configured to be able to communicate with the information processor 4. In this case, for example, as illustrated in FIG. 63, an information processing system 9 includes the information processor 4 and the server apparatus 6 that are configured to be able to communicate with each other.

In the present modification example, the information processor 4 includes the storage unit 20 that stores an information processing program 21D. The information processing program 21D includes a series of procedures to cause the signal processing unit 30 to execute some of the functions of the information processing program 21e. In FIG. 33, the information processing program 21D includes, for example, a series of procedures until the observation data 43 is acquired. When the information processing program 21D is loaded, the signal processing unit 30 executes the steps S401 and S402 in FIG. 33 to thereby acquire the observation data 43. The signal processing unit 30 transmits the acquired observation data 43 to the server apparatus 6 via the communication unit 80.

In the present modification example, the server apparatus 6 includes, for example, the control unit 61, the communication unit 62, and the storage unit 63. The communication unit 62 is configured to be able to communicate with the information processor 4 (communication unit 80). The storage unit 63 stores an information processing program 63D to control the cognitive capacity of the user, and the question data 22, the regression formula 41, the difficulty level 24, the length 44 of the division period ΔT, the length 45 of the overlap period Δd1, the length 46 of the division period ΔW, and the length 47 of the overlap period Δd2 which are to be used in the information processing program 63D. The information processing program 63D includes, for example, a series of procedures to be executed by the signal processing unit 30 in the information processing program 21e, excluding the series of procedures until the observation data 43 is acquired. When the information processing program 63D is loaded, the control unit 61 executes the series of procedures to be executed by the signal processing unit 30 in the information processing program 21e, excluding the series of procedures until the observation data 43 is acquired. Thus, the control unit 61 derives a cognitive capacity of the user, determines a difficulty level of question data on the basis of the derived cognitive capacity, and writes the determined difficulty level in the setting data in the difficulty level 24 of the storage unit 63, to thereby set the difficulty level of questions to be provided subsequently.

In the present modification example, some of the functions of the information processing program 21e are performed by the external apparatus configured to be able to communicate with the information processor 4. Also in such a case, similarly to the information processor 4 according to the fourth embodiment, it is possible to derive the cognitive capacity regardless of whether or not there is reaction time.

### Modification Example M

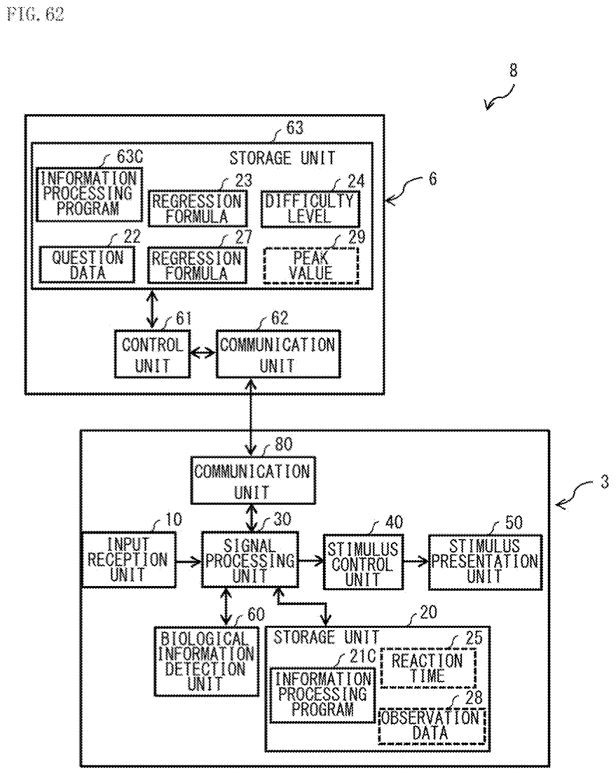

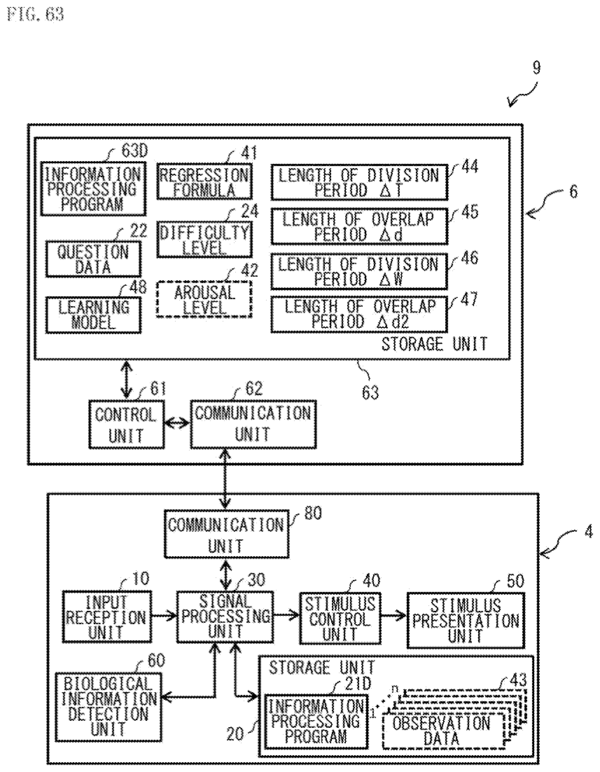

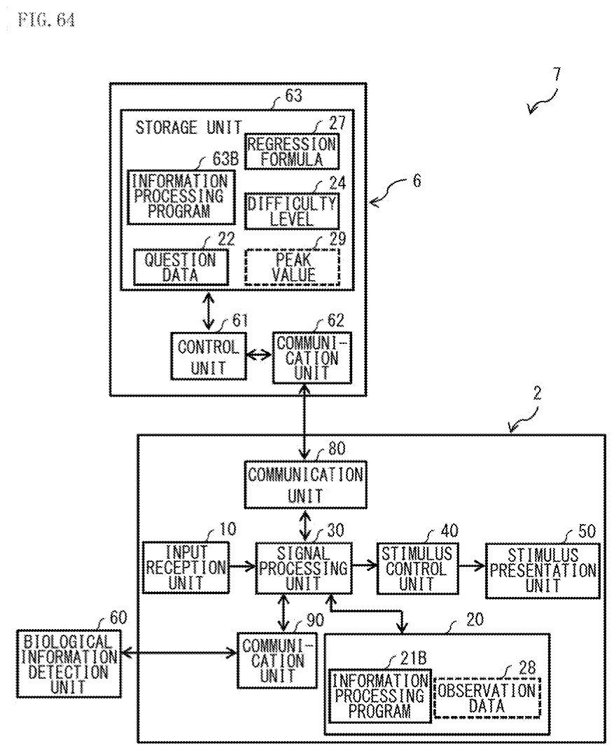

In the foregoing Modification Example J, for example, as illustrated in FIG. 64, the biological information detection unit 60 may be provided separately from the information processor 2. In this case, for example, the signal processing unit 30 may communicate with the biological information detection unit 60 via a communication unit 90.

### Modification Example N

In the foregoing Modification Example K, for example, as illustrated in FIG. 65, the biological information detection unit 60 may be provided separately from the information processor 3. In this case, for example, the signal processing unit 30 may communicate with the biological information detection unit 60 via the communication unit 90.

### Modification Example O

In the Foregoing Modification example L, for example, as illustrated in FIG. 66, the biological information detection unit 60 may be provided separately from the information processor 4. In this case, for example, the signal processing unit 30 may communicate with the biological information detection unit 60 via the communication unit 90.

### Modification Example P

In the foregoing first embodiment, for example, game data 49 illustrated in FIG. 67 may be provided instead of the question data 22. In this case, examples of the reaction of the user include inputting a reaction corresponding to the game data 49 into the input reception unit 10. At this time, the input reception unit 10 receives the input from the user as the reaction corresponding to the game data 49, and outputs the received reaction to the signal processing unit 30.

The game data 49 includes a plurality of pieces of game data of different difficulty levels. The game data corresponds to a specific example of each of the “request” and the “task” of the present disclosure. The game data 49 also includes data on difficulty levels of respective pieces of game data included in the game data 49. The game data 49 may further include correct answer data for each of the pieces of game data.

The difficulty level 24 includes, for example, data for setting the difficulty level of games to be provided to the user, and a table describing a correspondence relationship between the difficulty level of the games and the cognitive capacity of the user. The setting data included in the difficulty level 24 concerns a plurality of difficulty levels as initial values or a plurality of difficulty levels after having been changed by processing by the information processing program 21. The table included in the difficulty level 24 has difficulty levels set in accordance with the cognitive capacity of the user. For example, the table included in the difficulty level 24 has a plurality of difficulty levels, which are set as difficulty levels corresponding to the cognitive capacity a, in a case where the cognitive capacity of the user is α. The setting of the plurality of difficulty levels as difficulty levels corresponding to the cognitive capacity a enables the information processing program 21 to provide the user with games of the plurality of difficulty levels.

For example, the signal processing unit 30 reads game data of the plurality of difficulty levels corresponding to the setting data included in the difficulty level 24 from among the game data 49, and sequentially outputs the read game data of the plurality of difficulty levels to the stimulus control unit 40. When acquiring a reaction corresponding to the game data 49 from the input reception unit 10, for example, the signal processing unit 30 derives the reaction time 25 on the basis of an input timing of the acquired reaction. For example, when provision of game data of a predetermined number N of reactions is completed, the signal processing unit 30 calculates the task difference Δtv in dispersion of the reaction times 25. The signal processing unit 30 derives a cognitive capacity using, for example, the calculated task difference Δtv and the regression formula 23 read from the storage unit 20. The signal processing unit 30 determines difficulty levels of games to be provided subsequently using, for example, a table included in the difficulty level 24 read from the storage unit 20. For example, the signal processing unit 30 writes the determined difficulty levels into the setting data of the storage unit 20 to thereby set the difficulty levels of the games to be provided subsequently.

The stimulus control unit 40 generates a control signal to control the stimulus presentation unit 50 on the basis of the game data inputted from the signal processing unit 30. The stimulus control unit 40 outputs the generated control signal to the stimulus presentation unit 50. In a case where the stimulus presentation unit 50 is a display panel, the stimulus control unit 40 generates, as a control signal, an image signal to display the game data inputted from the signal processing unit 30. The stimulus presentation unit 50 presents a stimulus to the user on the basis of the control signal inputted from the stimulus control unit 40. In a case where the stimulus presentation unit 50 is a display panel, the stimulus presentation unit 50 presents, to the user, an image including the plurality of pieces of game data of different difficulty levels on the basis of the image signal inputted from the stimulus control unit 40.

Next, description is given of operations of the information processor 1 according to the present modification example.

First, the signal processing unit 30 reads game data of a plurality of difficulty levels corresponding to the setting data included in the difficulty level 24 from among the game data 49, and sequentially outputs the read game data of the plurality of difficulty levels to the stimulus control unit 40. On the basis of the game data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on game data of a predetermined difficulty level. The stimulus presentation unit 50 presents to the user, for example, an image including game data. The user then inputs a reaction corresponding to the game data into the input reception unit 10. When acquiring the reaction corresponding to the game data from the user, the input reception unit 10 outputs the acquired reaction to the signal processing unit 30. When acquiring the reaction from the input reception unit 10, the signal processing unit 30 calculates (acquires) the reaction time 25 for the reaction corresponding to the game data.

The signal processing unit 30 executes the above-described series of processing until provision of games of the predetermined number N of reactions is completed. When the provision of the games of the predetermined number N of reactions is completed, the signal processing unit 30 calculates the task difference Δtv in the dispersion of the reaction times 25 acquired thus far. The signal processing unit 30 derives a cognitive capacity using the calculated task difference Δtv and the regression formula 23 read from the storage unit 20. The signal processing unit 30 determines difficulty levels of games to be provided subsequently using the table included in the difficulty level 24 read from the storage unit 20. The signal processing unit 30 writes the determined difficulty levels into the setting data of the storage unit 20 to thereby change the difficulty levels of games to be provided subsequently.

The signal processing unit 30 executes the above-described series of processing until the provision of the games of the predetermined number of reactions is completed. When the provision of the games of the predetermined number of reactions is completed, the signal processing unit 30 finishes the provision of the game.

Next, description is given of a procedure to derive the regression formula 23 in the information processor 1 according to the present modification example. First, the signal processing unit 30 reads game data of a plurality of difficulty levels corresponding to the setting data included in the difficulty level 24 from among the game data 49, and sequentially outputs the read game data of the plurality of difficulty levels to the stimulus control unit 40. On the basis of the game data inputted from the signal processing unit 30, the stimulus control unit 40 generates a control signal to control the stimulus presentation unit 50, and outputs the generated control signal to the stimulus presentation unit 50.

On the basis of the control signal inputted from the stimulus control unit 40, the stimulus presentation unit 50 presents to the user a stimulus based on the game data of the predetermined difficulty level. The stimulus presentation unit 50 presents to the user, for example, an image including game data. The user then inputs a reaction corresponding to the game data into the input reception unit 10. The input reception unit 10 acquires the reaction corresponding to game data from the user. The input reception unit 10 outputs the acquired reaction to the signal processing unit 30. When acquiring the reaction from the input reception unit 10, the signal processing unit 30 determines right or wrong of the reaction corresponding to the game data using correct answer data included in the game data 49. The signal processing unit 30 calculates (acquires) the reaction time 25 and the accuracy rate for the reaction corresponding to the game data.

The signal processing unit 30 executes the above-described series of processing until the provision of the games of the predetermined number N of reactions is completed. When the provision of the games of the predetermined number N of reactions is completed, the signal processing unit 30 calculates the task difference Δtv in the dispersion of the reaction times 25 acquired thus far and the task difference ΔR in the accuracy rate for reactions acquired thus far. On the basis of the calculated task differences Δtv and ΔR, the signal processing unit 30 derives the regression formula 23, and stores the derived regression formula 23 in the storage unit 20.

The information processor 1 may perform the series of procedures to derive the regression formula 23, separately (i.e., in advance) from the series of procedures to change the difficulty levels in the information processor 1. At this time, the user who reacts to the games to derive the regression formula 23 and the user who reacts to the games in the series of procedures to change the difficulty levels in the information processor 1 may be the same as or different from each other. It is to be noted that the information processor 1 may perform the series of procedures for deriving the regression formula 23 to be mixed into the series of procedures to change the difficulty levels in the information processor 1.

In the information processor 1 and the information processing program 21 according to the present modification example, the difficulty levels of the plurality of pieces of game data to be presented subsequently is determined on the basis of the cognitive capacity of the user obtained on the basis of the dispersion of the reaction times 25 corresponding to the plurality of pieces of game data of different difficulty levels. Here, the present discloser has experimentally obtained knowledge that the dispersion of the reaction times 25 varies depending on tasks. It is therefore possible to derive the cognitive capacity of the user on the basis of the dispersion of the reaction times 25, and thus to determine the difficulty levels of the plurality of pieces of game data to be provided subsequently. Thus, it is possible to derive the cognitive capacity regardless of whether or not there is correct reaction time.

### Modification Example Q

In the foregoing embodiments and modification examples thereof, for example, the game data 49 illustrated in FIG. 67 may be provided instead of the question data 22. Also in such cases, it is possible to derive the cognitive capacity regardless of whether or not there is correct reaction time, or regardless of whether or not there is reaction time.

### Modification Example R

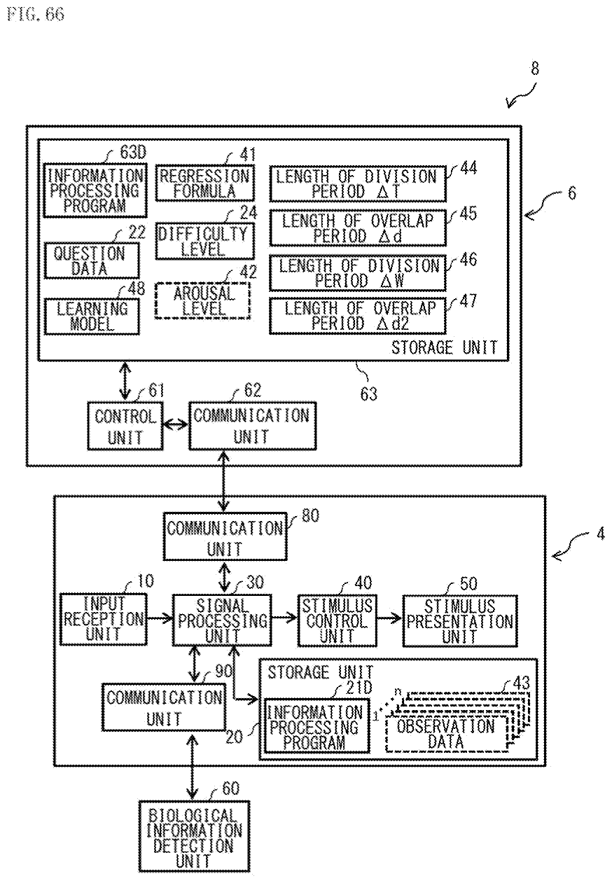

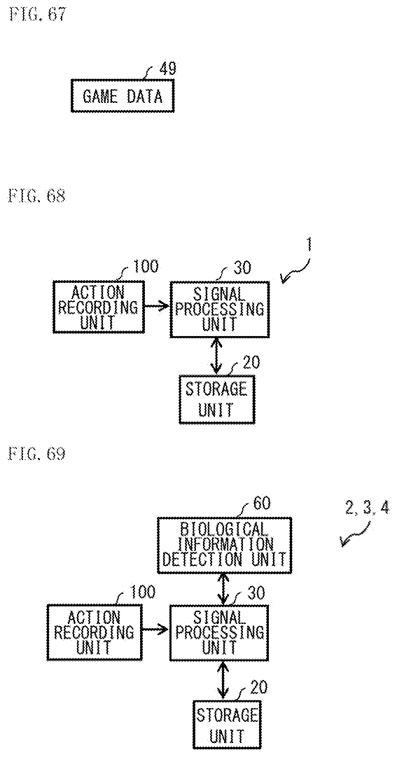

In the foregoing first embodiment and modification examples thereof, for example, as illustrated in FIG. 68, an action recording unit 100 may be provided instead of the input reception unit 10. In addition, in the foregoing second, third, fourth and fifth embodiments and modification examples thereof, for example, as illustrated in FIGS. 69, 70, 71 and 72, the action recording unit 100 may be provided instead of the input reception unit 10.

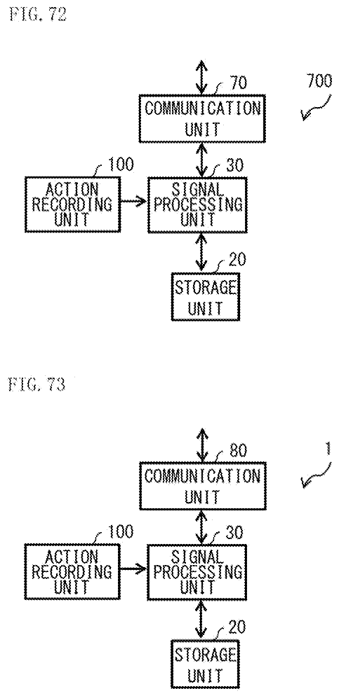

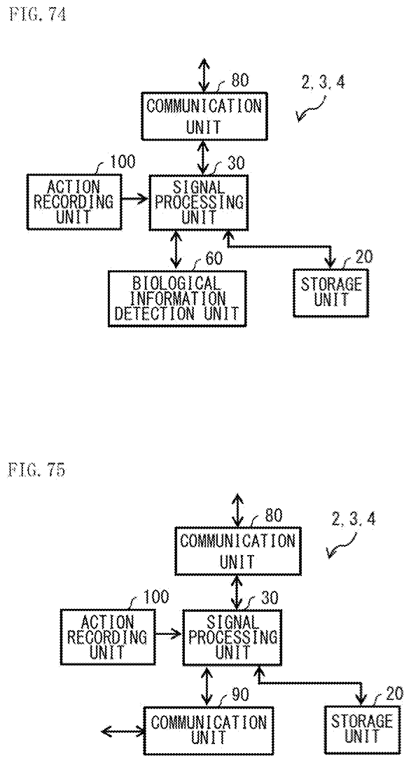

In addition, in the foregoing Modification Example I, for example, as illustrated in FIG. 73, the action recording unit 100 may be provided instead of the input reception unit 10. In addition, in the foregoing Modification Examples J, K, and L, for example, as illustrated in FIG. 74, the action recording unit 100 may be provided instead of the input reception unit 10. In addition, in the foregoing Modification Examples M, N, and 0, for example, as illustrated in FIG. 75, the action recording unit 100 may be provided instead of the input reception unit 10.

The action recording unit 100 acquires an action log of the user. The action recording unit 100 is configured, for example, by a camera, and outputs to the signal processing unit 30, for example, an image (successive still image or moving image) obtained by capturing, using a camera, a user and a question sheet provided to the user. The signal processing unit 30 derives the reaction time 25 on the basis of the acquired action log. The signal processing unit 30 derives the reaction time 25 on the basis of the image (successive still image or moving image) inputted from the action recording unit 100, for example.

The action recording unit 100 may detect, for example, an answer operation and an input operation of the user, which are pieces of information corresponding to a stop of a stopwatch, upon measurement of the reaction time 25. For example, the action recording unit 100 may detect an operation to click a button, a manipulation of a game controller, or a head movement by a head-mounted display. The action recording unit 100 may track the movement of eyeballs using an image sensor, for example.

In the present modification example, the action recording unit 100 is provided instead of the input reception unit 10. Also in such a case, it is possible to derive the reaction time 25 on the basis of the image (successive still image or moving image) inputted from the action recording unit 100. Thus, similarly to the foregoing embodiments and modification examples thereof, it is possible to derive the cognitive capacity regardless of whether or not there is correct reaction time, or regardless of whether or not there is reaction time.

It is to be noted that the effects described herein are merely illustrative. The effects of the present disclosure are not limited to those described herein. The present disclosure may also have effects other than those described herein.

For example, the above-described series of processing may be executed by software or may be executed by hardware.

In addition, in the foregoing plurality of embodiments and modification examples thereof, the signal processing unit 30 determine the difficulty level of questions using the difficulty level 24; the determination also includes determination (selection) of questions per se to be provided subsequently. Accordingly, in the foregoing plurality of embodiments and modification examples thereof, the signal processing unit 30 determines the difficulty level of the questions using the difficulty level 24, and determines (selects) questions corresponding to the determined difficulty level from among the question data 22.

In addition, in the foregoing plurality of embodiments and modification examples thereof, questions to be reacted (answered) by the user and questions to be determined (selected) using the difficulty level 24 may be pieces of data belonging to a field common to each other. In addition, in Modification Example N, games to be reacted (answered) by the user and games to be determined (selected) using the difficulty level 24 may be pieces of data belonging to a field common to each other. For example, questions to be reacted (answered) by the user and questions to be determined (selected) using the difficulty level 24 may correspond to question data in learning of a particular subject.

It is to be noted that, in the foregoing plurality of embodiments and modification examples thereof, questions to be reacted (answered) by the user and questions to be determined (selected) using the difficulty level 24 may be pieces of data belonging to a field different from each other. In addition, in Modification Example N, games to be reacted (answered) by the user and games to be determined (selected) using the difficulty level 24 may be pieces of data belonging to a field different from each other. For example, the questions to be reacted (answered) by the user may be questions of solving puzzles, and the questions to be determined (selected) using the difficulty level 24 may be games of a difficulty level to be determined (selected) using the difficulty level 24.

In addition, the foregoing embodiments and modification examples thereof are applicable to an applications that requires an objective cognitive capacity, for example, in games, healthcare, learning, training for sports games, training for human resource development, and the like. At this time, in the foregoing embodiments and modification examples thereof, at least one of game data, item data in the healthcare, item data in the training for sports games, or item data in the training for human resource development may be used instead of the question data 22 (question data in learning). That is, the present disclosure is applicable to various fields. It is to be noted that at least one of the game data, the item data in the healthcare, the item data in the training for sports games, or the item data in the training for human resource development corresponds to a specific example of each of the “request” and the “task” of the present disclosure.

In addition, in the foregoing plurality of embodiments and modification examples thereof, a detection unit that detects a biological signal other than a brain wave may be provided instead of the biological information detection unit 60. In addition, in the foregoing plurality of embodiments and modification examples thereof, a target to be measured may be a living being (e.g., an animal or the like) other than a human being.

In addition, in the regression formula according to any of the foregoing plurality of embodiments and modification examples thereof, for example, as illustrated in FIG. 76, a task difference Δtv in a median value (median) of reaction times may be used instead of the task difference Δtv in the dispersion of the reaction times.

In addition, in the foregoing plurality of embodiments and modification examples thereof, the regression formula is not limited to a straight line (regression line), but may be a curve (regression curve), for example. The curve (regression curve) may be, for example, a quadratic function. The regression formula defining the relationship between the arousal level k [%] and the accuracy rate R [%] may be defined as a quadratic function (R=a×k²+bk+c), for example, as illustrated in FIG. 77.

In addition, for example, the present disclosure may have the following configurations.

(1-1)

An information processor including a determination unit that determines a task for a user on a basis of a cognitive capacity (cognitive resource) of a user obtained on a basis of dispersion of reaction times of the user for a plurality of requests.

(1-2)

An information processor including a determination unit that determines a task for a user on a basis of dispersion of reaction times of the user for a plurality of requests.

(1-3)

An information processor including a changing unit that changes a task for a user on a basis of dispersion of reaction times of the user for a plurality of requests.

(2)

The information processor according to any one of (1-1), (1-2), and (1-3), further including an acquisition unit that acquires the reaction times.

(3)

The information processor according to (2), in which the acquisition unit acquires the reaction times on a basis of information from a sensor.

(4)

The information processor according to any one of (1-1), (1-2), (1-3), (2), and (3), further including a deriving unit that derives the cognitive capacity of the user on a basis of the dispersion of the reaction times.

(5)

The information processor according to (3), in which the deriving unit derives the cognitive capacity on a basis of a task difference in the dispersion of the reaction times and regression data on the task difference in the dispersion of the reaction times.

(6)

The information processor according to (1-1), in which the deriving unit derives the cognitive capacity on a basis of the dispersion of the reaction times and regression data on the dispersion of the reaction times.

(7)

The information processor according to (1-3), further including a determination unit that determines a task for the user on a basis of the cognitive capacity.

(8)

The information processor according to (7), in which the determination unit changes the task for the user on a basis of the cognitive capacity.

(9)

The information processor according to (2), in which the acquisition unit derives the reaction times on a basis of answer input timings or action logs of the user for the plurality of requests.

(10)

The information processor according to any one of (1-1), (1-2), (1-3), (2), (3), (4), (5), (6), (7), (8), and (9), further including a presentation unit that presents the plurality of requests.

(11)

The information processor according to (10), in which the determination unit determines a plurality of subsequent requests on a basis of the dispersion of the reaction times.

(12)

The information processor according to (1-1), in which the determination unit determines a difficulty level of the task on a basis of the dispersion of the reaction times.

(13)

The information processor according to (12), in which the determination unit changes the difficulty level of the task on a basis of the dispersion of the reaction times.

(14)

The information processor according to any one of (1-1), (1-2), (1-3), (2), (3), (4), (5), (6), (7), (8), (9), (10), (11), (12), and (13), in which the requests correspond to game data, item data in healthcare, question data in learning, item data in training for a sports game, or item data in training for human resource development.

(15-1)

An information processor including a deriving unit that derives a cognitive capacity of a user on a basis of a biological signal of the user for a request.

(15-2)

An information processor including a determination unit that determines a task for a user on a basis of a biological signal of the user for a request.

(16)

The information processor according to any one of (15-1) and (15-2), in which the biological signal includes time series data.

(17)

The information processor according to (16), in which the deriving unit derives the cognitive capacity of the user on a basis of a fluctuation in a component in a specific frequency band included in the biological signal.

(18)

The information processor according to (15-1), in which the deriving unit derives the cognitive capacity on a basis of a task difference in the fluctuation and regression data on the task difference in the fluctuation.

(19)

The information processor according to (15-1), in which the deriving unit derives the cognitive capacity on a basis of the fluctuation and regression data on the fluctuation.

(20)

The information processor according to (15-1), further including a determination unit that determines a task for the user on a basis of the cognitive capacity.

(21)

The information processor according to (20), in which the determination unit changes the task for the user on a basis of the cognitive capacity.

(22)

The information processor according to any one of (15-1), (15-2), (16), (17), (18), (19), (20), and (21), further including an acquisition unit that acquires the biological signal.

(23)

The information processor according to (22), further including a detection unit that detects the biological signal of the user and outputs to the acquisition unit.

(24)

The information processor according to any one of (15-1), (15-2), (16), (17), (18), (19), (20), (21), (22), and (23), further including a presentation unit that presents the request.

(25)

The information processor according to (24), in which the determination unit determines a subsequent request on a basis of a fluctuation in the biological signal.

(26)

The information processor according to (20), in which the determination unit determines a difficulty level of the task on a basis of a fluctuation in the biological signal.

(27)

The information processor according to any one of (15-1), (15-2), (16), (17), (18), (19), (20), (21), (22), (23), (24), (25), and (26), in which the biological signal corresponds to a brain wave, a pulse wave, an electrocardiogram, a blood flow, or emotional sweating of the user.

(28)

The information processor according to any one of (15-1), (15-2), (16), (17), (18), (19), (20), (21), (22), (23), (24), (25), (26), and (27), in which the request corresponds to at least one of game data, item data in healthcare, question data in learning, item data in training for a sports game, or item data in training for human resource development.

(29-1)

An information processor including:

a characteristic value generation unit that generates a characteristic value of a waveform to be observed for each of pieces of observation data, on a basis of a plurality of pieces of partial observation data in an observation period shorter than a predetermined observation period of each of the pieces of observation data obtained by biological observation of a user in the predetermined period, the plurality of pieces of partial observation data being included in each of the pieces of observation data;

an evaluation value generation unit that generates an evaluation value for a difference between the pieces of observation data regarding the waveform to be observed on a basis of the characteristic value generated by the characteristic value generation unit for each of the pieces of observation data; and

a determination unit that determines a task for the user on a basis of a cognitive capacity of the user obtained on a basis of the evaluation value generated by the evaluation value generation unit.

(29-2)

An information processor including:

a characteristic value generation unit that generates a characteristic value of a waveform to be observed for each of pieces of observation data, on a basis of a plurality of pieces of partial observation data in an observation period shorter than a predetermined observation period of each of the pieces of observation data obtained by biological observation of a user in the predetermined period, the plurality of pieces of partial observation data being included in each of the pieces of observation data;

an evaluation value generation unit that generates an evaluation value for a difference between the pieces of observation data regarding the waveform to be observed on a basis of the characteristic value generated by the characteristic value generation unit for each of the pieces of observation data; and

a determination unit that determines a task for the user on a basis of the evaluation value generated by the evaluation value generation unit.

(30)

The information processor according to any one of (29-1) and (29-2), further including a deriving unit that derives the cognitive capacity of the user on a basis of the evaluation value.

(31)

The information processor according to (24), in which the deriving unit derives the cognitive capacity on a basis of a task difference in the evaluation value and regression data on the task difference in the evaluation value.

(32)

The information processor according to any one of (29-1), (29-2), (30), and (31), further including a presentation unit that presents a request.

(33)

The information processor according to (32), in which the determination unit determines a subsequent request on a basis of the evaluation value.

(34)

The information processor according to any one of (29-1), (29-2), (30), (31), (32), (33), and (34), in which the determination unit determines a difficulty level of the task on a basis of the evaluation value.

(35)

The information processor according to (34), in which the determination unit changes the task for the user on a basis of fluctuation in the biological signal.

(36)

The information processor according to any one of (29-1), (29-2), (30), (31), (32), (33),

(34), and (35), in which each of the pieces of observation data corresponds to a brain wave, a pulse wave, an electrocardiogram, a blood flow, or emotional sweating of the user.
(37)

The information processor according to any one of (29-1), (29-2), (30), (31), (32), (33),

(34), (35), and (36), further including a detection unit that detects each of the pieces of observation data.
(38)

The information processor according to any one of (29-1), (29-2), (30), (31), (32), (33),

(34), (35), (36), and (37), in which the request corresponds to at least one of game data, item data in healthcare, question data in learning, item data in training for a sports game, or item data in training for human resource development.
(39-1)

An information processing program that causes a computer to determine a task for a user on a basis of a cognitive capacity of the user obtained on a basis of dispersion of reaction times of the user for a plurality of requests.

(39-2)

An information processing program that causes a computer to determine a task for a user on a basis of dispersion of reaction times of the user for a plurality of requests.

(39-3)

An information processing program that causes a computer to change a task for a user on a basis of dispersion of reaction times of the user for a plurality of requests.

(39-4)

An information processing program that causes a computer to determine a task for a user on a basis of a cognitive capacity of the user obtained on a basis of a biological signal of the user for a request.

(39-5)

An information processing program that causes a computer to derive a cognitive capacity of a user on a basis of a biological signal of the user for a request.

(39-6)

An information processing program that causes a computer to determine a task for a user on a basis of a biological signal of the user for a request.

(39-7)

An information processing program that causes a computer to:

generate a characteristic value of a waveform to be observed for each of pieces of observation data, on a basis of a plurality of pieces of partial observation data in an observation period shorter than a predetermined observation period of each of the pieces of observation data obtained by biological observation of a user in the predetermined period, the plurality of pieces of partial observation data being included in each of the pieces of observation data;

generate an evaluation value for a difference between the pieces of observation data regarding the waveform to be observed on a basis of the generated characteristic value for each of the pieces of observation data; and

determine a task for the user on a basis of a cognitive capacity of the user obtained on a basis of the generated evaluation value.

(39-8)

An information processing program that causes a computer to:

generate a characteristic value of a waveform to be observed for each of pieces of observation data, on a basis of a plurality of pieces of partial observation data in an observation period shorter than a predetermined observation period of each of the pieces of observation data obtained by biological observation of a user in the predetermined period, the plurality of pieces of partial observation data being included in each of the pieces of observation data;

generate an evaluation value for a difference between the pieces of observation data regarding the waveform to be observed on a basis of the generated characteristic value for each of the pieces of observation data; and

determine a task for the user on a basis of the generated evaluation value.

In the information processor according to a first aspect of the present disclosure, a task for a user is determined on the basis of dispersion of reaction times of the user corresponding to a plurality of requests. Here, the present discloser has experimentally obtained knowledge that the dispersion of the reaction times varies depending on tasks. It is therefore possible to determine a task for the user on the basis of the dispersion of the reaction times. Thus, it is possible to detect the cognitive capacity regardless of whether or not there is correct reaction time.

In the information processor according to a second aspect of the present disclosure, a task for a user is determined on the basis of a fluctuation in a biological signal in a specific frequency band of the user for a request. Here, the present discloser has experimentally obtained knowledge that the fluctuation in the biological signal in the specific frequency band of the user varies depending on tasks. It is therefore possible to determine a task for the user on the basis of the fluctuation in the biological signal in the specific frequency band of the user. Thus, it is possible to detect the cognitive capacity regardless of whether or not there is reaction time.

In the information processor according to a third aspect of the present disclosure, a characteristic value for each of pieces of observation data is derived from each of the pieces of observation data obtained by biological observation of a user in a predetermined period, and an evaluation value for a difference between the pieces of observation data regarding an waveform to be observed is generated on the basis of the derived characteristic value for each of the pieces of observation data. Then, a task for the user is determined on the basis of the generated evaluation value. Here, the present discloser has experimentally obtained knowledge that the above-described evaluation value varies depending on tasks. It is therefore possible to determine a task for the user on the basis of the above-described evaluation value. Thus, it is possible to detect the cognitive capacity regardless of whether or not there is reaction time.

In the information processing program according to a fourth aspect of the present disclosure, a task for a user is determined on the basis of dispersion of reaction times of the user for a plurality of requests. Here, the present discloser has experimentally obtained knowledge that the dispersion of the reaction times varies depending on tasks. It is therefore possible to determine a task for the user on the basis of the dispersion of the reaction times. Thus, it is possible to detect the cognitive capacity regardless of whether or not there is correct reaction time.

In the information processor program according to a fifth aspect of the present disclosure, a task for a user is determined on the basis of a biological signal of the user for a request. Here, the present discloser has experimentally obtained knowledge that the biological signal of the user varies depending on tasks. It is therefore possible to determine a task for the user on the basis of the biological signal of the user. Thus, it is possible to detect the cognitive capacity regardless of whether or not there is reaction time.

In the information processing program according to a sixth aspect of the present disclosure, a characteristic value for each of pieces of observation data is derived from each of the pieces of observation data obtained by biological observation of a user in a predetermined period, and an evaluation value for a difference between the pieces of observation data regarding an waveform to be observed is generated on the basis of the derived characteristic value for each of the pieces of observation data. Then, a task for the user is determined on the basis of the generated evaluation value. Here, the present discloser has experimentally obtained knowledge that the above-described evaluation value varies depending on tasks. It is therefore possible to derive a cognitive capacity of the user on the basis of the above-described evaluation value, and to determine a task for the user on the basis of the derived cognitive capacity. Thus, it is possible to detect the cognitive capacity regardless of whether or not there is reaction time.

In the information processor according to a seventh aspect of the present disclosure, a task for a user is changed on the basis of dispersion of reaction times of the user corresponding to a plurality of requests. Here, the present discloser has experimentally obtained knowledge that the dispersion of the reaction times varies depending on tasks. It is therefore possible to change a task for the user on the basis of the dispersion of the reaction times. Thus, it is possible to detect the cognitive capacity regardless of whether or not there is correct reaction time.

In the information processor according to an eighth aspect of the present disclosure, a task for a user is changed on the basis of a fluctuation in a biological signal of the user for a request. Here, the present discloser has experimentally obtained knowledge that the biological signal of the user varies depending on tasks. It is therefore possible to change a task for the user on the basis of the fluctuation in the biological signal of the user. Thus, it is possible to detect the cognitive capacity regardless of whether or not there is correct reaction time.

This application claims the benefits of Japanese Priority Patent Application JP2020-072585 filed with the Japan Patent Office on Apr. 14, 2020, and Japanese Priority Patent Application JP2020-203058 filed with the Japan Patent Office on Dec. 7, 2020, the entire contents of which are incorporated herein by reference.

It should be understood by those skilled in the art that various modifications, combinations, sub-combinations, and alterations may occur depending on design requirements and other factors insofar as they are within the scope of the appended claims or the equivalents thereof.

What is claimed is:

1. An information processor comprising a deriving unit that derives a cognitive capacity (cognitive resource) of a user on a basis of dispersion of reaction times of the user for a plurality of requests.

2. The information processor according to claim 1, wherein the deriving unit derives the cognitive capacity on a basis of a task difference in the dispersion of the reaction times and regression data on the task difference in the dispersion of the reaction times.

3. The information processor according to claim 1, wherein the deriving unit derives the cognitive capacity on a basis of the dispersion of the reaction times and regression data on the dispersion of the reaction times.

4. The information processor according to claim 1, further comprising a determination unit that determines a task for the user on a basis of the cognitive capacity.

5. The information processor according to claim 4, wherein the determination unit changes the task for the user on a basis of the cognitive capacity.

6. The information processor according to claim 1, further comprising an acquisition unit that acquires the reaction times.

7. The information processor according to claim 6, wherein the acquisition unit derives the reaction times on a basis of answer input timings or action logs of the user for the plurality of requests.

8. The information processor according to claim 4, further comprising a presentation unit that presents the plurality of requests.

9. The information processor according to claim 8, wherein the determination unit determines a plurality of subsequent requests on a basis of the dispersion of the reaction times.

10. The information processor according to claim 4, wherein the determination unit determines a difficulty level of the task on a basis of the dispersion of the reaction times.

11. The information processor according to claim 10, wherein the determination unit changes the difficulty level of the task on a basis of the dispersion of the reaction times.

12. The information processor according to claim 1, wherein the requests correspond to at least one of game data, item data in healthcare, question data in learning, item data in training for a sports game, or item data in training for human resource development.

13. An information processor comprising a deriving unit that derives a cognitive capacity of a user on a basis of a biological signal of the user for a request.

14. The information processor according to claim 13, wherein the biological signal comprises time series data.

15. The information processor according to claim 14, wherein the deriving unit derives the cognitive capacity of the user on a basis of a fluctuation in a component in a specific frequency band included in the biological signal.

16. The information processor according to claim 13, wherein the deriving unit derives the cognitive capacity on a basis of a task difference in the fluctuation and regression data on the task difference in the fluctuation.

17. The information processor according to claim 13, wherein the deriving unit derives the cognitive capacity on a basis of the fluctuation and regression data on the fluctuation.

18. The information processor according to claim 13, further comprising a determination unit that determines a task for the user on a basis of the cognitive capacity.

19. The information processor according to claim 18, wherein the determination unit changes the task for the user on a basis of the cognitive capacity.

20. The information processor according to claim 13, further comprising an acquisition unit that acquires the biological signal.

21. The information processor according to claim 20, further comprising a detection unit that detects the biological signal of the user and outputs to the acquisition unit.

22. The information processor according to claim 13, further comprising a presentation unit that presents the request.

23. The information processor according to claim 22, wherein the determination unit determines a subsequent request on a basis of a fluctuation in the biological signal.

24. The information processor according to claim 13, wherein the determination unit determines a difficulty level of the task on a basis of a fluctuation in the biological signal.

25. The information processor according to claim 24, wherein the determination unit changes the task for the user on a basis of the fluctuation in the biological signal.

26. The information processor according to claim 13, wherein the biological signal corresponds to a brain wave, a pulse wave, an electrocardiogram, a blood flow, or emotional sweating of the user.

27. The information processor according to claim 13, wherein the request corresponds to at least one of game data, item data in healthcare, question data in learning, item data in training for a sports game, or item data in training for human resource development.

28. An information processor, comprising:
a characteristic value generation unit that generates a characteristic value of a waveform to be observed for each of pieces of observation data, on a basis of a plurality of pieces of partial observation data in an observation period shorter than a predetermined observation period of each of the pieces of observation data obtained by biological observation of a user in the predetermined period, the plurality of pieces of partial observation data being included in each of the pieces of observation data;
an evaluation value generation unit that generates an evaluation value for a difference between the pieces of observation data regarding the waveform to be observed on a basis of the characteristic value generated by the characteristic value generation unit for each of the pieces of observation data; and
a deriving unit that derives a cognitive capacity of the user on a basis of the evaluation value generated by the evaluation value generation unit.

29. The information processor according to claim 28, wherein the deriving unit derives the cognitive capacity on a basis of a task difference in the evaluation value and regression data on the task difference in the evaluation value.

30. The information processor according to claim 28, wherein the deriving unit derives the cognitive capacity on a basis of the evaluation value and regression data on the evaluation value.

31. The information processor according to claim 28, further comprising a determination unit that determines a task for the user on a basis of the cognitive capacity.

32. The information processor according to claim 31, wherein the determination unit changes the task for the user on a basis of the cognitive capacity.

33. The information processor according to claim 28, further comprising a presentation unit that presents a request.

34. The information processor according to claim 33, wherein the determination unit determines a subsequent request on a basis of the evaluation value.

35. The information processor according to claim 28, wherein the determination unit determines a difficulty level of the task on a basis of the evaluation value.

36. The information processor according to claim 28, wherein each of the pieces of observation data corresponds to a brain wave of the user.

37. The information processor according to claim 28, further comprising a detection unit that detects each of the pieces of observation data.

38. The information processor according to claim 28, wherein the request corresponds to at least one of game data, item data in healthcare, question data in learning, item data in training for a sports game, or item data in training for human resource development.

39. An information processing program that causes a computer to derive a cognitive capacity of a user on a basis of dispersion of reaction times of the user for a plurality of requests.

40. An information processing program that causes a computer to derive a cognitive capacity of a user on a basis of a biological signal of the user for a request.

41. An information processing program that causes a computer to:
generate a characteristic value of a waveform to be observed for each of pieces of observation data, on a basis of a plurality of pieces of partial observation data in an observation period shorter than a predetermined observation period of each of the pieces of observation data obtained by biological observation of a user in the predetermined period, the plurality of pieces of partial observation data being included in each of the pieces of observation data;
generate an evaluation value for a difference between the pieces of observation data regarding the waveform to be observed on a basis of the generated characteristic value for each of the pieces of observation data; and
derive a cognitive capacity of the user on a basis of the generated evaluation value.

42. An information processor comprising a changing unit that changes a task for a user on a basis of dispersion of reaction times of the user for a plurality of requests.

43. The information processor according to claim 42, further comprising an acquisition unit that acquires the reaction times.

44. The information processor according to claim 43, wherein the acquisition unfit acquires the reaction times on a basis of information from a sensor.

45. The information processor according to claim 44, wherein the acquisition unit derives the reaction times on a basis of answer input timings or action logs of the user for the plurality of requests.

46. The information processor according to claim 42, wherein the requests correspond to at least one of game data, item data in healthcare, question data in learning, item data in training for a sports game, or item data in training for human resource development.

47. An information processor comprising a changing unit that changes a task for a user on a basis of a fluctuation in a biological signal of the user for a request.

48. The information processor according to claim 47, wherein the fluctuation in the biological signal of the user comprises a fluctuation in a component in a specific frequency band included in the biological signal of the user.

49. The information processor according to claim 47, wherein the biological signal corresponds to a brain wave, a pulse wave, an electrocardiogram, a blood flow, or emotional sweating of the user.

50. The information processor according to claim 47, wherein the request corresponds to at least one of game data, item data in healthcare, question data in learning, item data in training for a sports game, or item data in training for human resource development.

51. The information processor according to claim 47, wherein a difficulty level of the task for the user is changed on a basis of the fluctuation in the biological signal of the user for the request.
